(19) United States
(12) Patent Application Publication
(10) Pub. No.: US 2022/0081722 A1
(43) Pub. Date: Mar. 17, 2022

(54) Method of Diagnosing Gastric Cancers Using MicroRNAs

(71) Applicants: AGENCY FOR SCIENCE, TECHNOLOGY AND RESEARCH, Singapore (SG); MIRXES LAB PTE LTD, Singapore (SG)

(72) Inventors: Heng Phon Too, Singapore (SG); Ka Yan Chung, Singapore (SG); Jia Min Quek Yang Zou, Singapore (SG); Rui Yang Zou, Singapore (SG)

(21) Appl. No.: 17/415,881

(22) PCT Filed: Dec. 17, 2019
(86) PCT No.: PCT/SG2019/050621

§ 371 Date: Jun. 18, 2021

(30) Foreign Application Priority Data
Dec. 19, 2018 (SG) 10201811421Q

(51) Int. Cl. C12Q 1/6886; C12N 15/113; A61P 35/00

(52) U.S. Cl. CPC C12Q 1/6886; C12N 15/113; C12Q 2600/178; C12N 2310/141; C12Q 2600/158; A61P 35/00

(57) ABSTRACT

We describe a method of diagnosing a gastric cancer. The method may include detecting the expression level of an miRNA in a sample of or from an individual. The expression level of the miRNA may be detected in an extracellular vesicle (EV) from the sample. The miRNA may be selected from the group consisting of: hsa-miR-484, hsa-miR-186-5p, hsa-miR-142-5p, hsa-miR-320d, hsa-miR-320a, hsa-miR-320b and hsa-miR-17-5p. The method may be such that an altered expression level of the miRNA as compared to the expression level of the miRNA in an EV in or of an individual known not to be suffering from gastric cancer indicates that the individual is suffering, or is likely to be suffering, from gastric cancer.

FIELD

This invention relates to the fields of medicine, cell biology, molecular biology and genetics.

### BACKGROUND

MicroRNAs (miRNAs) are small non-coding RNAs (˜19-22 nucleotides) that regulate protein expression and exert physiological significance in several key cellular processes, such as cell differentiation, proliferation and apoptosis. Circulating miRNAs, which can be readily detected in biofluids such as serum, plasma or whole blood, are promising liquid biopsy biomarkers for non-invasive detection of various diseases, including cancer. In addition, aberrations affecting miRNAs have been shown to significantly affect cancer genesis and progression.

MiRNAs have good potential to be used as circulating biomarkers of diseases due to their stability in serum/plasma, substantial attention and tremendous efforts have been dedicated to identify miRNA biomarkers for early detection, prognosis or therapeutic purposes.

However, changes in the miRNA expression level might be subtle during the onset of disease, thus making diagnosis challenging. An ideal liquid biopsy biomarker should have a high signal-to-noise ratio between cancer and control samples, which can be readily detectable in clinical settings. The current challenge is therefore to measure the small differences in the miRNA expression levels in disease and healthy individuals. Detection of subtle, but meaningful differences in circulating miRNA quantities between diseased and healthy samples remains a key challenge in clinical settings since biomarker signal-to-noise ratios are often low.

There is substantial variability in the miRNA signatures identified in different studies. One such reason for variation is the fact that sample handling can affect the degree of haemolysis in blood samples, leading to release of miRNAs from lysed RBCs and therefore changing the miRNA profile.

Extracellular vesicles (EVs) play an important role in cellular communication and promote tumour development. miRNA expression in EVs is frequently dysregulated and cancer cells may actively secrete miRNA-containing EVs into the cancer microenvironment. Isolation of miRNAs from EVs therefore may potentially reduce the potential contaminating miRNAs present in the biofluids and is therefore useful for early diagnosis of cancer or other diseases. Isolation of EVs can however be laborious and time-consuming which limits its application in clinical settings. To date, there has been no systematic and comprehensive evaluation of EV-miRNA isolation methods and there is no standardized protocol for EV-miRNA isolation.

### SUMMARY

While miRNA has been proposed for diagnosis of cancer, including gastric cancer, there has thus far not been very good correlation in the miRNA signatures identified in the different studies for the same disease.

For example, Leidner et al (2013) and Tiberio et al (2015) provide examples of the issues faced in using miRNAs as diagnostic markers.

Leidner RS, Li L, Thompson C L. Dampening enthusiasm for circulating microRNA in breast cancer. PLoS One. 2013;8(3):e57841. doi: 10.1371/journal.pone.0057841

Tiberio P, Callari M, Angeloni V, Daidone M G, Appierto V. Challenges in using circulating miRNAs as cancer biomarkers. Biomed Res Int 2015;2015:731479. doi: 10.1155/2015/731479

In contrast, the claimed invention allows for improvements that could enhance the signal-to-noise ratio, hence more reliably identifying miRNAs differentially expressed in gastric cancer and therefore leading to improved diagnostic performance for the claimed assay over those in the art.

Furthermore, many of the symptoms associated with gastric cancer are not gastric cancer specific. It is therefore not straightforward to identify gastric cancer until a late stage where the severity of the symptoms would lead physicians to perform the relevant imaging or endoscopic tests.

Survival rates of patients treated early are far better than those found with advanced cancer. Therefore, there is value in a reliable test that may be used for early diagnosis of gastric cancer.

As shown in Pasechnikov et al (2014) and Kim et al, current screening methodologies usually use either invasive (biopsy or endoscopy) or imaging-based methods (which often exposes to the patients to radioactivity and/or is costly).

Pasechnikov V, Chukov S, Fedorov E, Kikuste I, Leja M. Gastric cancer: prevention, screening and early diagnosis. World J Gastroenterol. 2014;20(38): 13842-13862. doi: 10.3748/wjg.v20.i38.13842

Kim G H, Liang P S, Bang S J, Hwang J H. Screening and surveillance for gastric cancer in the United States: Is it needed? Gastrointest Endosc. 2016 Jul;84(1): 18-28. doi: 10.1016/j.gie.2016.02.028. Epub 2016 Mar 3.

The currently available non-invasive tests like pepsinogen, H. pylori serology, etc do not provide the requisite specificity and sensitivity.

We therefore provide for a non-invasive methodology that is an improvement over those currently available, and which would be useful for diagnosing gastric cancer in the population at large.

In some embodiments, a positive diagnosis using this test may lead to further confirmatory tests using the more invasive gold-standard tests such as endoscopy or biopsy. This would also reduce the number of patients needing to undergo such invasive and costly tests in the first place.

According to a 1^{st} aspect of the present invention, we provide a method of diagnosing a gastric cancer. The method may include detecting the expression level of an miRNA in a sample from or of an individual.

The miRNA may be selected from the group consisting of: hsa-miR-484, hsa-miR-186-5p, hsa-miR-142-5p, hsa-miR-320d, hsa-miR-320a, hsa-miR-320b and hsa-miR-17-5p. The method may be such that an altered expression level of the miRNA as compared to the expression level of the miRNA in a sample from or of an individual known not to be suffering from gastric cancer indicates that the individual is suffering, or is likely to be suffering, from gastric cancer.

The expression level of the miRNA may be detected in an extracellular vesicle (EV) in a sample from or of the individual.

The miRNA may comprise hsa-miR-484. hsa-miR-484 may comprise a polynucleotide sequence having miRBase Accession Number MIMAT0002174. hsa-miR-484 may also comprise a variant, homologue, derivative or fragment thereof. The variant, homologue, derivative or fragment thereof may comprise a sequence having 75%, 80%, 85%, 90%, 95%, 96%, 97%, 98% or 99% sequence identity to hsa-miR-484. The variant, homologue, derivative or fragment thereof may comprise hsa-miR-484 activity. The altered expression level may comprise an increased expression level of hsa-miR-484.

The miRNA may comprise hsa-miR-186-5p. hsa-miR-186-5p may comprise a polynucleotide sequence having miRBase Accession Number MIMAT0000456. hsa-miR-186-5p may also comprise a variant, homologue, derivative or fragment thereof. The variant, homologue, derivative or fragment thereof may comprise a sequence having 75%, 80%, 85%, 90%, 95%, 96%, 97%, 98% or 99% sequence identity to hsa-miR-186-5p. The variant, homologue, derivative or fragment thereof may comprise hsa-miR-186-5p activity. The altered expression level may comprise an increased expression level of hsa-miR-186-5p.

The miRNA may comprise hsa-miR-142-5p. hsa-miR-142-5p may comprise a polynucleotide sequence having miRBase Accession Number MIMAT0000433. hsa-miR-142-5p may also comprise a variant, homologue, derivative or fragment thereof. The variant, homologue, derivative or fragment thereof may comprise a sequence having 75%, 80%, 85%, 90%, 95%, 96%, 97%, 98% or 99% sequence identity to hsa-miR-142-5p. The variant, homologue, derivative or fragment thereof may comprise hsa-miR-142-5p activity. The altered expression level may comprise a decreased expression level of hsa-miR-142-5p.

The miRNA may comprise hsa-miR-320d. hsa-miR-320d may comprise a polynucleotide sequence having miRBase Accession Number MIMAT0006764. hsa-miR-320d may also comprise a variant, homologue, derivative or fragment thereof. The variant, homologue, derivative or fragment thereof may comprise a sequence having 75%, 80%, 85%, 90%, 95%, 96%, 97%, 98% or 99% sequence identity to hsa-miR-320d. The variant, homologue, derivative or fragment thereof may comprise hsa-miR-320d activity. The altered expression level may comprise an increased expression level of hsa-miR-320d.

The miRNA may comprise hsa-miR-320a. hsa-miR-320a may comprise a polynucleotide sequence having miRBase Accession Number M10000542. hsa-miR-320a may also comprise a variant, homologue, derivative or fragment thereof. The variant, homologue, derivative or fragment thereof may comprise a sequence having 75%, 80%, 85%, 90%, 95%, 96%, 97%, 98% or 99% sequence identity to hsa-miR-320a. The variant, homologue, derivative or fragment thereof may comprise hsa-miR-320a activity. The altered expression level may comprise an increased expression level of hsa-miR-320a.

The miRNA may comprise hsa-miR-320b. hsa-miR-320b may comprise a polynucleotide sequence having miRBase Accession Number MIMAT0005792. hsa-miR-320b may also comprise a variant, homologue, derivative or fragment thereof. The variant, homologue, derivative or fragment thereof may comprise a sequence having 75%, 80%, 85%, 90%, 95%, 96%, 97%, 98% or 99% sequence identity to hsa-miR-320b. The variant, homologue, derivative or fragment thereof may comprise hsa-miR-320b activity. The altered expression level may comprise an increased expression level of hsa-miR-320b.

The miRNA may comprise hsa-miR-17-5p. hsa-miR-17-5p may comprise a polynucleotide sequence having miRBase Accession Number MIMAT0000070. hsa-miR-17-5p may also comprise a variant, homologue, derivative or fragment thereof. The variant, homologue, derivative or fragment thereof may comprise a sequence having 75%, 80%, 85%, 90%, 95%, 96%, 97%, 98% or 99% sequence identity to hsa-miR-17-5p. The variant, homologue, derivative or fragment thereof may comprise hsa-miR-17-5p activity. The altered expression level may comprise a decreased expression level of hsa-miR-17-5p.

The method may comprise detecting the expression level in a sample, such as in an extracellular vesicle (EV) in or of the sample, of two or more such miRNAs. The method may comprise detecting the expression level in a sample, such as in an extracellular vesicle (EV) in or of the sample, of three such miRNAs. The method may comprise detecting the expression level in a sample, such as in an extracellular vesicle (EV) in or of the sample, of four such miRNAs. The method may comprise detecting the expression level in a sample, such as in an extracellular vesicle (EV) in or of the sample, of five such miRNAs. The method may comprise detecting the expression level in a sample, such as in an extracellular vesicle (EV) in or of the sample, of six such miRNAs. The method may comprise detecting the expression level in a sample, such as in an extracellular vesicle (EV) in or of the sample, of seven such miRNAs.

The method may further comprise detecting the expression level in a sample, such as in an extracellular vesicle (EV) in or of the sample, of a further miRNA in combination with any one or more of miRNAs selected from the group consisting: hsa-miR-484, hsa-miR-186-5p, hsa-miR-142-5p, hsa-miR-320d, hsa-miR-320a, hsa-miR-320b and hsa-miR-17-5p. The further miRNA may comprise hsa-miR-423-5p (miRBase Accession Number MIMAT0004748). The further miRNA may comprise a variant, homologue, derivative or fragment of hsa-miR-423-5p such as a sequence having at least 75%, 80%, 85%, 90%, 95%, 96%, 97%, 98% or 99% sequence identity to hsa-miR-423-5p. Such a variant, homologue, derivative or fragment of hsa-miR-423-5p may comprise hsa-miR-423-5p activity.

Gastric cancer may be indicated where an expression level of hsa-miR-484 of 0.39 or more is detected. Gastric cancer may be indicated where an expression level of hsa-miR-186-5p of 0.15 or more, measured as log₂(expression level of individual/expression level of control), in which control designates the expression level of that miRNA in a sample, such as in an extracellular vesicle (EV) in or of the sample, from or of an individual known not to be suffering from gastric cancer is detected.

Gastric cancer may be indicated where an expression level of hsa-miR-142-5p of −0.35 or less, measured as log₂(expression level of individual/expression level of control), in which control designates the expression level of that miRNA in a sample, such as in an extracellular vesicle (EV) in or of the sample, from or of an individual known not to be suffering from gastric cancer, is detected.

Gastric cancer may be indicated where an expression level of hsa-miR-320d of 0.48 or more, measured as log₂(expression level of individual/expression level of control), in which control designates the expression level of that miRNA in a sample, such as in an extracellular vesicle (EV) in or of the sample, from or of an individual known not to be suffering from gastric cancer, is detected.

Gastric cancer may be indicated where an expression level of hsa-miR-320a of 0.49 or more, measured as log₂(expression level of individual/expression level of control), in which control designates the expression level of that miRNA in a sample, such as in an extracellular vesicle (EV) in or of the sample, from or of an individual known not to be suffering from gastric cancer, is detected.

Gastric cancer may be indicated where an expression level of hsa-miR-320b of 0.4 or more, measured as log₂(expression level of individual/expression level of control), in which control designates the expression level of that miRNA in a sample, such as in an extracellular vesicle (EV) in or of the sample, from or of an individual known not to be suffering from gastric cancer, is detected.

Gastric cancer may be indicated where an expression level of hsa-miR-17-5p of -0.37 or less, measured as log₂(expression level of individual/expression level of control), in which control designates the expression level of that miRNA in a sample, such as in an extracellular vesicle (EV) in or of the sample, from or of an individual known not to be suffering from gastric cancer, is detected.

Gastric cancer may be indicated where an expression level of hsa-miR-423-5p of 0.54 or more, measured as log₂(expression level of individual/expression level of control), in which control designates the expression level of that miRNA in a sample, such as in an extracellular vesicle (EV) in or of the sample, from or of an individual known not to be suffering from gastric cancer, is detected.

The sample may comprise a bodily fluid sample. The sample may comprise a nasopharyngeal secretion, urine, serum, lymph, saliva, anal and vaginal secretions, perspiration or semen of the individual.

The extracellular vesicle (EV) in or of the individual may be from a sample in or of the individual. The extracellular vesicle (EV) may be isolated from the sample using polymer based precipitation.

Expression of miRNA may be detected by any means. For example, the miRNA detection may comprise use of a polymerase chain reaction, such as real-time polymerase chain reaction (RT-PCR), multiplex polymerase chain reaction (multiplex PCR). The miRNA detection may be by means of Northern Blot, RNAse protection, microarray hybridisation or RNA sequencing.

There is provided, according to a 2^{nd} aspect of the present invention, a combination of two or more nucleic acids specified above or probes capable of binding specifically thereto. This may comprise a combination of nucleic acids immobilised on a substrate. The combination may be in the form of a microarray or as a multiplex polymerase chain reaction (PCR) kit.

The combination may comprise probes capable of binding specifically thereto to each of hsa-miR-484 (miRBase Accession Number MIMAT0002174), hsa-miR-186-5p (miRbase Accession Number MIMAT0000456), hsa-miR-142-5p (miRBase Accession Number MIMAT0000433), hsa-miR-320d (miRBase Accession Number MIMAT0006764), hsa-miR-320a (miRBase Accession Number MI0000542), hsa-miR-320b (miRBase Accession Number MIMAT0005792), hsa-miR-17-5p (miRBase Accession Number MIMAT0000070) and hsa-miR-423-5p (miRBase Accession Number MIMAT0004748).

We provide, according to a 3^{rd} aspect of the present invention, an miRNA selected from the group consisting of hsa-miR-484, hsa-miR-186-5p, hsa-miR-142-5p, hsa-miR-320d, hsa-miR-320a, hsa-miR-320b and hsa-miR-17-5p or a variant, homologue, derivative or fragment thereof such as a sequence having at least 75%, 80%, 85%, 90%, 95%, 96%, 97%, 98% or 99% sequence identity thereto for use in a method of detecting or determining the severity of gastric cancer.

As a 4^{th} aspect of the present invention, there is provided a pharmaceutical composition comprising two or more miRNAs selected from the group consisting of: hsa-miR-484, hsa-miR-186-5p, hsa-miR-142-5p, hsa-miR-320d, hsa-miR-320a, hsa-miR-320b, hsa-miR-17-5p, and hsa-miR-423-5p, or a variant, homologue, derivative or fragment thereof such as a sequence having at least 75%, 80%, 85%, 90%, 95%, 96%, 97%, 98% or 99% sequence identity thereto together with a pharmaceutically acceptable excipient, carrier or diluent.

We provide, according to a 5^{th} aspect of the present invention, a diagnostic kit for gastric cancer, the kit comprising sequences capable of binding to two or more miRNAs selected from the group consisting of: hsa-miR-484, hsa-miR-186-5p, hsa-miR-142-5p, hsa-miR-320d, hsa-miR-320a, hsa-miR-320b, hsa-miR-17-5p and hsa-miR-423-5p, or a variant, homologue, derivative or fragment thereof such as a sequence having at least 75%, 80%, 85%, 90%, 95%, 96%, 97%, 98% or 99% sequence identity thereto together with instructions for use.

The present invention, in a 6^{th} aspect, provides a method of treatment of a gastric cancer in an individual, the method comprising performing a method as set out above and, where the individual is determined to be suffering from, or likely to suffer from, gastric cancer, administering to the individual a treatment for gastric cancer.

In a 7^{th} aspect of the present invention, there is provided a method of treating gastric cancer in an individual, the method comprising: (a) receiving results of an assay that measures the expression level of an miRNA selected from the group consisting of: hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, or a variant, homologue, derivative or fragment thereof such as a sequence having at least 75%, 80%, 85%, 90%, 95%, 96%, 97%, 98% or 99% sequence identity thereto in a sample of or from an individual, in which the results show the expression level of the miRNA in the sample; (b) if the expression of the miRNA in the sample is modulated compared to a reference expression level of an miRNA, the reference expression level being the expression level of the miRNA in a sample of an individual known not to be suffering from gastric cancer, thereby providing or predicting an indication of gastric cancer in the individual, administering a treatment for gastric cancer. The expression level of the or each miRNA may be measured in an extracellular vesicle (EV) of the or each sample.

According to an 8^{th} aspect of the present invention, we provide a method for treating gastric cancer in an individual, comprising: (a) obtaining the results of an analysis of the expression level of an miRNA selected from the group consisting of: hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, or a variant, homologue, derivative or fragment thereof such as a sequence having at least 75%, 80%, 85%, 90%, 95%, 96%, 97%, 98% or 99% sequence identity thereto in a sample of or from an individual; and (b) administering a treatment for gastric cancer to the individual if the expression level of the miRNA is modulated compared to a reference expression level, the reference expression level being the expression level of the miRNA in a sample of or from an individual known not to be suffering from gastric cancer. The expression level of the or each miRNA may be measured in an extracellular vesicle (EV) of the or each sample.

The practice of this invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O′D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

### BRIEF DESCRIPTION OF THE FIGURES

FIG. 1A and FIG. 1B are diagrams showing miRNA profiles from EV fractions.

FIG. 1A. EVs were isolated using UC (black), column affinity (white), peptide affinity (light grey) and immunobead affinity (dark grey) method from 200 μl pooled human serum. RNA were then extracted from these EV fractions as well as from 200 μl serum (total) using QIAzol lysis reagent and 11 miRNAs were quantified by RT-qPCR. Percentage EV miRNA recovery were calculated using 2^{-(CTtotal-CTEV}) . Each experimental condition was carried out thrice and data were presented as mean±SEM.

FIG. 1B. Western blot analysis of EVs isolated using UC, column affinity and peptide affinity. The expression of common EV markers Flotillin, TSG101 and CD9 were presented.

FIG. 2A and FIG. 2B are diagrams showing miRNA profiles from EV fractions.

FIG. 2A. EVs were isolated using UC (black), Invt (white), SBI (light grey), Exi (dark grey) and Han (brown) method from 200 μl pooled human serum. RNA were then extracted from these EV fractions as well as from 200 μl serum (total) using QIAzol lysis reagent and 11 miRNAs were quantified by RT-qPCR. Percentage EV miRNA recovery were calculated using 2^{-(CTtotal-CTEV}). Each experimental condition was carried out thrice and data were presented as mean±SEM.

FIG. 2B. Western blot analysis of EVs isolated using UC and polymer-based precipitation method. The expression of common EV markers Flotillin, TSG101, CD9, CD63 and CD81 were presented.

FIG. 3 is a diagram showing miRNA profiling using four different polymer-based precipitation methods. miRNA fold-change between total and EV-fractions was calculated based on [log₂(copy no_{EV}/copy noₜₒₜₐₗ)] in gastric cancer (Can) and control (Ctrl) group, for all four methods.

FIG. 4 is a diagram showing miRNA profiling using four different polymer-based precipitation methods. Comparison between the p-value of fold-change between cancer and control for each polymer-based precipitation methods with p-value in total fraction. EV associated miRNAs with a p-value <0.05 and a p-value <total fraction were circled.

FIG. 5 is a diagram showing box plots showing the expression levels of the eight miRNAs identified to increase signal-to-noise ratio in cancer and control group.

FIG. 6A, FIG. 6B and FIG. 6C are diagrams showing miRNA profiles from EV fractions.

FIG. 6A and FIG. 6B are diagrams showing that EVs were isolated from different methods using 200 μI pooled human serum. RNA was then extracted from these EV fractions as well as from 200 μI serum using QIAzol lysis reagent and 11 miRNAs were quantified by RT-qPCR. Each box plot represents the percentage of miRNA recovery (from total serum) from all 11 miRNAs measured. Percentage recovery was calculated using 2^{-(Ct}ₜₒₜₐₗ^{-Ct}_{EV}). Each experimental condition was carried out thrice and data were presented as mean±SEM. “+” indicated outlier data point.

FIG. 6C is a diagram showing Western blot analysis of EVs isolated using PBP. The expression of common EV markers Flotillin, TSG101, CD9, CD63 and CD81 were presented.

FIG. 7A, FIG. 7B are diagrams showing miRNA profiling using 4 different PBP reagents.

FIG. 7A is a diagram showing a comparison between the p-value of fold-change between cancer and control for each reagent with p-value in total fraction. EV-associated miRNAs with a p-value <0.05 and a p-value <total fraction was circled. Dashed lines indicated log₁₀(0.05) as a cut-off.

FIG. 7B is a diagram showing the AUC of miRNAs isolated using Invt (white bar) compared to total serum (black bar).

FIG. 8A and FIG. 8B are diagrams showing miRNAs with better diagnostic performance in EV compared to total serum.

FIG. 8A is a diagram showing miRNA fold-change (calculated based on [log2(Copy No_{EV}. Copy Noₜₒₜₐₗ)]) in gastric cancer and control group was presented in total serum (black bar) and Invt (white bar).

FIG. 8B is a diagram showing AUC of miRNAs isolated using Invt (white bar) compared to total serum (black bar).

FIG. 9 is a diagram showing the median AUC values for the use of individual miRNAs (1-miRNA panel) or a combination of a number of miRNAs for the detection of gastric cancer in total serum (Total) or in isolated extracellular vesicles (EV). In the case of the 8-miRNA panel, a single AUC value is provided instead of a median value in view of the fact that only a single combination is possible.

### DETAILED DESCRIPTION

EV-associated miRNAs are of interest because they play an important role in cellular communication process and tumour development. Expression level of in EV-associated miRNAs is frequently dysregulated during cancer development/progression. Isolating EV fractions may therefore enhance cancer diagnostic potential.

Because extracellular vesicles (EVs) are a key source of circulating miRNAs in serum, we hypothesized that isolating EVs will enrich miRNA biomarkers, leading to enhanced diagnostic ability and improved biomarker performance.

We sought to identify a suitable high-throughput method for the rapid isolation of EV-associated miRNA and to test the hypothesis that this fraction can improve the detection of these miRNAs in serum. We further sought to identify EV-associated miRNA that can potentially serve as non-invasive diagnostic tool for the gastric cancer (GC) detection.

In this study, we assessed the performance of EV-miRNAs against serum miRNAs as biomarkers for gastric cancer (GC).

We evaluated 5 different isolation methods and selected polymer-based precipitation (PBP) approach to isolate EV from 15 GC and 15 matched healthy control serum. As a pilot study, a panel of 133 GC-related miRNAs was measured in both the total serum and EV fractions. Of these miRNAs, 11 were significantly different between cancer and control. In a separate validation set using 20 independent pairs of cancer and control serum, 8 out of the 11 candidates were found to enhance the diagnostic potential of miRNA in EV fractions as compared to total serum. Overall, we demonstrated that the enrichment of miRNAs in EVs can significantly enhance the sensitivity of miRNA biomarkers in detecting GC, suggesting the potential use of EV-miRNAs in the diagnosis of GC.

In detail, we first determined that polymer-based precipitation (PBP) gave the highest EV-miRNAs recovery when compared to ultracentrifugation, column affinity, peptide affinity, and immunobead affinity EV purification.

We then used four PBP reagents to isolate EV-miRNAs from 15 GC and 15 healthy controls and profiled 133 GC-related miRNAs from EV fractions and whole serum using RT-qPCR. We selected a PBP reagent which generated the most EV-miRNA biomarkers and used it to validate 11 EV-miRNAs in an independent set of 20 GC and 20 controls.

Eight of these EV-miRNA biomarkers were found to give better GC detection accuracy (AUC>0.8). Use of these 8 miRNAs significantly improves the sensitivity and specificity for GC diagnosis. No other reports have been published on the use of these 8 miRNAs for cancer diagnosis.

Overall, we showed that EV miRNAs can improved GC detection performance compared to serum miRNAs and led to the identification of 8 EV-miRNAs as potential non-invasive biomarkers for GC.

Our method of EV isolation and subsequent miRNA detection is easily adaptable into clinical setting.

### Use of Mirnas in the Diagnosis of Gastric Cancer

We demonstrate, for the first time, that miRNAs such as hsa-miR-484, hsa-miR-186-5p, hsa-miR-142-5p, hsa-miR-320d, hsa-miR-320a, hsa-miR-320b and hsa-miR-17-5p play a role in cancer.

Specifically, we show that hsa-miR-484, hsa-miR-186-5p, hsa-miR-142-5p, hsa-miR-320d, hsa-miR-320a, hsa-miR-320b and hsa-miR-17-5p miRNAs play are differently expressed in gastric cancer cells.

For example, the data disclosed in this application show that expression of hsa-miR-484, has-miR-186-5p, hsa-miR-320d, hsa-miR-320a or hsa-miR-320b is increased in a patient suffering from (or likely to suffer from) gastric cancer, compared to an individual known not to be suffering from gastric cancer.

Furthermore, the data disclosed in this application show that expression of hsa-miR-142-5p and hsa-miR-17-5p is decreased in a patient suffering from (or likely to suffer from) gastric cancer, compared to an individual known not to be suffering from gastric cancer.

Accordingly, hsa-miR-484, hsa-miR-186-5p, hsa-miR-142-5p, hsa-miR-320d, hsa-miR-320a, hsa-miR-320b and hsa-miR-17-5p miRNA may be used as a marker for detection of gastric cancer. The level of hsa-miR-484, hsa-miR-186-5p, hsa-miR-142-5p, hsa-miR-320d, hsa-miR-320a, hsa-miR-320b and hsa-miR-17-5p miRNA expression may be used as an indicator of cancer, in particular gastric cancer.

The level of hsa-miR-484, hsa-miR-186-5p, hsa-miR-142-5p, hsa-miR-320d, hsa-miR-320a, hsa-miR-320b and hsa-miR-17-5p miRNA expression may also be used as an indicator of likelihood of such a cancer. The level of expression of any one or more of these miRNAs may be detected for such a purpose. This may be combined optionally with detection of levels of hsa-miR-423-5p.

The expression levels of the miRNAs themselves may be detected. Alternatively, or in addition, the methods disclosed here may involve detection of a variant, homologue, derivative or fragment thereof such as a sequence having at least 75%, 80%, 85%, 90%, 95%, 96%, 97%, 98% or 99% sequence identity to the relevant miRNA.

In particular, gastric cancer may be detected where the expression level of certain miRNAs is increased, relative to persons known not to be suffering from gastric cancer.

For example, hsa-miR-484 in an individual is 0.39 or more, when measured as log₂(expression level of individual/expression level of control), than an individual known not to be suffering from gastric cancer, is indicative of gastric cancer (designated as “control”).

As another example, gastric cancer may also be detected where the expression level of hsa-miR-186-5p in an individual is 0.15 or more, when measured as log₂(expression level of individual/expression level of control), than an individual known not to be suffering from gastric cancer, is indicative of gastric cancer (designated as “control”).

As a further example, gastric cancer may be detected where the expression level of hsa-miR-320d in an individual is 0.48 or more, when measured as log₂(expression level of individual/expression level of control), than an individual known not to be suffering from gastric cancer, is indicative of gastric cancer (designated as “control”).

As yet another example, gastric cancer may be detected where the expression level of hsa-miR-320a in an individual is 0.49 or more, when measured as log₂(expression level of individual/expression level of control), than an individual known not to be suffering from gastric cancer, is indicative of gastric cancer (designated as “control”).

As yet another further example, gastric cancer may be detected where the expression level of hsa-miR-320b in an individual is 0.4 or more, when measured as log₂(expression level of individual/expression level of control), than an individual known not to be suffering from gastric cancer, is indicative of gastric cancer (designated as “control”).

Another example is where gastric cancer may be detected where the expression level of hsa-miR-423-5p in an individual is 0.54 or more, when measured as log₂(expression level of individual/expression level of control), than an individual known not to be suffering from gastric cancer, is indicative of gastric cancer (designated as “control”).

For other miRNAs, gastric cancer may be detected where the expression level of certain miRNAs is decreased, relative to persons known not to be suffering from gastric cancer.

For example, gastric cancer may be detected where the expression level of hsa-miR-142-5p in an individual is -0.35 or less, when measured as log₂(expression level of individual/expression level of control), than an individual known not to be suffering from gastric cancer, is indicative of gastric cancer (designated as “control”).

Another example is in which gastric cancer may be detected where the expression level of hsa-miR-17-5p in an individual is -0.37 or less, when measured as log₂(expression level of individual/expression level of control), than an individual known not to be suffering from gastric cancer, is indicative of gastric cancer (designated as “control”).

We therefore provide for methods of diagnosis or detection of a cancer, particularly gastric cancer. We further provide methods of diagnosis and detection of the aggressiveness or invasiveness or the metastatic state, or any combination of these, of such a cancer. The methods may comprise analysis of miRNA levels (e.g., by in situ hybridisation or RT-PCR). Such diagnostic and detection methods are described in further detail below.

### Extracellular Vesicles (EVS)

Extracellular vesicles (EVs) play an important role in cellular communication and promote tumour development¹³⁻¹⁶. miRNA expression in EVs is frequently dysregulated and is potentially useful for early diagnosis of cancer or other diseases¹⁵, ¹⁷⁻²⁵.

Since EVs are a key source of circulating miRNAs in blood serum, we hypothesized that isolation of EVs will enrich for miRNA biomarkers, leading to enhanced signal-to-noise ratios and thus improved diagnostic performance.

### Isolation of Extracellular Vesicles (EVS)

Extracellular vesicles such as exosomes may be isolated from a sample using any means known in the art.

The person skilled in the art will be aware of the various methods for isolation of extracellular vesicles from biological fluids that have been developed. Such methods may include centrifugation, chromatography, filtration, polymer-based precipitation and immunological separation.

One consideration when choosing an isolation method will be that of contamination of isolated extracellular vesicles with non-extracellular vesicle particles. This may cause wrong conclusions about biological activities of obtained extracellular vesicles and therefore should be avoided. Furthermore, the person skilled in the art will be aware that extracellular vesicles from different specimens can possess different protein/lipid and luminal contents and different sedimentation characteristics.

The following is adapted from Konstantin Yakimchuk (2015) Exosomes: Isolation and Characterization Methods and Specific Markers. Mater Methods 2015;5:1450.

Differential Centrifugation

Differential centrifugation consists of several centrifugation steps aiming to remove cells, large vesicles and debris and precipitate exosomes.

Differential centrifugation is the standard and very common method used to isolate exosomes from biological fluids and media. The efficiency of the method however is lower when viscous biological fluids such as plasma and serum are used for analysis.

Differential centrifugation remains one of the most common techniques of exosome isolation.

In detail, the method consists of several steps, including (1) low-speed centrifugation to remove cells and apoptotic debris, (2) higher speed spin to eliminate larger vesicles and finally, (3) a high-speed centrifugation to precipitate exosomes. The viscosity of the biofluids has a significant correlation with the purity of isolated exosomes. Moreover, biological samples with high viscosity require longer ultracentrifugation step and higher speed of centrifugation.

For example, exosomes may be purified from cultured cells in serum-free media with sequential centrifugation steps of 800×g and 2000×g and finally pelleted with an ultracentrifugation at 100,000×g.

Exosomes from primary cortical neurons may be obtained through sequential centrifugation of supernatants at 300×g for 10 min, at 2000×g for 10 min, 10,000×g for 30 min, and 100,000×g for 90 min at 4° C. and the last pellet was re-suspended and centrifuged again at 100,000×g for 90 min.

An protocol showing the use of ultracentrifugation for the isolation of exosomes is set out in the Examples as Example 3.

Density Gradient Centrifugation

Density gradient centrifugation combines ultracentrifugation with use of a sucrose density gradient. More specifically, density gradient centrifugation is used to separate exosomes from non-vesicular particles, such as proteins and protein/RNA aggregates. Thus, this method separates vesicles from the particles of different densities.

Use of an adequate centrifugation time is very important, otherwise contaminating particles may be still found in exosomal fractions if they possess similar densities. A density gradient flotation approach may be used to purify exosomes from blood preparations. Recent studies suggest the application of the exosomal pellet from ultracentrifugation to the sucrose gradient before performing centrifugation.

A protocol of a modified version of density gradient-based method, introduced as Cushioned Density Gradient Ultracentrifugation has recently been described. This method provides maximal recovery and high purity of the isolated exosomes and maintains their structure and functions.

Size-Exclusion Chromatography

Size-exclusion chromatography (SEC) is used to separate macromolecules on the base of size, not molecular weight.

The technique applies a column packed with porous polymeric beads containing multiple pores and tunnels. The molecules pass through the beads depending on their diameter. It takes a longer time for molecules with small radii to migrate through pores of the column, while macromolecules elute earlier from the column.

Size-exclusion chromatography allows precise separation of large and small molecules. Moreover, different eluting solutions can be applied to this method. Chromatography isolation has been shown to have more advantages compared to centrifugation methods, since the exosomes isolated by chromatography are not affected by shearing force, which can potentially change the structure of the vesicles. Currently, SEC is a widely accepted technique for isolation of exosomes present in both blood and urine.

In addition, a combination of SEC method with ultrafiltration has been used for isolation and analysis of urine-derived exosomes. Also, flow field-flow fractionation combined with a UV analyzer and light-scattering detector has been applied to analyze the size and pureness of the exosomes. The flow field-flow fractionation combines parabolic and cross-flow to isolate exosomes. The obtained exosomes have been detected by electron microscopy and mass spectrometry. In addition, a recent article by Lane et al (2017, Purification Protocols for Extracellular Vesicles. Methods Mol Biol. 2017;1660:111-130) has presented updated protocols for the purification of exosomes, including protocols for ultracentrifugation, SEC and density gradient centrifugation.

A protocol showing the use of column affinity-based purification for the isolation of exosomes is set out in the Examples as Example 5.

Filtration

Ultrafiltration membranes may also be used for isolation of exosomes. Depending on the size of microvesicles, this method allows the separation of exosomes from proteins and other macromolecules. Exosomes may also be isolated by trapping them via a porous structure.

Most common filtration membranes have pore sizes of 0.8 μm, 0.45 μm or 0.22 μm and may be used to collect exosomes larger than 800 nm, 400 nm or 200 nm. In particular, a micropillar porous silicon ciliated structure was designed to isolate 40-100 nm exosomes. During the initial step, the larger vesicles are removed. In the following step, the exosomal population is concentrated on the filtration membrane. The isolation step is relatively short, but the method requires pre-incubation of the silicon structure with PBS buffer. In the following step, the exosomal population is concentrated on the filtration membrane.

This method has not yet been tested using clinical samples. In addition to the standard filtration techniques, tangential flow filtration showed promising results for the effective isolation of exosomes and can be applied in both basic research and clinical analysis. This method is used for isolation of exosomes with well-determined size by removing free peptides and other small compounds. In addition, a combination of ultrafiltration with the SEC was shown to be very efficient for isolation of exosomes in in vitro studies and from adipose tissue.

Polymer-Based Precipitation

Polymer-based precipitation technique usually includes mixing the biological fluid with polymer-containing precipitation solution, incubation at 4 C and centrifugation at low speed.

One of the most common polymers used for polymer-based precipitation is polyethylene glycol (PEG). The precipitation with this polymer has a number of advantages, including mild effects on isolated exosomes and usage of neutral pH.

Several commercial kits applying PEG for isolation of exosomes are available, including ExoQuick™ (System Biosciences, Mountain View, Calif, USA). This kit is easy and fast to perform and there is no need for additional equipment. Recent studies demonstrated that the highest yield of exosomes was obtained using ultracentrifugation with ExoQuick™ method. However, contamination of exosomal isolates with non-exosomal materials remains a problem for polymer-based isolation methods. In addition, the polymer substance present in the isolate may interfere with downstream analysis.

A recent study by Niu et al has compared the application of ultracentrifugation, ultrafiltration and polymer-based precipitation for exosomal isolation from human endometrial cells and found that polymer-based method showed the lowest protein contamination.

Immunological Separation

Several techniques of immunological separation of exosomes have been developed. The immuno-chip method is based on surface exosomal receptors, which are used to isolate exosomes depending on their origin. Obtained exosomes are analyzed directly or used for DNA or total RNA isolation.

Exosomal intracellular proteins can be used as specific markers for isolation of exosomes. Antibody-coated magnetic beads were effectively used to isolate exosomes from antigen presenting cells. Also, exosomes of tumor origin were isolated from tumor cells using antibodies against tumor-associated HER2 and EpCAM. Isolated bead-exosome complexes can be analyzed by flow cytometry, Western blotting and electron microscopy. Moreover, Western blotting is applied to detect the exosome-specific proteins, including tetraspanins and the endosomal sorting complexes required for transport (ESCRT) proteins Alix and TSG101. However, the isolation using antibody-coated beads is not suitable for obtaining exosomes from large volumes.

In addition, ELISA-based ExoTEST™ was demonstrated to be effective for isolation of exosomes. Using ExoTEST™ plates coated with exosomal antibodies, exosomes can be isolated from various biological fluids. The method is applied for detection, analysis and quantification of both common and cell type-specific exosomal proteins.

A recent study has applied immunoaffinitive superparamagnetic nanoparticles (ISPN) to bind the exosomes. The researchers generated ISPNs by connecting anti-CD63 antibodies and nanoparticles and used them to isolate exosomes from body fluids.

A protocol showing the use of immunoaffinity affinity-based purification for the isolation of exosomes is set out in the Examples as Example 7.

Isolation by Sieving

This technique isolates exosomes by sieving them from biological liquids via a membrane and performing filtration by pressure or electrophoresis. The method requires a shorter separation period, but gives higher purity of isolated exosomes. This method is considered to be non-selective with regard to the specific types of exosomes. The only disadvantage of the sieving separation is the low recovery of isolated exosomes.

Ultracentrifugation (UC)

Ultracentrifugation (UC) is the current gold standard for EV isolation. However, it may not be suitable for use in clinical settings as the procedure is time-consuming, low-throughput and is highly variable among different operators²⁶⁻²⁹.

There are other isolation methods available but these appear to isolate different subtypes of EV, making comparisons difficult²⁸, ³⁰⁻³⁶.

In this study we systematically compared the EV-associated miRNA (EV-miRNA) recovery performance from commercially available EV isolation kit/reagents with two objectives: (1) to identify a robust EV isolation method that is suitable for serum EV-miRNA recovery in clinical settings; and (2) to identify serum EV-miRNA biomarkers that can be used for non-invasive detection of gastric cancer (GC).

An protocol showing the use of ultracentrifugation for the isolation of exosomes is set out in the Examples as Example 3.

### Isolation of Exosomes Using Polymer-Based Precipitation

In the methods disclosed here, extracellular vesicles such as exosomes may be isolated using polymer-based precipitation. For this purpose, a commercially available kit such as Invitrogen Total Exosome Isolation Reagent (from serum) (Catalog number: 4478360, ThermoFisher Scientific, USA) may be used.

The following example protocol may be used to isolate exosomes using polymer-based precipitation. A further protocol showing the use of polymer-based precipitation for the isolation of exosomes is set out in the Examples as Example 4.

Protocol for Polymer-Based Precipitation

Prepare Sample

1. Remove the serum sample from storage and place it on ice. If the sample is frozen, thaw the sample in a 25° C. water bath until it is completely liquid, and place on ice until needed.

2. Centrifuge the serum sample at 2000×g for 30 minutes to remove cells and debris.

3. Transfer the supernatant containing the clarified serum to a new tube without disturbing the pellet, and place it on ice until ready to perform the isolation.

Isolate Exosomes

1. Transfer the required volume of clarified serum to a new tube and add 0.2 volumes of the Total Exosome Isolation (from serum) reagent.

2. Mix the serum/reagent mixture well either by vortexing or pipetting up and down until there is a homogenous solution.

Note: The solution should have a cloudy appearance.

3. Incubate the sample at 2° C. to 8° C. for 30 minutes.

4. After incubation, centrifuge the sample at 10,000×g for 10 minutes at room temperature.

5. Aspirate and discard the supernatant. Exosomes are contained in the pellet at the bottom of the tube.

6. Use a pipette tip to completely resuspend the pellet in a convenient volume of 1×PBS or similar buffer.

For a starting serum volume of 100 μL, a resuspension volume of 25-50 μL should be used. For a starting serum volume of 1 mL , a resuspension volume of 100-500 μL should be used.

7. Once the pellet is resuspended, the exosomes are ready for downstream analysis or further purification through affinity methods.

Keep isolated exosomes at 2° C. to 8° C. for up to 1 week, or at ≤20° C. for long-term storage.

### Micrornas (MIRNAS)

MicroRNAs (miRNAs) are small non-coding RNAs (˜19-22 nucleotides) that regulate protein expression and exert physiological significance in several key cellular processes, such as cell differentiation, proliferation and apoptosis¹, ². Circulating miRNAs, which can be readily detected in biofluids such as serum, plasma or whole blood, are promising liquid biopsy biomarkers for non-invasive detection of various diseases, including cancer. In addition, aberrations affecting miRNAs have been shown to significantly affect cancer genesis and progression³⁻⁶. Due to their stability in serum/plasma, substantial attention and tremendous efforts have been dedicated to identify miRNA biomarkers for early detection, prognosis or therapeutic purposes⁷⁻⁹. However, changes in the miRNA expression level might be subtle during the onset of disease, thus making diagnosis challenging¹⁰⁻¹². An ideal liquid biopsy biomarker should have a high signal-to-noise ratio between cancer and control samples, which can be readily detectable in clinical settings.

### HSA-MIR-484, HSA-MIR-186-5P, HSA-MIR-142-5P, HSA-MIR-320D, HSA-MIR-320A, HSA-MIR-320B, HSA-MIR-17-5P and HSA-MIR-423-5P MIRNAS

The methods and compositions described here may make use of hsa-miR-484, hsa-miR-186-5p, hsa-miR-142-5p, hsa-miR-320d, hsa-miR-320a, hsa-miR-320b, hsa-miR-17-5p and optionally hsa-miR-423-5p miRNAs, as well as variants, homologues, derivatives and fragments of any of these, for the diagnosis, detection of susceptibility to, treatment, alleviation or prophylaxis of gastric cancer in an individual.

The terms “hsa-miR-484, hsa-miR-186-5p, hsa-miR-142-5p, hsa-miR-320d, hsa-miR-320a, hsa-miR-320b and hsa-miR-17-5p miRNAs” and “hsa-miR-484, hsa-miR-186-5p, hsa-miR-142-5p, hsa-miR-320d, hsa-miR-320a, hsa-miR-320b and hsa-miR-17-5p nucleic acid” may be used interchangeably. Similarly, the term “hsa-miR-423-5p miRNA” and “hsa-miR-423-5p nucleic acid” may be used interchangeably.

These terms are also intended to include a nucleic acid sequence capable of encoding an hsa-miR-484, hsa-miR-186-5p, hsa-miR-142-5p, hsa-miR-320d, hsa-miR-320a, hsa-miR-320b or hsa-miR-17-5p miRNA (or and hsa-miR-423-5p where this is used) and/or a fragment, derivative, homologue or variant of this. These terms are also intended to include a nucleic acid sequence which is a fragment, derivative, homologue or variant of an hsa-miR-484, hsa-miR-186-5p, hsa-miR-142-5p, hsa-miR-320d, hsa-miR-320a, hsa-miR-320b or hsa-miR-17-5p (or and hsa-miR-423-5p where this is employed) polynucleotide having a specific sequence disclosed in this document.

Where reference is made to an hsa-miR-484, hsa-miR-186-5p, hsa-miR-142-5p, hsa-miR-320d, hsa-miR-320a, hsa-miR-320b, hsa-miR-17-5p miRNA (or and hsa-miR-423-5p) nucleic acid, this should be taken as a reference to a nucleic acid sequence capable of encoding such an miRNA. Such miRNAs may comprise one or more biological activities of a native hsa-miR-484, hsa-miR-186-5p, hsa-miR-142-5p, hsa-miR-320d, hsa-miR-320a, hsa-miR-320b or hsa-miR-17-5p miRNA (or and hsa-miR-423-5p where this is relevant), as the case may be.

hsa-miR-484, hsa-miR-186-5p, hsa-miR-142-5p, hsa-miR-320d, hsa-miR-320a, hsa-miR-320b, hsa-miR-17-5p miRNAs and optionally hsa-miR-423-5p may be used for a variety of means, as described in this document. For example, hsa-miR-484, hsa-miR-186-5p, hsa-miR-142-5p, hsa-miR-320d, hsa-miR-320a, hsa-miR-320b, hsa-miR-17-5p miRNAs or hsa-miR-423-5 miRNA may be used treat an individual suffering from, or suspected to be suffering from gastric cancer, or to prevent such a condition or to alleviate any symptoms arising as a result of such a condition. Other uses will be evident to the skilled reader, and are also encompassed in this document.

The term “polynucleotide”, as used in this document, generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

It will be understood by the skilled person that numerous nucleotide sequences can encode the same polypeptide as a result of the degeneracy of the genetic code.

As used herein, the term “nucleotide sequence” refers to nucleotide sequences, oligonucleotide sequences, polynucleotide sequences and variants, homologues, fragments and derivatives thereof (such as portions thereof). The nucleotide sequence may be DNA or RNA of genomic or synthetic or recombinant origin which may be double-stranded or single-stranded whether representing the sense or antisense strand or combinations thereof. The term nucleotide sequence may be prepared by use of recombinant DNA techniques (for example, recombinant DNA).

The term “nucleotide sequence” may mean DNA or RNA.

Other Nucleic Acids

We also provide nucleic acids which are fragments, homologues, variants or derivatives of hsa-miR-484, hsa-miR-186-5p, hsa-miR-142-5p, hsa-miR-320d, hsa-miR-320a, hsa-miR-320b and hsa-miR-17-5p miRNAs, as well as and hsa-miR-423-5p miRNA where this is optionally used.

The terms “variant”, “homologue”, “derivative” or “fragment” in relation to hsa-miR-484, hsa-miR-186-5p, hsa-miR-142-5p, hsa-miR-320d, hsa-miR-320a, hsa-miR-320b, hsa-miR-17-5p and hsa-miR-423-5p miRNAs include any substitution of, variation of, modification of, replacement of, deletion of or addition of one (or more) nucleic acids from or to the sequence of an hsa-miR-484, hsa-miR-186-5p, hsa-miR-142-5p, hsa-miR-320d, hsa-miR-320a, hsa-miR-320b, hsa-miR-17-5p and hsa-miR-423-5p miRNAs. Unless the context admits otherwise, references to “hsa-miR-484, hsa-miR-186-5p, hsa-miR-142-5p, hsa-miR-320d, hsa-miR-320a, hsa-miR-320b and hsa-miR-17-5p miRNAs” and “hsa-miR-484, hsa-miR-186-5p, hsa-miR-142-5p, hsa-miR-320d, hsa-miR-320a, hsa-miR-320b and hsa-miR-17-5p nucleic acid”, “hsa-miR-484, hsa-miR-186-5p, hsa-miR-142-5p, hsa-miR-320d, hsa-miR-320a, hsa-miR-320b and hsa-miR-17-5p nucleotide sequence” etc include references to such variants, homologues, derivatives and fragments of hsa-miR-484, hsa-miR-186-5p, hsa-miR-142-5p, hsa-miR-320d, hsa-miR-320a, hsa-miR-320b or hsa-miR-17-5p miRNAs. The same applies for hsa-miR-423-5p.

The nucleotide sequence may encode a polypeptide having any one or more hsa-miR-484, hsa-miR-186-5p, hsa-miR-142-5p, hsa-miR-320d, hsa-miR-320a, hsa-miR-320b or hsa-miR-17-5p miRNA activity (or hsa-miR-423-5p activity, where relevant). The term “homologue” may be intended to cover identity with respect to structure and/or function such that the resultant nucleotide sequence encodes a polypeptide which has hsa-miR-484, hsa-miR-186-5p, hsa-miR-142-5p, hsa-miR-320d, hsa-miR-320a, hsa-miR-320b or hsa-miR-17-5p miRNA activity (or hsa-miR-423-5p activity). For example, a homologue etc of hsa-miR-484, hsa-miR-186-5p, hsa-miR-142-5p, hsa-miR-320d, hsa-miR-320a, hsa-miR-320b, hsa-miR-17-5p miRNA or hsa-miR-423-5p may have an increased or decreased expression level in cells from an individual suffering from gastric cancer compared to normal cells. With respect to sequence identity (i.e. similarity), there may be at least 70%, at least 75%, at least 85% or at least 90% sequence identity. There may be at least 95%, such as at least 98%, sequence identity to a relevant sequence such as any nucleic acid sequence of hsa-miR-484, hsa-miR-186-5p, hsa-miR-142-5p, hsa-miR-320d, hsa-miR-320a, hsa-miR-320b or hsa-miR-17-5p miRNA. These terms also encompass allelic variations of the sequences.

Variants, Derivatives and Homologues

hsa-miR-484, hsa-miR-186-5p, hsa-miR-142-5p, hsa-miR-320d, hsa-miR-320a, hsa-miR-320b and hsa-miR-17-5p miRNA (and hsa-miR-423-5p miRNA where used optionally) nucleic acid variants, fragments, derivatives and homologues may comprise RNA. They may be single-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of this document, it is to be understood that the polynucleotides may be modified by any method available in the art. Such modifications may be carried out in order to enhance the in vivo activity or life span of polynucleotides of interest.

Where the polynucleotide is double-stranded, both strands of the duplex, either individually or in combination, are encompassed by the methods and compositions described here. Where the polynucleotide is single-stranded, it is to be understood that the complementary sequence of that polynucleotide is also included.

The terms “variant”, “homologue” or “derivative” in relation to a nucleotide sequence include any substitution of, variation of, modification of, replacement of, deletion of or addition of one (or more) nucleic acid from or to the sequence. Said variant, homologues or derivatives may code for a polypeptide having biological activity. Such fragments, homologues, variants and derivatives of hsa-miR-484, hsa-miR-186-5p, hsa-miR-142-5p, hsa-miR-320d, hsa-miR-320a, hsa-miR-320b, hsa-miR-17-5p miRNAs or hsa-miR-423-5p may comprise modulated activity, as set out above.

As indicated above, with respect to sequence identity, a “homologue” may have at least 5% identity, at least 10% identity, at least 15% identity, at least 20% identity, at least 25% identity, at least 30% identity, at least 35% identity, at least 40% identity, at least 45% identity, at least 50% identity, at least 55% identity, at least 60% identity, at least 65% identity, at least 70% identity, at least 75% identity, at least 80% identity, at least 85% identity, at least 90% identity, or at least 95% identity to the relevant sequence, such as any nucleic acid sequence of a hsa-miR-484, hsa-miR-186-5p, hsa-miR-142-5p, hsa-miR-320d, hsa-miR-320a, hsa-miR-320b or hsa-miR-17-5p miRNA.

There may be at least 95% identity, at least 96% identity, at least 97% identity, at least 98% identity or at least 99% identity. Nucleotide identity comparisons may be conducted as described above. A sequence comparison program which may be used is the GCG Wisconsin Bestfit program described above. The default scoring matrix has a match value of 10 for each identical nucleotide and -9 for each mismatch. The default gap creation penalty is -50 and the default gap extension penalty is -3 for each nucleotide.

Hybridisation

We further describe nucleotide sequences that are capable of hybridising selectively to any of the sequences presented herein, or any variant, fragment or derivative thereof, or to the complement of any of the above. Nucleotide sequences may be at least 5, 10, or 15 nucleotides in length, such as at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Polynucleotides capable of selectively hybridising to the nucleotide sequences presented herein, or to their complement, may be at least 40% homologous, at least 45% homologous, at least 50% homologous, at least 55% homologous, at least 60% homologous, at least 65% homologous, at least 70% homologous, at least 75% homologous, at least 80% homologous, at least 85% homologous, at least 90% homologous, or at least 95% homologous to the corresponding nucleotide sequences presented herein, such as any nucleic acid sequence of a hsa-miR-484, hsa-miR-186-5p, hsa-miR-142-5p, hsa-miR-320d, hsa-miR-320a, hsa-miR-320b or hsa-miR-17-5p miRNA (or hsa-miR-423-5p, as optional). Such polynucleotides may be generally at least 70%, at least 80 or 90% or at least 95% or 98% homologous to the corresponding nucleotide sequences over a region of at least 5, 10, 15 or 20, such as at least 25 or 30, for instance at least 40, 60 or 100 or more contiguous nucleotides.

The term “selectively hybridizable” means that the polynucleotide used as a probe is used under conditions where a target polynucleotide is found to hybridize to the probe at a level significantly above background. The background hybridization may occur because of other polynucleotides present, for example, in the cDNA or genomic DNA library being screened. In this event, background implies a level of signal generated by interaction between the probe and a non-specific DNA member of the library which is less than 10 fold, such as less than 100 fold as intense as the specific interaction observed with the target DNA. The intensity of interaction may be measured, for example, by radiolabelling the probe, e.g. with ³²P or ³³P or with non-radioactive probes (e.g., fluorescent dyes, biotin or digoxigenin).

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained elsewhere in this document.

Maximum stringency typically occurs at about Tm−5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

We provide nucleotide sequences that may be able to hybridise to the hsa-miR-484, hsa-miR-186-5p, hsa-miR-142-5p, hsa-miR-320d, hsa-miR-320a, hsa-miR-320b or hsa-miR-17-5p miRNA (and hsa-miR-423-5p miRNA where used) nucleic acids, fragments, variants, homologues or derivatives under stringent conditions (e.g. 65° C. and 0.1xSSC (1xSSC =0.15 M NaCl, 0.015 M Na₃ Citrate pH 7.0)).

Generation of Homologues, Variants and Derivatives

Polynucleotides which are not 100% identical to the relevant sequences (hsa-miR-484, hsa-miR-186-5p, hsa-miR-142-5p, hsa-miR-320d, hsa-miR-320a, hsa-miR-320b, hsa-miR-17-5p and hsa-miR-423-5p miRNAs) but which are also included, as well as homologues, variants and derivatives of hsa-miR-484, hsa-miR-186-5p, hsa-miR-142-5p, hsa-miR-320d, hsa-miR-320a, hsa-miR-320b, hsa-miR-17-5p and hsa-miR-423-5p miRNAs can be obtained in a number of ways. Other variants of the sequences may be obtained for example by probing RNA libraries made from a range of individuals, for example individuals from different populations. For example, hsa-miR-484, hsa-miR-186-5p, hsa-miR-142-5p, hsa-miR-320d, hsa-miR-320a, hsa-miR-320b, hsa-miR-17-5p miRNA and hsa-miR-423-5p homologues may be identified from other individuals, or other species. Further recombinant hsa-miR-484, hsa-miR-186-5p, hsa-miR-142-5p, hsa-miR-320d, hsa-miR-320a, hsa-miR-320b, hsa-miR-17-5p and hsa-miR-423-5p miRNA nucleic acids and polypeptides may be produced by identifying corresponding positions in the homologues, and synthesising or producing the molecule as described elsewhere in this document.

In addition, other viral/bacterial, or cellular homologues of hsa-miR-484, hsa-miR-186-5p, hsa-miR-142-5p, hsa-miR-320d, hsa-miR-320a, hsa-miR-320b, hsa-miR-17-5p and hsa-miR-423-5p miRNAs, particularly cellular homologues found in mammalian cells (e.g. rat, mouse, bovine and primate cells), may be obtained and such homologues and fragments thereof in general will be capable of selectively hybridising to human hsa-miR-484, hsa-miR-186-5p, hsa-miR-142-5p, hsa-miR-320d, hsa-miR-320a, hsa-miR-320b, hsa-miR-17-5p and hsa-miR-423-5p miRNAs. Such homologues may be used to design non-human hsa-miR-484, hsa-miR-186-5p, hsa-miR-142-5p, hsa-miR-320d, hsa-miR-320a, hsa-miR-320b, hsa-miR-17-5p and hsa-miR-423-5p miRNA nucleic acids, fragments, variants and homologues. Mutagenesis may be carried out by means known in the art to produce further variety.

Sequences of hsa-miR-484, hsa-miR-186-5p, hsa-miR-142-5p, hsa-miR-320d, hsa-miR-320a, hsa-miR-320b, hsa-miR-17-5p and hsa-miR-423-5p miRNA homologues may be obtained by probing libraries made from other animal species, and probing such libraries with probes comprising all or part of any of the hsa-miR-484, hsa-miR-186-5p, hsa-miR-142-5p, hsa-miR-320d, hsa-miR-320a, hsa-miR-320b, hsa-miR-17-5p and hsa-miR-423-5p miRNA nucleic acids, fragments, variants and homologues, or other fragments of hsa-miR-484, hsa-miR-186-5p, hsa-miR-142-5p, hsa-miR-320d, hsa-miR-320a, hsa-miR-320b, hsa-miR-17-5p or hsa-miR-423-5p miRNA under conditions of medium to high stringency.

Similar considerations apply to obtaining species homologues and allelic variants of the polypeptide or nucleotide sequences disclosed here.

Variants and strain/species homologues may also be obtained using degenerate PCR which will use primers designed to target sequences within the variants and homologues encoding conserved amino acid sequences within the sequences of the hsa-miR-484, hsa-miR-186-5p, hsa-miR-142-5p, hsa-miR-320d, hsa-miR-320a, hsa-miR-320b, hsa-miR-17-5p or hsa-miR-423-5p miRNA nucleic acids. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences. It will be appreciated by the skilled person that overall nucleotide homology between sequences from distantly related organisms is likely to be very low and thus in these situations degenerate PCR may be the method of choice rather than screening libraries with labelled fragments the hsa-miR-484, hsa-miR-186-5p, hsa-miR-142-5p, hsa-miR-320d, hsa-miR-320a, hsa-miR-320b, hsa-miR-17-5p miRNA or hsa-miR-423-5p sequences.

In addition, homologous sequences may be identified by searching nucleotide and/or protein databases using search algorithms such as the BLAST suite of programs.

Alternatively, such polynucleotides may be obtained by site directed mutagenesis of characterised sequences, for example, hsa-miR-484, hsa-miR-186-5p, hsa-miR-142-5p, hsa-miR-320d, hsa-miR-320a, hsa-miR-320b, hsa-miR-17-5p or hsa-miR-423-5p miRNA nucleic acids, or variants, homologues, derivatives or fragments thereof. This may be useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

The polynucleotides described here may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 8, 9, 10, or 15, such as at least 20, for example at least 25, 30 or 40 nucleotides in length, and are also encompassed by the term “polynucleotides” as used herein.

Polynucleotides such as a DNA polynucleotides and probes may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Primers comprising fragments of hsa-miR-484, hsa-miR-186-5p, hsa-miR-142-5p, hsa-miR-320d, hsa-miR-320a, hsa-miR-320b, hsa-miR-17-5p and optionally hsa-miR-423-5p miRNA are particularly useful in the methods of detection of hsa-miR-484, hsa-miR-186-5p, hsa-miR-142-5p, hsa-miR-320d, hsa-miR-320a, hsa-miR-320b, hsa-miR-17-5p or hsa-miR-423-5p miRNA expression, such as up-regulation or down-regulation of hsa-miR-484, hsa-miR-186-5p, hsa-miR-142-5p, hsa-miR-320d, hsa-miR-320a, hsa-miR-320b, hsa-miR-17-5p and hsa-miR-423-5p miRNA expression, for example, as associated with gastric cancer. Suitable primers for amplification of hsa-miR-484, hsa-miR-186-5p, hsa-miR-142-5p, hsa-miR-320d, hsa-miR-320a, hsa-miR-320b, hsa-miR-17-5p or hsa-miR-423-5p miRNA may be generated from any suitable stretch of hsa-miR-484, hsa-miR-186-5p, hsa-miR-142-5p, hsa-miR-320d, hsa-miR-320a, hsa-miR-320b, hsa-miR-17-5p or hsa-miR-423-5p miRNA. Primers which may be used include those capable of amplifying a sequence of hsa-miR-484, hsa-miR-186-5p, hsa-miR-142-5p, hsa-miR-320d, hsa-miR-320a, hsa-miR-320b, hsa-miR-17-5p or hsa-miR-423-5p miRNA which is specific.

Although hsa-miR-484, hsa-miR-186-5p, hsa-miR-142-5p, hsa-miR-320d, hsa-miR-320a, hsa-miR-320b, hsa-miR-17-5p and hsa-miR-423-5p miRNA primers may be provided on their own, they are most usefully provided as primer pairs, comprising a forward primer and a reverse primer.

Longer polynucleotides will generally be produced using recombinant means, for example using a PCR (polymerase chain reaction) cloning techniques. This will involve making a pair of primers (e.g. of about 15 to 30 nucleotides), bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Polynucleotides or primers may carry a revealing label. Suitable labels include radioisotopes such as ³²P or ³⁵S, digoxigenin, fluorescent dyes, enzyme labels, or other protein labels such as biotin. Such labels may be added to polynucleotides or primers and may be detected using by techniques known per se. Polynucleotides or primers or fragments thereof labelled or unlabeled may be used by a person skilled in the art in nucleic acid-based tests for detecting or sequencing polynucleotides in the human or animal body.

Such tests for detecting generally comprise bringing a biological sample containing DNA or RNA into contact with a probe comprising a polynucleotide or primer under hybridising conditions and detecting any duplex formed between the probe and nucleic acid in the sample. Such detection may be achieved using techniques such as PCR or by immobilising the probe on a solid support, removing nucleic acid in the sample which is not hybridised to the probe, and then detecting nucleic acid which has hybridised to the probe. Alternatively, the sample nucleic acid may be immobilised on a solid support, and the amount of probe bound to such a support can be detected. Suitable assay methods of this and other formats can be found in for example WO89/03891 and WO90/13667.

Tests for sequencing nucleotides, for example, the hsa-miR-484, hsa-miR-186-5p, hsa-miR-142-5p, hsa-miR-320d, hsa-miR-320a, hsa-miR-320b, hsa-miR-17-5p and hsa-miR-423-5p miRNA nucleic acids, involve bringing a biological sample containing target DNA or RNA into contact with a probe comprising a polynucleotide or primer under hybridising conditions and determining the sequence by, for example the Sanger dideoxy chain termination method (see Sambrook et al.).

Such a method generally comprises elongating, in the presence of suitable reagents, the primer by synthesis of a strand complementary to the target DNA or RNA and selectively terminating the elongation reaction at one or more of an A, C, G or T/U residue; allowing strand elongation and termination reaction to occur; separating out according to size the elongated products to determine the sequence of the nucleotides at which selective termination has occurred. Suitable reagents include a DNA polymerase enzyme, the deoxynucleotides dATP, dCTP, dGTP and dTTP, a buffer and ATP. Dideoxynucleotides are used for selective termination.

### Isolation of Mirnas

miRNAs may be isolated from exosomes using any means known in the art.

The person skilled in the art will be aware of the various methods for isolation of miRNAs from biological fluids that have been developed. Commercially available miRNA isolation kits are available, for example from miRNeasy kit (Qiagen, Calif.), the miRVana PARIS kit (Ambion, Tex.), and the total RNA isolation kit (Norgen Biotek, Canada). Any of these may be used to isolate miRNAs from a sample.

The following example protocol, from the miRNeasy Serum/Plasma Handbook (QIAGEN, February 2012), may be used to isolate miRNA using the miRNeasy kit:

1. Prepare serum or plasma or thaw frozen samples.

2. Add 5 volumes QIAzol Lysis Reagent (see Table 2 for guidelines). Mix by vortexing or pipetting up and down.

| | | | Protocol | |
|---|---|---|---|---|
| | | | step 7: | |
| | Protocol | | approx.. | |
| | step 2: | | volume of | Protocol |
| | QIAzol | Protocol | upper | step 8: |
| Serum/ | Lysis | step 5: | aqueous | 100% |
| plasma | Reagent | chloroform | phase | ethanol |
| (μl) | (μl) | (μl) | (μl) | (μl) |
| ≤50 | 250 | 50 | 150 | 225 |
| 100 | 500 | 100 | 300 | 450 |
| 200 | 1000 | 200 | 600 | 900 |

Note: If the volume of plasma or serum is not limited, we recommend using 100-200 μl per RNA preparation.

Note: After addition of QIAzol Lysis Reagent, lysates can be stored at −70° C for several months.

3. Place the tube containing the lysate on the benchtop at room temperature (15-25° C.) for 5 min.

4. Add 3.5 μl miRNeasy Serum/Plasma Spike-In Control (1.6×10⁸ copies/μl working solution) and mix thoroughly.

For details on making appropriate stocks and working solutions of miRNeasy Serum/Plasma Spike-In Control, see Appendix B, page 25

5. Add chloroform of an equal volume to the starting sample to the tube containing the lysate and cap it securely (see Table 2 for guidelines). Vortex or shake vigorously for 15 s.

Thorough mixing is important for subsequent phase separation.

6. Place the tube containing the lysate on the benchtop at room temperature (15-25° C.) for 2-3 min.

7. Centrifuge for 15 min at 12,000×g at 4° C. After centrifugation, heat the centrifuge up to room temperature (15-25° C.) if the same centrifuge will be used for the next centrifugation steps.

After centrifugation, the sample separates into 3 phases: an upper, colorless, aqueous phase containing RNA; a white interpose; and a lower, red, organic phase. See Table 2 for the approximate volume of the aqueous phase.

8. Transfer the upper aqueous phase to a new collection tube (not supplied). Avoid transfer of any interphase material. Add 1.5 volumes of 100% ethanol and mix thoroughly by pipetting up and down several times. Do not centrifuge. Continue without delay with step 9.

A precipitate may form after addition of ethanol, but this will not affect the procedure.

9. Pipet up to 700 pl of the sample, including any precipitate that may have formed, into an RNeasy MinElute spin column in a 2 ml collection tube (supplied). Close the lid gently and centrifuge at ≥8000×g (≥10,000 rpm) for 15 s at room temperature (15-25° C.). Discard the flow-through.*

Reuse the collection tube in step 10.

10. Repeat step 9 using the remainder of the sample. Discard the flow-through.*

Reuse the collection tube in step 11.

11. Add 700 μl Buffer RWT to the RNeasy MinElute spin column. Close the lid gently and centrifuge for 15 s at ≥8000×g (10,000 rpm) to wash the column. Discard the flow-through.*

Reuse the collection tube in step 12.

12. Pipet 500 μl Buffer RPE onto the RNeasy MinElute spin column. Close the lid gently and centrifuge for 15 s at ≥8000×g (≥10,000 rpm) to wash the column. Discard the flow-through.

Reuse the collection tube in step 13.

13. Pipet 500 μl of 80% ethanol onto the RNeasy MinElute spin column. Close the lid gently and centrifuge for 2 min at ≥8000×g (≥10,000 rpm) to wash the spin column membrane. Discard the collection tube with the flow-through.

Note: 80% ethanol should be prepared with ethanol (96-100%) and RNase-free water.

Note: After centrifugation, carefully remove the RNeasy MinElute spin column from the collection tube so that the column does not contact the flow-through. Otherwise, carryover of ethanol will occur.

14. Place the RNeasy MinElute spin column into a new 2 ml collection tube (supplied). Open the lid of the spin column, and centrifuge at full speed for 5 min to dry the membrane. Discard the collection tube with the flow-through.

To avoid damage to their lids, place the spin columns into the centrifuge with at least one empty position between columns. Orient the lids so that they point in a direction opposite to the rotation of the rotor (e.g., if the rotor rotates clockwise, orient the lids counterclockwise).

It is important to dry the spin column membrane, since residual ethanol may interfere with downstream reactions. Centrifugation with the lids open ensures that no ethanol is carried over during RNA elution.

15. Place the RNeasy MinElute spin column in a new 1.5 ml collection tube (supplied). Add 14 μl RNase-free water directly to the center of the spin column membrane. Close the lid gently, and centrifuge for 1 min at full speed to elute the RNA.

As little as 10 μl RNase-free water can be used for elution if a higher RNA concentration is required, but the yield will be reduced by approximately 20%. Do not elute with less than 10 μl RNase-free water, as the spin column membrane will not be sufficiently hydrated.

The dead volume of the RNeasy MinElute spin column is 2 pl: elution with 14 pl RNase-free water results in a 12 μl eluate.

### Gastric Cancer

Gastric cancer is also known as stomach cancer.

Information about gastric cancer is published by the American Cancer Society and may be obtained from https://www.cancer.org/cancer/stomach-cancer

Gastric cancer is described in detail in the following documents:

Lello et al., 2007, Short and long-term survival from gastric cancer. A population-based study from a county hospital during 25 years. Acta Oncologica 46:3, 308-315.

Sanjeevaiah A, Cheedella N, Hester C, Porembka MR. Gastric Cancer: Recent Molecular Classification Advances, Racial Disparity, and Management Implications. J Oncol Pract. 2018 Apr;14(4): 217-224. doi: 10.1200/JOP.17.00025.

Rawla P, Barsouk A. Epidemiology of gastric cancer: global trends, risk factors and prevention. Prz Gastroenterol. 2019;14(1): 26-38. doi: 10.5114/pg.2018.80001. Epub 2018 Nov 28.

Pasechnikov V, Chukov S, Fedorov E, Kikuste I, Leja M. Gastric cancer: prevention, screening and early diagnosis. World J Gastroenterol. 2014;20(38): 13842-13862. doi: 10.3748/wjg.v20.i38.13842

Kim G H, Liang P S, Bang S J, Hwang J H. Screening and surveillance for gastric cancer in the United States: Is it needed? Gastrointest Endosc. 2016 Jul;84(1): 18-28. doi: 10.1016/j.gie.2016.02.028. Epub 2016 Mar 3.

### Gastric Cancer Risk Factors

The following is adapted from the American Cancer Society.

Risk factors for gastric cancer include the following:

Gender

Stomach cancer is more common in men than in women.

Age

There is a sharp increase in stomach cancer rates in people over age 50. Most people diagnosed with stomach cancer are between their late 60s and 80s.

Ethnicity

In the United States, stomach cancer is more common in Hispanic Americans, African Americans, Native Americans, and Asian/Pacific Islanders than it is in non-Hispanic whites.

Geography

Worldwide, stomach cancer is more common in Japan, China, Southern and Eastern Europe, and South and Central America. This disease is less common in Northern and Western Africa, South Central Asia, and North America.

Helicobacter pylori Infection

Infection with Helicobacter pylori (H pylon) bacteria seems to be a major cause of stomach cancer, especially cancers in the lower (distal) part of the stomach. Long-term infection of the stomach with this germ may lead to inflammation (called chronic atrophic gastritis) and pre-cancerous changes of the inner lining of the stomach.

People with stomach cancer have a higher rate of H pylori infection than people without this cancer. H pylori infection is also linked to some types of lymphoma of the stomach. Even so, most people who carry this germ in their stomach never develop cancer.

Stomach Lymphoma

People who have had a certain type of lymphoma of the stomach known as mucosa-associated lymphoid tissue (MALT) lymphoma have an increased risk of getting adenocarcinoma of the stomach. This is probably because MALT lymphoma of the stomach is caused by infection with H pylori bacteria.

Diet

An increased risk of stomach cancer is seen in people with diets that have large amounts of smoked foods, salted fish and meat, and pickled vegetables. Nitrates and nitrites are substances commonly found in cured meats. They can be converted by certain bacteria, such as H pylori, into compounds that have been shown to cause stomach cancer in lab animals.

On the other hand, eating lots of fresh fruits and vegetables appears to lower the risk of stomach cancer.

Tobacco Use

Smoking increases stomach cancer risk, particularly for cancers of the upper portion of the stomach near the oesophagus. The rate of stomach cancer is about doubled in smokers.

Being Overweight or Obese

Being overweight or obese is a possible cause of cancers of the cardia (the upper part of the stomach nearest the oesophagus), but the strength of this link is not yet clear.

Previous Stomach Surgery

Stomach cancers are more likely to develop in people who have had part of their stomach removed to treat non-cancerous diseases such as ulcers. This might be because the stomach makes less acid, which allows more nitrite-producing bacteria to be present. Reflux (backup) of bile from the small intestine into the stomach after surgery might also add to the increased risk. These cancers typically develop many years after the surgery.

Pernicious Anaemia

Certain cells in the stomach lining normally make a substance called intrinsic factor (IF) that we need to absorb vitamin B12 from foods. People without enough IF may end up with a vitamin B12 deficiency, which affects the body's ability to make new red blood cells and can cause other problems as well. This condition is called pernicious anaemia. Along with anaemia (too few red blood cells), people with this disease have an increased risk of stomach cancer.

Menetrier Disease (Hypertrophic Gastropathy)

In this condition, excess growth of the stomach lining causes large folds in the lining and leads to low levels of stomach acid. Because this disease is very rare, it is not known exactly how much this increases the risk of stomach cancer.

Type A Blood

Blood type groups refer to certain substances that are normally present on the surface of red blood cells and some other types of cells. These groups are important in matching blood for transfusions. For unknown reasons, people with type A blood have a higher risk of getting stomach cancer.

Inherited Cancer Syndromes

Some inherited conditions may raise a person's risk of stomach cancer.

Hereditary Diffuse Gastric Cancer

This inherited syndrome greatly increases the risk of developing stomach cancer. This condition is rare, but the lifetime stomach cancer risk among affected people is about 70% to 80%. Women with this syndrome also have an increased risk of getting a certain type of breast cancer. This condition is caused by mutations (defects) in the CDH1 gene.

Lynch Syndrome or Hereditary Non-Polyposis Colorectal Cancer (HNPCC)

Lynch syndrome (formerly known as HNPCC) is an inherited genetic disorder that increases the risk of colorectal cancer, stomach cancer, and some other cancers. In most cases, this disorder is caused by a defect in either the MLH1 or MSH2 gene, but other genes can cause Lynch syndrome, including MLH3, MSH6, TGFBR2, PMS1, and PMS2.

Familial Adenomatous Polyposis (FAP)

In FAP, people get many polyps in the colon, and sometimes in the stomach and intestines as well. People with this syndrome are at greatly increased risk of getting colorectal cancer and have a slightly increased risk of getting stomach cancer. It is caused by mutations in the APC gene.

BRCA1 and BRCA2

People who carry mutations of the inherited breast cancer genes BRCA1 or BRCA2 may also have a higher rate of stomach cancer.

Li-Fraumeni Syndrome

People with this syndrome have an increased risk of several types of cancer, including developing stomach cancer at a relatively young age. Li-Fraumeni syndrome is caused by a mutation in the TP53 gene.

Peutz-Jeghers Syndrome (PJS)

People with this condition develop polyps in the stomach and intestines, as well as in other areas including the nose, the airways of the lungs, and the bladder. The polyps in the stomach and intestines are a special type called hamartomas. They can cause problems like bleeding or blockage of the intestines. PJS can also cause dark freckle-like spots on the lips, inner cheeks and other areas. People with PJS have an increased risk of cancers of the breast, colon, pancreas, stomach, and several other organs. This syndrome is caused by mutations in the gene STK1.

Family History of Stomach Cancer

People with first-degree relatives (parents, siblings, or children) who have had stomach cancer are more likely to develop this disease.

Some Types of Stomach Polyps

Polyps are non-cancerous growths on the lining of the stomach. Most types of polyps (such as hyperplastic polyps or inflammatory polyps) do not seem to increase a person's risk of stomach cancer, but adenomatous polyps—also called adenomas—can sometimes develop into cancer.

Epstein-Barr Virus (EBV) Infection

Epstein-Barr virus causes infectious mononucleosis (also called mono). Almost all adults have been infected with this virus at some time in their lives, usually as children or teens.

EBV has been linked to some forms of lymphoma. It is also found in the cancer cells of about 5% to 10% of people with stomach cancer. These people tend to have a slower growing, less aggressive cancer with a lower tendency to spread. EBV has been found in some stomach cancer cells, but it isn't yet clear if this virus actually causes stomach cancer.

Certain Occupations

Workers in the coal, metal, and rubber industries seem to have a higher risk of getting stomach cancer.

Common Variable Immune Deficiency (CVID)

People with CVID have an increased risk of stomach cancer. The immune system of someone with CVID can't make enough antibodies in response to germs. People with CVID have frequent infections as well as other problems, including atrophic gastritis and pernicious anaemia. They are also more likely to get gastric lymphoma and stomach cancer.

### Gastric Cancer Symptoms

The following is adapted from the American Cancer Society, Stomach Cancer Early Detection, Diagnosis, and Staging.

Symptoms of gastric cancer include the following:
Poor appetite
Weight loss (without trying)
Abdominal (belly) pain
Vague discomfort in the abdomen, usually above the navel
A sense of fullness in the upper abdomen after eating a small meal
Heartburn or indigestion
Nausea
Vomiting, with or without blood
Swelling or fluid build-up in the abdomen
Blood in the stool
Low red blood cell count (anaemia)

Early-stage stomach cancer rarely causes symptoms. This is one of the reasons stomach cancer is so hard to detect early.

Since symptoms of stomach cancer often do not appear until the disease is advanced, only about 1 in 5 stomach cancers in the United States is found at an early stage, before it has spread to other areas of the body.

Upper Endoscopy

Upper Endoscopy (also called esophagogastroduodenoscopy or EGD) is the main test used to find stomach cancer. It may be used when someone has certain risk factors or when signs and symptoms suggest this disease may be present.

During this test, the doctor passes an endoscope, which is a thin, flexible, lighted tube with a small video camera on the end, down an individual's throat. This lets the doctor see the lining of the oesophagus, stomach, and first part of the small intestine.

If abnormal areas are seen, biopsies (tissue samples) can be taken using instruments passed through the endoscope. The tissue samples are sent to a lab, where they are looked at with a microscope to see if cancer is present.

When seen through an endoscope, stomach cancer can look like an ulcer, a mushroom-shaped or protruding mass, or diffuse, flat, thickened areas of mucosa known as linitis plastica. Unfortunately, the stomach cancers in hereditary diffuse gastric cancer syndrome often cannot be seen during endoscopy.

Endoscopy can also be used as part of a special imaging test known as endoscopic ultrasound, which is described below.

This test is usually done under sedation.

Endoscopic Ultrasound

In endoscopic ultrasound (EUS), a small transducer is placed on the tip of an endoscope. While the patient is sedated sedated, the endoscope is passed down the throat and into the stomach. This lets the transducer rest directly on the wall of the stomach where the cancer is. The layers of the stomach wall, as well as the nearby lymph nodes and other structures just outside the stomach, may then be examined. The picture quality is better than a standard ultrasound because of the shorter distance the sound waves have to travel.

EUS is most useful in seeing how far a cancer may have spread into the wall of the stomach, to nearby tissues, and to nearby lymph nodes. It may also be used to help guide a needle into a suspicious area to get a tissue sample (EUS-guided needle biopsy).

Biopsy

If an abnormal-looking area is seen on endoscopy or an imaging test, a biopsy may be performed to confirm the diagnosis.

Biopsies to check for stomach cancer are most often obtained during upper endoscopy. If the doctor sees any abnormal areas in the stomach lining during the endoscopy, instruments can be passed down the endoscope to biopsy them. Some stomach cancers are deep within the stomach wall, which may make them hard to biopsy with standard endoscopy. If the doctor suspects cancer might be deeper in the stomach wall, endoscopic ultrasound may be used to guide a thin, hollow needle into the wall of the stomach to get a biopsy sample.

Biopsies may also be taken from areas of possible cancer spread, such as nearby lymph nodes or suspicious areas in other parts of the body.

Testing Biopsy Samples

Biopsy samples are sent to a lab to be looked at under a microscope. The samples are checked to see if they contain cancer, and if they do, what kind it is (for example, adenocarcinoma, carcinoid, gastrointestinal stromal tumor, or lymphoma).

More testing may be done if a sample contains certain types of cancer cells. For instance, the tumor may be tested to see if it has too much of a growth-promoting protein called HER2. Tumors with increased levels of HER2 are called HER2-positive.

Stomach cancers that are HER2-positive may be treated with drugs that target the HER2 protein, such as trastuzumab (Herceptin®).

The biopsy sample may be tested in 2 different ways:

Immunohistochemistry (IHC): In this test, special antibodies that stick to the HER2 protein are applied to the sample, which causes cells to change color if many copies are present. This color change can be seen under a microscope. The test results are reported as 0, 1+, 2+, or 3+.

Fluorescent in situ hybridization (FISH): This test uses fluorescent pieces of DNA that specifically stick to copies of the HER2 gene in cells, which can then be counted under a special microscope.

Often the IHC test is used first.

If the results are 0 or 1+, the cancer is HER2-negative. People with HER2-negative tumors are not treated with drugs (like trastuzumab) that target HER2.

If the test comes back 3+, the cancer is HER2-positive. Patients with HER2-positive tumors may be treated with drugs like trastuzumab.

When the result is 2+, the HER2 status of the tumor is not clear. This often leads to testing the tumor with FISH.

It's also possible that the tumor may be tested to see if it has a certain amount of an immune checkpoint protein called PD-L1. If it does, the tumor may be treated with an immune checkpoint inhibitor such as pembrolizumab (Keytrude®). This type of treatment may be given if other treatments have stopped working.

Imaging Tests

Imaging tests use x-rays, magnetic fields, sound waves, or radioactive substances to create pictures of the inside of the body. Imaging tests may be done for a number of reasons, including:

To help find out if a suspicious area might be cancerous

To learn how far cancer may have spread

To help determine if treatment has been effective

Upper Gastrointestinal (GI) Series

This is an x-ray test to look at the inner lining of the oesophagus, stomach, and first part of the small intestine. This test is used less often than endoscopy to look for stomach cancer or other stomach problems, as it can miss some abnormal areas and does not allow the doctor to take biopsy samples. But it is less invasive than endoscopy, and it might be useful in some situations.

For this test, the patient drinks a white chalky solution containing a substance called barium. The barium coats the lining of the oesophagus, stomach, and small intestine. Several x-ray pictures are then taken. Because x-rays can't pass through the coating of barium, this will outline any abnormalities of the lining of these organs.

A double-contrast technique may be used to look for early stomach cancer. With this technique, after the barium solution is swallowed, a thin tube is passed into the stomach and air is pumped in. This makes the barium coating very thin, so even small abnormalities will show up.

Computed Tomography (CT or CAT) Scan

A CT scan uses x-rays to make detailed, cross-sectional images of the body. Unlike a regular x-ray, a CT scan creates detailed images of the soft tissues in the body.

CT scans show the stomach fairly clearly and often can confirm the location of the cancer. CT scans can also show the organs near the stomach, such as the liver, as well as lymph nodes and distant organs where cancer might have spread. The CT scan can help determine the extent (stage) of the cancer and if surgery may be a good treatment option.

CT-guided needle biopsy: CT scans can also be used to guide a biopsy needle into a suspected area of cancer spread. The patient remains on the CT scanning table while a doctor moves a biopsy needle through the skin toward the mass. CT scans are repeated until the needle is within the mass. A fine-needle biopsy sample (tiny fragment of tissue) or a core-needle biopsy sample (a thin cylinder of tissue) is then removed and looked at under a microscope.

Magnetic Resonance Imaging (MRI) Scan

Like CT scans, MRI scans show detailed images of soft tissues in the body. But MRI scans use radio waves and strong magnets instead of x-rays.

Positron Emission Tomography (PET) Scan

For a PET scan, the patient are injected with a slightly radioactive form of sugar, which collects mainly in cancer cells. A special camera is then used to create a picture of areas of radioactivity in the body. The picture is not detailed like a CT or MRI scan, but a PET scan can look for possible areas of cancer spread in all areas of the body at once.

Some newer machines can do both a PET and CT scan at the same time (PET/CT scan). This lets the doctor see areas that “light up” on the PET scan in more detail.

PET is sometimes useful if a doctor thinks the cancer might have spread but doesn't know where. The picture is not detailed like a CT or MRI scan, but it provides helpful information about the whole body. Although PET scans can be useful for finding areas of cancer spread, they aren't always helpful in certain kinds of stomach cancer because these types don't take up glucose very much.

Chest X-Ray

This test can help find out if the cancer has spread to the lungs. It might also determine if there are any serious lung or heart diseases present. This test is not needed if a CT scan of the chest has been done.

Laparoscopy

If this procedure is done, it is usually only after stomach cancer has already been found. Although CT or MRI scans can make detailed pictures of the inside of the body, they can miss some tumors, especially very small tumors. Doctors might do a laparoscopy before any other surgery to help confirm the cancer is still only in the stomach and can be removed completely with surgery. It may also be done before chemotherapy and/or radiation if these are planned before surgery.

This procedure is done in an operating room with the patient under general anesthesia (in a deep sleep). A laparoscope (a thin, flexible tube) is inserted through a small surgical opening in the patient's side. The laparoscope has a small video camera on its end, which sends pictures of the inside of the abdomen to a TV screen. Doctors can look closely at the surfaces of the organs and nearby lymph nodes, or even take small samples of tissue. If it doesn't look like the cancer has spread, sometimes the doctor will “wash” the abdomen with saline (salt water) this is called peritoneal washing. The fluid is then removed and checked to see if it contains cancer cells. If it does, the cancer has spread, even if the spread couldn't be seen.

Sometimes laparoscopy is combined with ultrasound to give a better picture of the cancer.

Lab Tests

When looking for signs of stomach cancer, a doctor may order a blood test called a complete blood count (CBC) to look for anaemia (which could be caused by the cancer bleeding into the stomach). A faecal occult blood test may be done to look for blood in stool (faeces) that can't be seen by the naked eye.

The doctor might recommend other tests if cancer is found, especially if a patient is going to have surgery. For instance, blood tests will be done to make sure the liver and kidney functions are normal and that blood clots normally. If surgery is planned or a patient is going to get medicines that can affect the heart, he or she may also have an electrocardiogram (EKG) and echocardiogram (an ultrasound of the heart) to make sure their heart is functioning well.

### Treatment of Gastric Cancer

The methods of diagnosing gastric cancer may be accompanied by a treatment for that disease.

We therefore disclose method of treatment of a gastric cancer in an individual. The method may comprise diagnosing gastric cancer by a method as set out in this document. where the individual is determined to be suffering from, or likely to suffer from, gastric cancer, the method may comprise administering to the individual a treatment for gastric cancer.

The treatment of gastric cancer commonly comprise one or more of the following interventions: surgery, radiotherapy, administering a chemotherapeutic agent, administering an immunotherapeutic agent, or the use of targeted therapies such as trastuzumab and ramucirumab.

Potential therapeutic agents to be administered for the treatment of gastric cancer may comprise small molecules, antibodies, vaccines or peptides.

Chemotherapeutic agents for use in the treatment of gastric cancer include 5-fluorouracil, capecitabine, carboplatin, cisplatin, docetaxel, epirubicin, irinotecan, oxaliplatin, paclitaxel, trifluridine and tipiracil.

Immunotherapeutic agents for use in the treatment of gastric cancer includes immune checkpoint inhibitors such as pembrolizumab.

The treatment of choice for early stage gastric cancer is usually surgery. Treatment by endoscopic resection is also possible for cancers identified at an early stage.

It is generally recognised that the treatment outcomes for patients identified with early stage gastric cancer is significantly better than patients with later stage gastric cancer (Lello et al, 2007).

### Detection and Diagnostic Methods

Detection of Expression of hsa-miR-484, hsa-miR-186-5p, hsa-miR-142-5p, hsa-miR-320d, hsa-miR-320a, hsa-miR-320b and hsa-miR-17-5p miRNAs (optionally together with hsa-miR-423-5p)

We show in the Examples that the expression of hsa-miR-484, hsa-miR-186-5p, hsa-miR-142-5p, hsa-miR-320d, hsa-miR-320a, hsa-miR-320b and hsa-miR-17-5p miRNAs, as well as hsa-miR-423-5p, in gastric cancer patients is altered (up-regulated or down-regulated) when compared to normal individuals.

Accordingly, we provide for a method of diagnosis of cancer, including gastric cancer such as metastatic, aggressive or invasive gastric cancer, comprising detecting modulation of expression of hsa-miR-484, hsa-miR-186-5p, hsa-miR-142-5p, hsa-miR-320d, hsa-miR-320a, hsa-miR-320b and hsa-miR-17-5p miRNAs, optionally together with hsa-miR-423-5p, such as up-regulation or down-regulation of expression of hsa-miR-484, hsa-miR-186-5p, hsa-miR-142-5p, hsa-miR-320d, hsa-miR-320a, hsa-miR-320b, hsa-miR-17-5p or hsa-miR-423-5p miRNA in a cell or tissue of an individual.

Such detection may also be used to determine whether a cell will become invasive or aggressive. Thus, detection of a modulated level of hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, miRNA expression, amount or activity in the cell—such as via a sample from an organism comprising the cell—may indicate that the cell is likely to be or become aggressive, metastatic or invasive.

It will be appreciated that as the level of miRNAs hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, varies with the aggressiveness of a tumour, that detection of hsa-miR-484, hsa-miR-186-5p, hsa-miR-142-5p, hsa-miR-320d, hsa-miR-320a, hsa-miR-320b, hsa-miR-17-5p, as well as optionally hsa-miR-423-5p, miRNA expression, amount or activity may also be used to predict a survival rate of an individual with cancer. Detection of expression, amount or activity of hsa-miR-484, hsa-miR-186-5p, hsa-miR-142-5p, hsa-miR-320d, hsa-miR-320a, hsa-miR-320b and hsa-miR-17-5p miRNAs may therefore be used as a method of prognosis of an individual with cancer.

Detection of hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, miRNA expression, amount or level may be used to determine the likelihood of success of a particular therapy in an individual with a cancer. It may be used in a method of determining whether a tumour in an individual is, or is likely to be, an invasive or metastatic tumour.

The diagnostic methods described in this document may be combined with the therapeutic methods described. Thus, we provide for a method of treatment, prophylaxis or alleviation of cancer in an individual, the method comprising detecting modulation of expression, amount or activity of hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, miRNAs in an individual and administering an appropriate therapy to the individual.

The presence and quantity of hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, miRNAs may be detected in a sample as described in further detail below. Thus, gastric cancer can be diagnosed by methods comprising determining from a sample derived from a subject an abnormally decreased or increased expression, amount or activity, such as a increased expression, amount or activity, of the hsa-miR-484, hsa-miR-186-5p, hsa-miR-142-5p, hsa-miR-320d, hsa-miR-320a, hsa-miR-320b, hsa-miR-17-5p and hsa-miR-423-5p miRNA.

The sample may comprise a cell or tissue sample from an organism or individual suffering or suspected to be suffering from a disease associated with increased, reduced or otherwise abnormal hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, miRNA expression, amount or activity, including spatial or temporal changes in level or pattern of expression, amount or activity. The level or pattern of expression, amount or activity of hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, miRNA in an organism suffering from or suspected to be suffering from such a disease may be usefully compared with the level or pattern of expression, amount or activity in a normal organism as a means of diagnosis of disease.

The sample may comprise a cell or tissue sample from an individual suffering or suspected to be suffering from gastric cancer, such as a serum sample.

In some embodiments, an increased level of expression, amount or activity of hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, miRNA is detected in the sample. The level of hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, miRNA may be increased or decreased to a significant extent when compared to normal cells, or cells known not to be cancerous. Such cells may be obtained from the individual being tested, or another individual, such as those matched to the tested individual by age, weight, lifestyle, etc.

Increase in Expression of hsa-miR-484, has-miR-186-5p, hsa-miR-320d, hsa-miR-320a or hsa-miR-320b

In some embodiments, the level of expression, amount or activity of the miRNA is increased by 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200% or more. In some embodiments, the level of expression, amount or activity of the miRNA is increased by 45% or more, such as 50% or more.

For example, gastric cancer may be diagnosed where the expression of one or more of hsa-miR-484, has-miR-186-5p, hsa-miR-320d, hsa-miR-320a or hsa-miR-320b is increased, compared to an individual known not to be suffering from gastric cancer.

Decrease in Expression of hsa-miR-142-5p or hsa-miR-17-5p

In other embodiments, the level of expression, amount or activity of the miRNA is decreased by 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200% or more. In some embodiments, the level of expression, amount or activity of the miRNA is decreased by 45% or more, such as 50% or more.

Gastric cancer may therefore be diagnosed where the expression of one or more of hsa-miR-142-5p and hsa-miR-17-5p is decreased, compared to an individual known not to be suffering from gastric cancer.

### Detection of Expression of Mirnas

The expression, amount or activity of hsa-miR-484, hsa-miR-186-5p, hsa-miR-142-5p, hsa-miR-320d, hsa-miR-320a, hsa-miR-320b and hsa-miR-17-5p miRNA, optionally together with hsa-miR-423-5p miRNA, may be detected in a number of ways, as known in the art, and as described in further detail below. Typically, the amount of hsa-miR-484, hsa-miR-186-5p, hsa-miR-142-5p, hsa-miR-320d, hsa-miR-320a, hsa-miR-320b or hsa-miR-17-5p miRNA, optionally with hsa-miR-423-5p, in a sample of tissue from an individual is measured, and compared with a sample from an unaffected individual.

Detection of the amount, activity or expression of hsa-miR-484, hsa-miR-186-5p, hsa-miR-142-5p, hsa-miR-320d, hsa-miR-320a, hsa-miR-320b and hsa-miR-17-5p miRNAs, optionally together with hsa-miR-423-5p miRNA, may be used to grade gastric cancer. For example, a high level of amount, activity or expression of hsa-miR-484, hsa-miR-186-5p, hsa-miR-142-5p, hsa-miR-320d, hsa-miR-320a, hsa-miR-320b and hsa-miR-17-5p miRNAs, optionally together with hsa-miR-423-5p miRNA, may indicate an aggressive, invasive or metastatic cancer. Similarly, a low level of amount, activity or expression of hsa-miR-484, hsa-miR-186-5p, hsa-miR-142-5p, hsa-miR-320d, hsa-miR-320a, hsa-miR-320b or hsa-miR-17-5p miRNA may indicate a non-aggressive, non-invasive or non-metastatic cancer.

Levels of hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, miRNAs gene expression may be determined using a number of different techniques.

In one embodiment, we disclose a method of detecting the presence of a nucleic acid comprising a hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, miRNA nucleic acid in a sample, by contacting the sample with at least one nucleic acid probe which is specific for the hsa-miR-484, hsa-miR-186-5p, hsa-miR-142-5p, hsa-miR-320d, hsa-miR-320a, hsa-miR-320b or hsa-miR-17-5p, optionally together with hsa-miR-423-5p, miRNA and monitoring the sample for the presence of the miRNA. For example, the nucleic acid probe may specifically bind to the hsa-miR-484, hsa-miR-186-5p, hsa-miR-142-5p, hsa-miR-320d, hsa-miR-320a, hsa-miR-320b or hsa-miR-17-5p, optionally together with hsa-miR-423-5p, miRNA, or a portion of it, and binding between the two detected; the presence of the complex itself may also be detected.

Thus, in one embodiment, the amount of hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, miRNA may be measured in a sample. hsa-miR-484, hsa-miR-186-5p, hsa-miR-142-5p, hsa-miR-320d, hsa-miR-320a, hsa-miR-320b and hsa-miR-17-5p miRNA, and optionally hsa-miR-423-5p, may be assayed by in situ hybridization, Northern blotting or reverse transcriptase-polymerase chain reaction. Nucleic acid sequences may be identified by in situ hybridization, Southern blotting, single strand conformational polymorphism, PCR amplification and DNA-chip analysis using specific primers. (Kawasaki, 1990; Sambrook, 1992; Lichter et al, 1990; Orita et al, 1989; Fodor et al., 1993; Pease et al., 1994).

hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, miRNA RNA may be extracted from cells using RNA extraction techniques including, for example, using acid phenol/guanidine isothiocyanate extraction (RNAzol B; Biogenesis), or RNeasy RNA preparation kits (Qiagen).Typical assay formats utilising ribonucleic acid hybridisation include nuclear run-on assays, RT-PCR and RNase protection assays (Melton et al., Nuc. Acids Res. 12:7035. Methods for detection which can be employed include radioactive labels, enzyme labels, chemiluminescent labels, fluorescent labels and other suitable labels.

Each of these methods allows quantitative determinations to be made, and are well known in the art. Decreased or increased hsa-miR-484, hsa-miR-186-5p, hsa-miR-142-5p, hsa-miR-320d, hsa-miR-320a, hsa-miR-320b or hsa-miR-17-5p, and optionally hsa-miR-423-5p, miRNA expression, amount or activity can therefore be measured at the RNA level using any of the methods well known in the art for the quantitation of polynucleotides. Any suitable probe from a or hsa-miR-17-5p miRNA sequence, for example, any portion of a suitable human hsa-miR-484, hsa-miR-186-5p, hsa-miR-142-5p, hsa-miR-320d, hsa-miR-320a, hsa-miR-320b or hsa-miR-17-5p miRNA sequence may be used as a probe. Sequences for designing hsa-miR-484, hsa-miR-186-5p, hsa-miR-142-5p, hsa-miR-320d, hsa-miR-320a, hsa-miR-320b and hsa-miR-17-5p miRNA probes may be derived from sequences having relevant miRBase accession numbers, or a portion of such sequences.

Typically, RT-PCR is used to amplify RNA targets. In this process, the reverse transcriptase enzyme is used to convert RNA to complementary DNA (cDNA) which can then be amplified to facilitate detection.

Many DNA amplification methods are known, most of which rely on an enzymatic chain reaction (such as a polymerase chain reaction, a ligase chain reaction, or a self-sustained sequence replication) or from the replication of all or part of the vector into which it has been cloned.

Many target and signal amplification methods have been described in the literature, for example, general reviews of these methods in Landegren, U. et al., Science 242:229-237 (1988) and Lewis, R., Genetic Engineering News 10:1, 54-55 (1990).

For example, the polymerase chain reaction may be employed to detect hsa-miR-484, hsa-miR-186-5p, hsa-miR-142-5p, hsa-miR-320d, hsa-miR-320a, hsa-miR-320b and hsa-miR-17-5p miRNA.

The “polymerase chain reaction” or “PCR” is a nucleic acid amplification method described inter alia in U.S. Pat. Nos. 4,683,195 and 4,683,202. PCR can be used to amplify any known nucleic acid in a diagnostic context (Mok et al., 1994, Gynaecologic Oncology 52:247-252). Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al., 1990, Proc. Natl. Acad. Sci. USA 87:1874). Ligation amplification reaction or ligation amplification system uses DNA ligase and four oligonucleotides, two per target strand. This technique is described by Wu, D. Y. and Wallace, R. B., 1989, Genomics 4:560. In the Qβ Replicase technique, RNA replicase for the bacteriophage Qβ, which replicates single-stranded RNA, is used to amplify the target DNA, as described by Lizardi et al., 1988, Bio/Technology 6:1197.

A PCR procedure basically involves: (1) treating extracted DNA to form single-stranded complementary strands; (2) adding a pair of oligonucleotide primers, wherein one primer of the pair is substantially complementary to part of the sequence in the sense strand and the other primer of each pair is substantially complementary to a different part of the same sequence in the complementary antisense strand; (3) annealing the paired primers to the complementary sequence; (4) simultaneously extending the annealed primers from a 3′ terminus of each primer to synthesize an extension product complementary to the strands annealed to each primer wherein said extension products after separation from the complement serve as templates for the synthesis of an extension product for the other primer of each pair; (5) separating said extension products from said templates to produce single-stranded molecules; and (6) amplifying said single-stranded molecules by repeating at least once said annealing, extending and separating steps.

Reverse transcription-polymerase chain reaction (RT-PCR) may be employed. Quantitative RT-PCR may also be used. Such PCR techniques are well known in the art, and may employ any suitable primer from a hsa-miR-484, hsa-miR-186-5p, hsa-miR-142-5p, hsa-miR-320d, hsa-miR-320a, hsa-miR-320b or hsa-miR-17-5p miRNA sequence.

Alternative amplification technology can also be exploited. For example, rolling circle amplification (Lizardi et al., 1998, Nat Genet 19:225) is an amplification technology available commercially (RCAT™) which is driven by DNA polymerase and can replicate circular oligonucleotide probes with either linear or geometric kinetics under isothermal conditions. A further technique, strand displacement amplification (SDA; Walker et al., 1992, Proc. Natl. Acad. Sci. USA 80:392) begins with a specifically defined sequence unique to a specific target.

### Diagnostic Kits

We also provide diagnostic kits for detecting gastric cancer in an individual, or susceptibility to gastric cancer in an individual.

The diagnostic kit may comprise means for detecting expression, amount or activity of hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, miRNA in the individual, by any means as described in this document. The diagnostic kit may therefore comprise any one or more of the following: a hsa-miR-484, hsa-miR-186-5p, hsa-miR-142-5p, hsa-miR-320d, hsa-miR-320a, hsa-miR-320b, hsa-miR-17-5p and optionally hsa-miR-423-5p miRNA polynucleotide or a fragment thereof or a complementary nucleotide sequence to hsa-miR-484, hsa-miR-186-5p, hsa-miR-142-5p, hsa-miR-320d, hsa-miR-320a, hsa-miR-320b and hsa-miR-17-5p miRNA, optionally together with hsa-miR-423-5p miRNA, or a fragment thereof.

The diagnostic kit may comprise instructions for use, or other indicia. The diagnostic kit may further comprise means for treatment or prophylaxis of gastric cancer, such as any of the compositions described in this document, or any means known in the art for treating gastric cancer.

### Further Aspects

Further aspects and embodiments of the invention are now set out in the following numbered Paragraphs; it is to be understood that the invention encompasses these aspects:

Paragraph 1. A method of diagnosing a gastric cancer, in which the method comprises detecting, in an extracellular vesicle (EV) in or of an individual: the expression level of an miRNA selected from the group consisting of: hsa-miR-484, hsa-miR-186-5p, hsa-miR-142-5p, hsa-miR-320d, hsa-miR-320a, hsa-miR-320b and hsa-miR-17-5p; or a variant, homologue, derivative or fragment thereof such as a sequence having at least 75%, 80%, 85%, 90%, 95%, 96%, 97%, 98% or 99% sequence identity thereto; as compared to the expression level of the miRNA in an EV in or of an individual known not to be suffering from gastric cancer; in which an altered expression level, for example an increased or decreased expression level, preferably an increased expression level, of the miRNA indicates that the individual is suffering, or is likely to be suffering, from gastric cancer.

Paragraph 2. A method according to Paragraph 1, in which: (a) hsa-miR-484 comprises a polynucleotide sequence having miRBase Accession Number MIMAT0002174 or a variant, homologue, derivative or fragment thereof such as a sequence having 75%, 80%, 85%, 90%, 95%, 96%, 97%, 98% or 99% sequence identity thereto and comprising hsa-miR-484 activity; (b) hsa-miR-186-5p comprises a polynucleotide sequence having miRbase Accession Number MIMAT0000456 or a variant, homologue, derivative or fragment thereof such as a sequence having 75%, 80%, 85%, 90%, 95%, 96%, 97%, 98% or 99% sequence identity thereto and comprising hsa-miR-484 activity; (c) hsa-miR-142-5p comprises a polynucleotide sequence having miRBase Accession Number MIMAT0000433 or a variant, homologue, derivative or fragment thereof such as a sequence having 75%, 80%, 85%, 90%, 95%, 96%, 97%, 98% or 99% sequence identity thereto and comprising hsa-miR-142-5p activity; (d) hsa-miR-320d comprises a polynucleotide sequence having miRBase Accession Number MIMAT0006764 or a variant, homologue, derivative or fragment thereof such as a sequence having 75%, 80%, 85%, 90%, 95%, 96%, 97%, 98% or 99% sequence identity thereto and comprising hsa-miR-320d activity; (e) hsa-miR-320a comprises a polynucleotide sequence having miRBase Accession Number M10000542 or a variant, homologue, derivative or fragment thereof such as a sequence having 75%, 80%, 85%, 90%, 95%, 96%, 97%, 98% or 99% sequence identity thereto and comprising hsa-miR-320a activity; (f) hsa-miR-320b comprises a polynucleotide sequence having miRBase Accession Number MIMAT0005792 or a variant, homologue, derivative or fragment thereof such as a sequence having 75%, 80%, 85%, 90%, 95%, 96%, 97%, 98% or 99% sequence identity thereto and comprising hsa-miR-320b activity; or (g) hsa-miR-17-5p comprises a polynucleotide sequence having miRBase Accession Number MIMAT0000070 or a variant, homologue, derivative or fragment thereof such as a sequence having 75%, 80%, 85%, 90%, 95%, 96%, 97%, 98% or 99% sequence identity thereto and comprising hsa-miR-17-5p activity.

Paragraph 3. A method according to Paragraph 1 or 2, in which the method comprises detecting the expression level in an extracellular vesicle (EV) of two or more such miRNAs, for example, three miRNAs, four miRNAs, five miRNAs, six miRNAs or seven miRNAs in the group.

Paragraph 4. A method according to Paragraph 1, 2 or 3, in which the method further comprises detecting the expression level in an extracellular vesicle (EV) of hsa-miR-423-5p (miRBase Accession Number MIMAT0004748) or a variant, homologue, derivative or fragment thereof such as a sequence having at least 75%, 80%, 85%, 90%, 95%, 96%, 97%, 98% or 99% sequence identity thereto and comprising hsa-miR-423-5p activity.

Paragraph 5. A method according to any preceding Paragraph, in which the detection comprises polymerase chain reaction, such as real-time polymerase chain reaction (RT-PCR), multiplex polymerase chain reaction (multiplex PCR), Northern Blot, RNAse protection, microarray hybridisation or RNA sequencing.

Paragraph 6. A method according to any preceding Paragraph, in which the extracellular vesicle (EV) in or of the individual is from a sample in or of the individual, such as a bodily fluid sample such as a nasopharyngeal secretion, urine, serum, lymph, saliva, anal and vaginal secretions, perspiration or semen, of the individual.

Paragraph 7. A combination of two or more nucleic acids specified in any of Paragraphs 1, 2 or 4 or probes capable of binding specifically thereto, such as a combination of nucleic acids immobilised on a substrate, preferably in the form of a microarray or as a multiplex polymerase chain reaction (PCR) kit.

Paragraph 8. A combination according to Paragraph 7, comprising probes capable of binding specifically thereto to each of hsa-miR-484 (miRBase Accession Number MIMAT0002174), hsa-miR-186-5p (miRbase Accession Number MIMAT0000456), hsa-miR-142-5p (miRBase Accession Number MIMAT0000433), hsa-miR-320d (miRBase Accession Number MIMAT0006764), hsa-miR-320a (miRBase Accession Number MI0000542), hsa-miR-320b (miRBase Accession Number MIMAT0005792), hsa-miR-17-5p (miRBase Accession Number MIMAT0000070) and hsa-miR-423-5p (miRBase Accession Number MIMAT0004748).

Paragraph 9. An miRNA selected from the group consisting of hsa-miR-484, hsa-miR-186-5p, hsa-miR-142-5p, hsa-miR-320d, hsa-miR-320a, hsa-miR-320b and hsa-miR-17-5p or a variant, homologue, derivative or fragment thereof such as a sequence having at least 75%, 80%, 85%, 90%, 95%, 96%, 97%, 98% or 99% sequence identity thereto for use in a method of detecting or determining the severity of gastric cancer.

Paragraph 10. A pharmaceutical composition comprising two or more miRNAs selected from the group consisting of: hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, or a variant, homologue, derivative or fragment thereof such as a sequence having at least 75%, 80%, 85%, 90%, 95%, 96%, 97%, 98% or 99% sequence identity thereto together with a pharmaceutically acceptable excipient, carrier or diluent.

Paragraph 11. A diagnostic kit for gastric cancer, the kit comprising two or more miRNAs selected from the group consisting of: hsa-miR-484, hsa-miR-186-5p, hsa-miR-142-5p, hsa-miR-320d, hsa-miR-320a, hsa-miR-320b and hsa-miR-17-5p, optionally together with hsa-miR-423-5p, or a variant, homologue, derivative or fragment thereof such as a sequence having at least 75%, 80%, 85%, 90%, 95%, 96%, 97%, 98% or 99% sequence identity thereto together with instructions for use.

Paragraph 12. A method of treatment of a gastric cancer in an individual, the method comprising performing a method according to any of Paragraphs 1 to 6 and, where the individual is determined to be suffering from, or likely to suffer from, gastric cancer, administering to the individual a treatment for gastric cancer.

Paragraph 13. A method of treating gastric cancer in an individual, the method comprising: (a) receiving results of an assay that measures the expression level of an miRNA selected from the group consisting of: hsa-miR-484, hsa-miR-186-5p, hsa-miR-142-5p, hsa-miR-320d, hsa-miR-320a, hsa-miR-320b and hsa-miR-17-5p or a variant, homologue, derivative or fragment thereof such as a sequence having at least 75%, 80%, 85%, 90%, 95%, 96%, 97%, 98% or 99% sequence identity thereto in an extracellular vesicle (EV) of a sample obtained from an individual, in which the results show the expression level of the miRNA in an EV of the sample; (b) if the expression of the miRNA in an EV of the sample is higher than a reference expression level of an miRNA, the reference expression level being the expression level of the miRNA in an EV of an individual known not to be suffering from gastric cancer, thereby providing or predicting an indication of gastric cancer in the individual, administering a treatment for gastric cancer.

Paragraph 14. A method for treating gastric cancer in an individual, comprising: (a) obtaining the results of an analysis of the expression level of an miRNA selected from the group consisting of: hsa-miR-484, hsa-miR-186-5p, hsa-miR-142-5p, hsa-miR-320d, hsa-miR-320a, hsa-miR-320b and hsa-miR-17-5p or a variant, homologue, derivative or fragment thereof such as a sequence having at least 75%, 80%, 85%, 90%, 95%, 96%, 97%, 98% or 99% sequence identity thereto in an extracellular vesicle (EV) of an individual; and (b) administering a treatment for gastric cancer to the individual if the expression level of the miRNA is above a reference expression level, the reference expression level being the expression level of the miRNA in an EV of an individual known not to be suffering from gastric cancer.

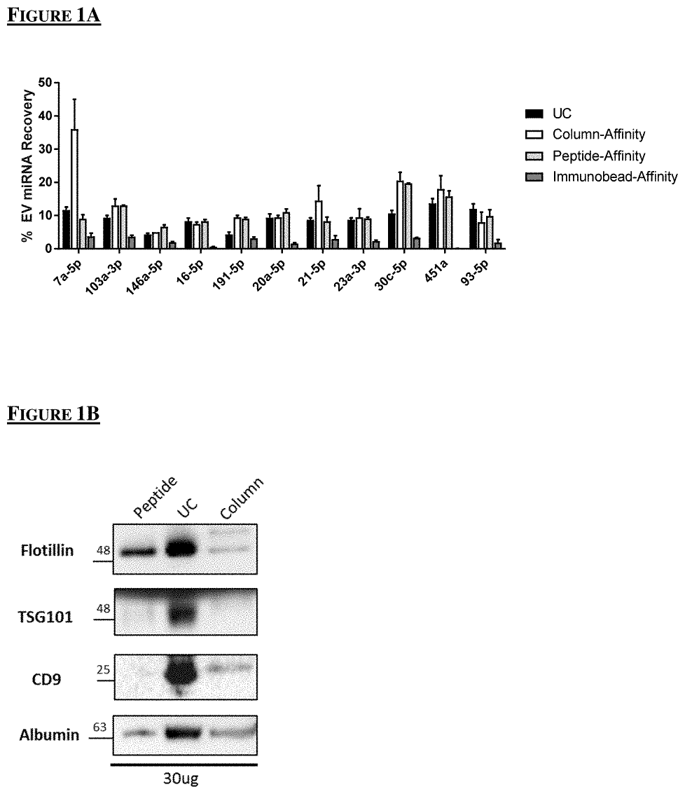

Paragraph 15. A method, combination, miRNA, use, pharmaceutical composition or diagnostic substantially as hereinbefore described with reference to and as shown in FIGS. 1 to 5 of the accompanying drawings.

### EXAMPLES

### Example 1. Materials and Methods—Plasma/Serum Samples

Pooled normal human serum (IPLA-SER) were purchased from Innovative Research, USA. Gastric cancer and healthy control serum samples were purchased from BiolVT, USA.

### Example 2. Materials and Methods—EV Isolation from Serum

Serum was subjected to pre-clearing steps before EV isolation by centrifugation at 2,000 g for 20 minutes (min) followed by 10,000 g for 30 min.

EVs were then isolated from 200 pl pre-cleared serum.

### Example 3. Materials and Methods—Ultracentrifugation

200 μl pre-cleared serum was centrifuged at 100,000 g for 70 min at 4° C. using Optima MAX-XP Ultracentrifuge (Beckman Coulter, USA). Supernatant was aspirated and the pellet was washed and re-centrifuged at 100,000 g for 70 min at 4° C. EV-containing pellet was resuspended in 200 μl phosphate-buffered saline (PBS) for subsequent RNA extraction. For protein analysis, the pellet was dissolved in 30 μl 5% sodium dodecyl sulfate (SDS).

### Example 4. Materials and Methods—Polymer-Based Precipitation

EVs were isolated from 200 μl pre-cleared serum using four commercial polymer-based precipitation reagents: Total Exosome Isolation (from serum) (Invitrogen, SUA), ExoQuick Exosome Precipitation Solution (System Biosciences, USA), miRCURY Exosome Isolation Kit—Serum and Plasma (Exiqon, Denmark) and EXO-prep (HansaBioMed, Estonia) according to manufacturer's protocol. EV pellets were resuspended in 200 μl PBS for subsequent RNA extraction. Pellet was dissolved in 30 μl 5% SDS for protein analysis.

### Example 5. Materials and Methods—Column Affinity-Based Purification

EVs were isolated from 200 μl pre-cleared serum using exoRNeasy Serum/Plasma Midi Kit (Qiagen, Germany) following manufacturer's protocol. Briefly, serum was mixed with binding buffer and loaded onto the membrane column for washing. EVs were then directly lysed by addition of QIAzol and RNA was eluted in 30 μl nuclease-free water.

### Example 6. Materials and Methods—Peptide Affinity-Based Purification

EVs were isolated using ME™ Kit (New England Peptide, USA). 200 μl pre-cleared serum was incubated with 20 μl Vn96 peptide stock overnight at 4° C. with end-to-end rotation. The mixture was centrifuged at 17,000 g at room temperature for 15 min. The supernatant was then removed and EV-containing pellet was washed twice with 500 μl PBS at 17,000 g for 10 min. EV-containing pellet was resuspended in 200 μl PBS for subsequent RNA extraction.

### Example 7. Materials and Methods—Immunoaffinity Affinity-Based Purification

EVs were isolated using ExoCap™ Composite Kit for serum Plasma (JSR Life Sciences, Japan). 100 μl capture beads were mixed with 1 ml treatment buffer and incubated with 200 μl pre-cleared serum for overnight at 4° C. with end-to-end rotation. The supernatant was removed by placing the tube on a magnetic tube stand for one min. Beads were washed twice with 500 μl washing/dilution buffer. Washed beads were resuspended in 200 μl PBS and proceeded to RNA extraction immediately.

### Example 8. Materials and Methods—RNA Isolation from Total Serum or EV Preparations

Total RNA from 200 μl EV preparations or 200 μl neat serum was extracted using miRNeasy serum/plasma miRNA isolation kit (Qiagen) according to manufacturer's protocol.

For normalization of technical variations during RNA isolation, 1 ml of QIAzol lysis buffer was spiked with a set of 3 proprietary synthetic miRNAs (MiRXES, Singapore) before being added to the samples.

Subsequently, 200 μl chloroform was added to the mixture, thoroughly mixed and centrifuged at 18,000 g for 15 min to allow phase separation.

The resulting aqueous phase from each sample was transferred to QiaCube (Qiagen) for automated RNA binding, washing and elution.

RNA was eluted with 30 μl nuclease-free water.

### Example 9. Materials and Methods—Western Blot

EV preparations were lysed with 5% SDS lysis buffer. Protein concentration was determined using DC Protein Assay (Bio-Rad, USA). 30 μg protein was suspended with 4× SDS-PAGE buffer and separated by SDS-polyacrylamide gel electrophoresis (SDS PAGE) at 120 V for 1 hour, followed by transfer to nitrocellulose membrane (0.2 μm) (Bio-Rad) using Trans-Blot® Turbo™ Transfer System (Bio-Rad). The membrane was blocked with 5% milk in PBS +0.1% Tween (PBST) for 30 min at room temperature followed by blotting with primary antibodies against Flotillin (Becton Dickinson, USA), TSG101, CD63, CD81 (Santa Cruz Biotechnology, USA); CD9, Albumin (Abcam, UK) for overnight. The chemiluminescent signal from horseradish peroxidase (HRP)-labeled secondary antibodies (General Electric, USA) was detected using detection reagents according to the manufacturer's instructions (Thermo Scientific, USA).

### Example 10. Materials and Methods—Reverse Transcription (RT)

RNA was reverse transcribed using ID3EAL cDNA synthesis reagents (MiRXES) with modified stem-loop RT primer pool for 11 serum miRNAs (let-7a-5p, miR-103a-3p, miR-146a-5p, miR-16-5p, miR-191-5p, miR-20a-5p, miR-21-5p, miR-23a-3p, miR-30c-5p, miR-451a and miR-93-5p) and 3 exogenous spike-in controls (MiRXES). 5 μl total RNA was mixed with ID3EAL miRNA RT buffer, ID3EAL reverse transcriptase and RT primer pool in a total reaction volume of 15 μl. The reaction mixture was incubated at 42° C. for 30 min followed by 95° C. for 5 min to inactivate the reverse transcriptase on a C1000 Touch™ Thermal Cycler (Bio-Rad).

### Example 11. Materials and Methods—Real-Rime Quantitative PCR (RT-qPCR)

qPCR reaction was carried out using ID3EAL miRNA qPCR reagents (MiRXES) with specific primer pairs for each of the 11 miRNA targets and 3 exogenous spike-in controls.

Each cDNA sample was diluted 10 times with nuclease-free water and added in duplicates into a 384 well plate (Applied Biosystem, USA).

PCR amplification was carried out in a total reaction volume of 15 μl containing 5 μl diluted cDNA, 1× ID3EAL miRNA qPCR master mix, 1× ID3EAL miRNA qPCR primers (MiRXES), topped up with nuclease-free water.

qPCR amplification and detection were performed on QuantStudio 5 Real-Time PCR System (Thermo Scientific) with the following cycling conditions: 95 ° C. for 10 min, 40 ° C. for 5 min, followed by 40 cycles of 95 ° C. for 10 second (sec) and 60 ° C. for 30 sec (optical reading).

Raw cycles to threshold (Ct) values were calculated using QuantStudio Design & Analysis Software v1.5 with automatic baseline setting and a threshold of 0.4.

### Example 12. Materials and Methods—miRNA Profiling

For miRNA profiling, RNA was reverse transcribed using 133 human miRNAs (Supplementary Table 1) grouped in four multiplexes RT primer pools tested to have minimal non-specific interactions between the different RT primers in each group (MiRXES) using ID3EAL cDNA synthesis reagents.

These 133 human miRNAs were selected based on previous profiling studies data and from other literature which showed differential expression in serum between normal and GC samples.

For determination of miRNA copy numbers, six ten-fold serial dilutions of synthetic miRNA template were reverse transcribed with the isolated RNA samples to generate a standard curve from the same microplate.

Using miRNA-specific qPCR assays (MiRXES), 133 candidate miRNAs were measured in each cDNA sample.

Absolute expression copy numbers of each miRNA were determined through interpolation of the Ct values to that of the synthetic miRNA standard curves and adjusted for RT-qPCR efficiency variation.

### Example 13. Materials and Methods—Data Processing

To account for technical variation during RNA isolation, Ct values from samples were normalized using the 3 exogenous spike-in controls: (1) average Ct of the 3 spike-in were calculated per sample (2) average Ct of the 3 spike-in were calculated from all samples (3) ΔCt was calculated (Average_{per sample}−Average_{all sample}) (4) Subtract ΔCt from each Ct values of each miRNA measured in the samples³⁷. Percentage EV miRNA recovery from neat serum was calculated using 2^{-(Ct}ₜₒₜₐₗ^{-Ct}_{EV})×100%. Data are presented as the mean±standard error of mean (SEM) and are representative of at least three independent experiments. Graphs were plotted using GraphPad Prism 8.0 (GraphPad Software, USA).

In the profiling study using discovery set, data were first normalized by exogenous spike-in controls as described above. To perform global normalization, the average Ct value for all miRNAs was used as the normalization factor, on a per sample basis such that each sample will eventually have the same average miRNA expression levels. A set of 5 reference miRNAs was identified using geNorm and NormFinder (Supplementary Table 2) and was used to normalize data derived from the validation set. log2 copy number for each miRNA was used for data analysis using MATLAB vR2019a, (MathWorks, USA). ROC curve (Receiver Operating Characteristics) was computed with true positive rate at y-axis and false positive rate on the x-axis. AUC for selected miRNAs was estimated based on trapezoidal rule using MATLAB.

### Example 14. Results—Different EV Isolation Methods Result in Contrasting miRNA Recovery

Apart from UC and polymer-based precipitation reagent for EV isolation, several other techniques are commercially available in recent years, namely: (1) column affinity-based, (2) peptide affinity-based and (3) immunobead affinity-based EV purification. To determine the recoveries of miRNA using these methods from low volume samples, EVs were isolated from 200 μI of serum and the expression levels of 11 commonly expressed miRNAs in human serum were evaluated by real-time quantitative polymerase chain reaction (RT-qPCR). The recovery of EV miRNA from column or peptide affinity-based method was similar to UC (˜10-20% recovery) (FIG. 1A), except for 7a-5p (˜35% recovery) by column affinity-based method. Of note, minimal amounts of miRNA were recovered using immunobead affinity-based EV purification method. We next determined the expression of common EV markers by western blotting (FIG. 1B). EV isolated using peptide-affinity did not express TSG101 and CD9. Similarly, TSG101 expression was absent in EV purified using column-based method (FIG. 1B). The amount of albumin contamination using these two methods were also lower as compared to UC. Fractions from immunobead affinity-based method was not analyzed by western blotting as the yield was too low for analysis.

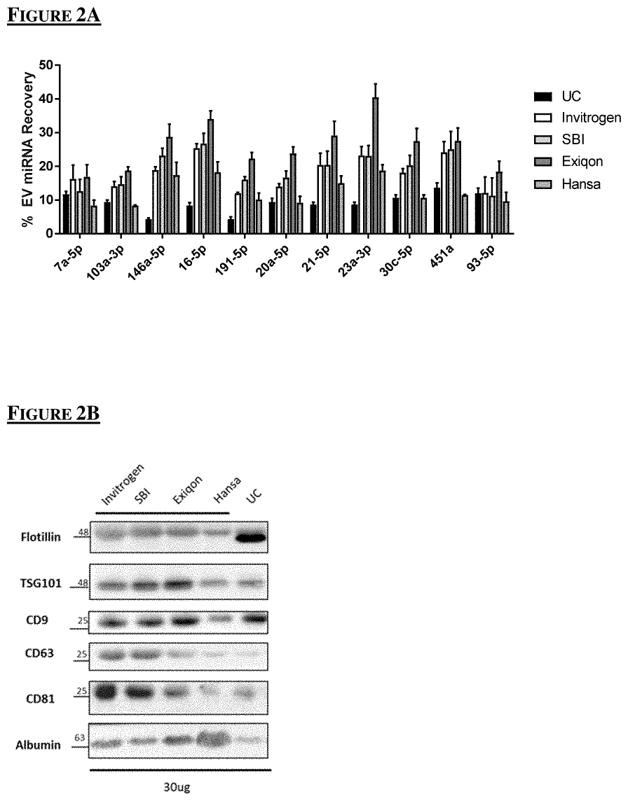

Next, we evaluated EV-associated miRNA recovered using the polymer-based precipitation method. In order to compare the performances of isolating EV from pre-cleared serum, four commercially available precipitation reagents were tested: Invitrogen (lnvt), SBI, Exiqon (Exi) and Hansa (Han). Recovery of miRNAs was higher with almost all reagents tested when compared to UC (FIG. 2A). lnvt and SBI showed similar miRNA recovery, while Exi and Han gave the highest and lowest miRNA yield, respectively. Interestingly, miRNA recovery for 7a-5p and 93-5p were similar to UC across all four reagents.

Western blot analysis revealed the presence of EV markers (Flotillin, TSG101, CD9, CD63, and CD81) in samples isolated using polymer-based precipitation and UC (FIG. 2B). Both lnvt and SBI, which showed similar miRNA recoveries, displayed comparable amounts of EV markers. All four methods displayed variable EV markers expression as compared to UC. With the same amount of protein loaded on western blot, higher albumin contamination was observed with preparations using the Han reagent. These might suggest Han had lesser purified EV after sample preparation. Our results suggested that different polymer-based precipitation methods might be isolating different types of EV.

### Example 15. Results—miRNA Profiling Using Polymer-Based Precipitation Method

We selected polymer-based precipitation method for downstream miRNA profiling analysis as it has several advantages over the other methods. This include the ease of use, relatively low cost, highly scalable, low sample volume requirements, and rapid workflow. We then started testing the hypothesis that quantifying miRNAs in EV fractions may produce greater signal-to-noise results than measuring miRNA in total sera. EVs were isolated from 15 GC sera and 15 controls using lnvt, SBI, Exi or Han precipitation reagents.

Clinical information for all subjects was listed in Table E1 (below).

TABLE E1
Clinical information of serum samples used in the discovery set

| Clinical Information | GC | Normal |
|---|---|---|
| No. of samples | 15 | 15 |
| Age (yr) | 46-79 | 42-56 |
| Gender | | |
| Male | 7 | 10 |
| Female | 8 | 5 |
| Race | | |
| Ukraine | 15 | — |
| Russian | — | 15 |
| Smoking | | |
| No | 15 | 15 |
| Drinking | | |
| No | 15 | 15 |
| AJCC Stage | | |
| IA | 2 | — |
| IB | 4 | — |
| II | 1 | — |
| IIA | 3 | — |
| IIB | 5 | — |
| Metastasis | No | — |

Expression of 133 GC-related miRNAs in both total sera and EV fractions were measured and compared. All miRNAs were detectable in total sera fractions. Out of 133 miRNAs, 30 were not detected in samples isolated with either one of the four polymer-based precipitation methods. As such, these miRNAs were not used for subsequent analysis.

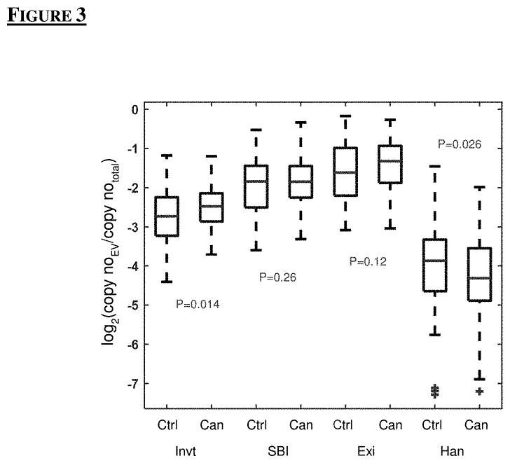

We first examined the differential expression of miRNA in total sera and EV fractions of both cancer and control groups (FIG. 3). A value closer to 0 indicates the level of miRNAs in EV fractions is similar to that in total sera. The Han reagent showed the greatest difference in miRNA expression level in EV fractions as compared to total sera. Han and Invt reagents enabled better discrimination of miRNA levels between cancer and control group than the SBI and Exi reagents (p-value <0.05).

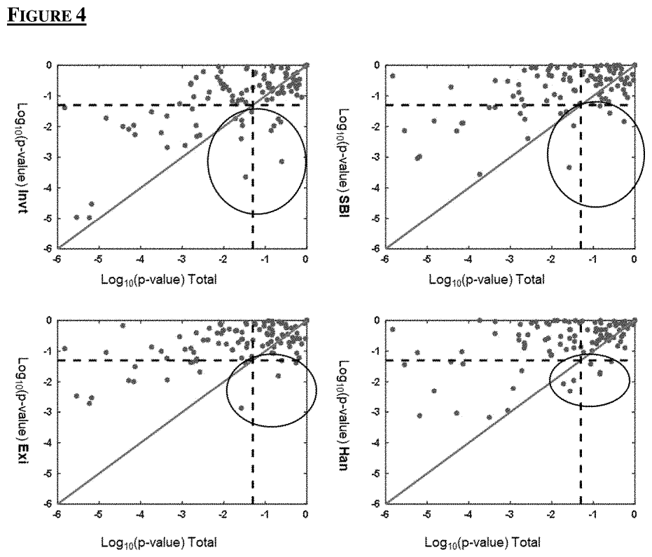

Next, we identified those EV-associated miRNAs with potential diagnostic values by satisfying two criteria: (1) p-value of fold-change between cancer and control in EV fraction is <0.05; (2) p-value in EV fraction is less than total fraction (FIG. 4). 17 miRNAs were identified and their p-values, fold-change and AUC compared to total fraction were listed in Table E2 (below).

TABLE E2
List of EV-associated miRNAs with either p-values, fold-change or AUC better compared to total serum.
P-value <0.05, <0.01 and <0.001 are highlighted in bold, italic and underline respectively.

| | P-value | | | | | Fold-Change [Log₂ (Cancer/Control)] | | | | | AUC | | | | |
|---|---|---|---|---|---|---|---|---|---|---|---|---|---|---|---|
| miRNAs | Total | Invt | SBI | Exi | Han | Total | Invt | SBI | Exi | Han | Total | Invt | SBI | Exi | Han |
| hsa-miR-629-5p | 0.0339 | 0.0002 | 0.0105 | 0.0395 | 0.0109 | 0.42 | 1.01 | 0.7 | 0.59 | 0.9 | 0.75 | 0.89 | 0.81 | 0.77 | 0.93 |
| hsa-miR-423-5p | 0.2505 | 0.0007 | 0.0272 | 0.0488 | 0.3201 | 0.18 | 0.72 | 0.55 | 0.49 | 0.3 | 0.60 | 0.88 | 0.79 | 0.79 | 0.70 |
| hsa-miR-484 | 0.0266 | 0.0040 | 0.0005 | 0.0013 | 0.8918 | 0.38 | 0.48 | 0.65 | 0.56 | 0.04 | 0.77 | 0.82 | 0.93 | 0.92 | 0.50 |
| hsa-miR-186-5p | 0.2078 | 0.0177 | 0.3463 | 0.0154 | 0.2365 | −0.22 | −0.34 | −0.19 | −0.36 | −0.42 | 0.63 | 0.75 | 0.61 | 0.81 | 0.77 |
| hsa-miR-363-3p | 0.0193 | 0.0181 | 0.3385 | 0.4561 | 0.0085 | 0.57 | 0.49 | 0.18 | 0.17 | 1.15 | 0.80 | 0.78 | 0.65 | 0.56 | 0.91 |
| hsa-miR-337-5p | 0.0272 | 0.0159 | 0.3792 | 0.0590 | 0.5011 | −0.8 | −0.67 | −0.35 | −0.71 | 0.46 | 0.74 | 0.75 | 0.61 | 0.75 | 0.70 |
| hsa-miR-27a-3p | 0.0283 | 0.0107 | 0.3189 | 0.2439 | 0.4969 | −0.48 | −0.46 | −0.28 | −0.3 | 0.2 | 0.70 | 0.77 | 0.62 | 0.65 | 0.66 |
| hsa-miR-142-5p | 0.0476 | 0.0300 | 0.0551 | 0.0664 | 0.8278 | −0.43 | −0.43 | −0.5 | −0.42 | 0.09 | 0.65 | 0.74 | 0.73 | 0.71 | 0.68 |
| hsa-miR-320d | 0.1399 | 0.0074 | 0.1095 | 0.2051 | 0.2337 | 0.27 | 0.63 | 0.41 | 0.33 | 0.45 | 0.67 | 0.82 | 0.71 | 0.66 | 0.75 |
| hsa-miR-320a | 0.1635 | 0.0105 | 0.0790 | 0.1347 | 0.2776 | 0.25 | 0.64 | 0.45 | 0.38 | 0.36 | 0.65 | 0.81 | 0.74 | 0.74 | 0.70 |
| hsa-miR-320b | 0.2360 | 0.0141 | 0.0642 | 0.1841 | 0.2210 | 0.2 | 0.56 | 0.44 | 0.34 | 0.46 | 0.64 | 0.78 | 0.77 | 0.71 | 0.75 |
| hsa-miR-17-5p | 0.5546 | 0.3240 | 0.0145 | 0.0410 | 0.6165 | −0.06 | −0.12 | −0.33 | −0.26 | 0.09 | 0.58 | 0.56 | 0.81 | 0.78 | 0.55 |
| hsa-miR-223-3p | 0.1461 | 0.0790 | 0.0467 | 0.0868 | 0.0198 | −0.4 | −0.48 | −0.67 | −0.59 | −0.4 | 0.63 | 0.65 | 0.74 | 0.71 | 0.86 |
| hsa-miR-143-3p | 0.0277 | 0.0748 | 0.3059 | 0.4090 | 0.0048 | 0.8 | 0.55 | 0.45 | 0.4 | 1.02 | 0.74 | 0.70 | 0.66 | 0.54 | 0.89 |
| hsa-miR-140-3p | 0.0870 | 0.1318 | 0.4509 | 0.1453 | 0.0376 | 0.31 | 0.29 | 0.13 | 0.21 | 0.82 | 0.80 | 0.76 | 0.66 | 0.79 | 0.86 |
| hsa-miR-145-5p | 0.1456 | 0.1419 | 0.2846 | 0.3352 | 0.0178 | 0.44 | 0.4 | 0.42 | 0.41 | 0.61 | 0.67 | 0.71 | 0.68 | 0.64 | 0.93 |
| hsa-miR-197-3p | 0.2751 | 0.1242 | 0.1615 | 0.3020 | 0.0456 | 0.28 | 0.49 | 0.5 | 0.42 | 1.12 | 0.65 | 0.69 | 0.72 | 0.68 | 0.86 |

Invt has the most number of miRNAs with greater significant p-values as compared to other polymer-based precipitation methods.

### Example 16. Results—Serum EV Carries a Unique miRNA Signature for GC Diagnosis

Based on the results above, we chose lnvt reagent to validate these EV-associated miRNA biomarkers with another independent set of 20 GC and 20 controls.

Clinical information for all subjects was listed in Table E3 (below).

TABLE E3
Clinical information of serum samples used in the validation set.

| Clinical Information | GC | Normal |
|---|---|---|
| No. of samples | 20 | 20 |
| Age (yr) | 47-64 | 48-58 |
| Gender | | |
| Male | 11 | 13 |
| Female | 9 | 7 |
| Race | | |
| Ukraine | 20 | — |
| Russian | — | 20 |
| Smoking | | |
| No | 20 | 20 |
| Drinking | | |
| No | 20 | 20 |
| AJCC Stage | | |
| IB | 3 | — |
| IIA | 9 | — |
| IIB | 6 | — |
| IIIB | 2 | — |
| metastasis | No | — |

miR-140-3p, miR-145-5p and miR-197-3p were not measured further as they were only found to be enhanced with Han reagent Results were normalized with five miRNAs determined to be stable by both geNorm and NormFinder (Table E4, below).

TABLE E4
List of 5 miRNAs used as normalizer for
EV-associated miRNA biomarker validation.
miRNA normalizers

hsa-miR-30d-5p
hsa-miR-425-5p
hsa-miR-93-5p
hsa-miR-20a-5p
hsa-miR-148b-3p

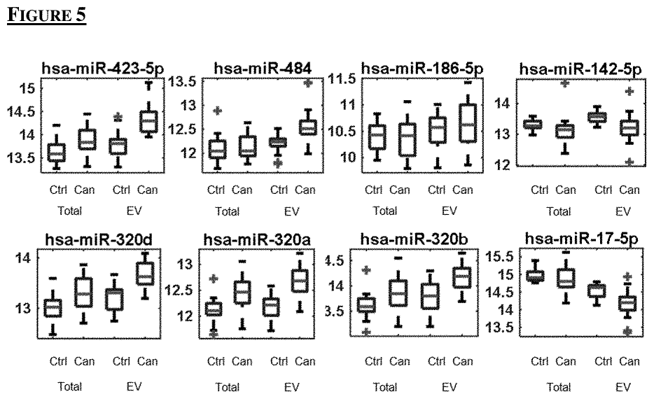

Out of the fourteen miRNAs measured, eight EV-associated miRNAs were found to be differentially expressed in cancer and control samples, consistent with the data in the discovery set (FIG. 5 and Table E5, below).

TABLE E5
List of EV-associated miRNAs measured in the validation set.
miRNAs in bold had p-values, fold-change and AUC better compared
to total serum. P-value <0.05, <0.01 and <0.001 are highlighted
in bold, italic and underline respectively.

| | P-value | | Fold-Change [Log2 (Cancer/Control)] | | AUC | |
|---|---|---|---|---|---|---|
| miRNAs | Total | Invt | Total | Invt | Total | Invt |
| hsa-miR-629-5p | 0.0174 | 0.0075 | 0.32 | 0.43 | 0.73 | 0.73 |
| hsa-miR-423-5p | 0.0015 | 0.0000 | 0.28 | 0.54 | 0.78 | 0.91 |
| hsa-miR-484 | 0.6249 | 0.0001 | 0.04 | 0.39 | 0.56 | 0.90 |
| hsa-miR-186-5p | 0.7323 | 0.2655 | −0.04 | 0.15 | 0.54 | 0.62 |
| hsa-miR-363-3p | 0.0155 | 0.3299 | 0.47 | 0.19 | 0.7 | 0.59 |
| hsa-miR-337-5p | 0.0347 | 0.3083 | −0.56 | −0.28 | 0.68 | 0.54 |
| hsa-miR-27a-3p | 0.0623 | 0.1703 | 0.2 | 0.13 | 0.66 | 0.62 |
| hsa-miR-142-5p | 0.1102 | 0.0029 | −0.18 | −0.35 | 0.75 | 0.82 |
| hsa-miR-320d | 0.0054 | 0.0000 | 0.28 | 0.48 | 0.74 | 0.90 |
| hsa-miR-320a | 0.0017 | 0.0000 | 0.32 | 0.49 | 0.77 | 0.88 |
| hsa-miR-320b | 0.0220 | 0.0001 | 0.23 | 0.4 | 0.72 | 0.83 |
| hsa-miR-17-5p | 0.2760 | 0.0008 | −0.1 | −0.37 | 0.64 | 0.79 |
| hsa-miR-223-3p | 0.2834 | 0.9647 | 0.17 | −0.01 | 0.59 | 0.51 |
| hsa-miR-143-3p | 0.0000 | 0.00084 | 2.02 | 1.32 | 0.91 | 0.76 |

### Example 17. Discussion

Circulating EV released by cancer cells have been widely reported to play a role in cancer biology by communicating with the tumor microenvironment, promoting cell growth and inhibiting the immune system[1, 13, 23, 31, 32, 33]. To facilitate the discovery of EV miRNAs as biomarkers, there is a need to isolate these vesicles promptly and to detect them readily in biofluids. Several EV isolation methods have been developed as an alternative to UC, which is tedious and relies heavily on specialized equipment. These methods include column affinity [34], peptide affinity [35], immunobead affinity to specific antigens (e.g. CD9, CD63, CD81 or EpCAM)[36, 37] and polymer-based precipitation[24, 38, 39, 40], where they are increasingly employed in recent years mainly because of the low cost, high-throughput and low sample volume (as less as 100 μI) requirements. These methods have successfully been used to identify potential EV miRNA biomarkers [4, 41, 42, 43, 44, 45, 46]. To date, there is a lack of a comprehensive evaluation of EV miRNAs across a wide range of methods, a standardized process and comparison of expression levels between diseased and control[47]. In this study, we evaluated a number of commercially available EV isolation methods described above with the intention to develop a rapid, robust process for detecting miRNA with an enhanced signal in EV fractions using RT-qPCR for future clinical use.

Firstly, RNAs were extracted from EV fractions and the recoveries of 11 commonly expressed human miRNAs quantified. Our results showed that EV miRNA recovery from column or peptide affinity-based method was similar to UC, except for let 7a-5p (FIG. 1A). Interestingly, these methods produced EV with differing protein marker profiles, which may indicate different types or collections of multiple types of EV in the fractions (FIG. 1B). TSG101 expression has been reported to be present when using the column-affinity method[34], but absent in our study. The reason for this difference is unclear.

We were not able to quantify miRNAs efficiently using the immunobead affinity-based method. It is possible that the isolated EVs did not contain the miRNAs analyzed in this study or that there were insufficient quantities of EVs isolated. Beads used for capturing EV are coupled with antibodies that recognize EV surface antigens such as CD9, CD63 and CD81. Since these antigens can be readily detected from EV isolated using other methods (FIG. 1B and FIG. 2B), it is likely that the immunobeads were not efficient in pulling down EVs from 200 μI serum in our protocol. However, this method has been shown to isolate EVs from other biological fluids like cell culture supernatants and plasma samples and requires much more input amounts for RT-qPCR analysis [37, 48]. We repeated our study with larger volumes of serum (up to 1000 μl) but the quantities of miRNA detected were close to or below detection limits of the assays (data not shown).

Currently, there are many polymer-based precipitation reagents in the market and we questioned whether these reagents have similar performance. We compared four precipitation reagents (lnvt, SBI, Exi and Han) and found that EV miRNA recovery and profiles do vary among kits (FIG. 2A). This discrepancy may be attributed to the differences in polymers and buffer compositions used. All four reagents tested have higher miRNA recovery when compared to UC. The purity of the sample preparation from polymer-based precipitation was however not as good as UC as evident by the presence of higher amounts of albumin contamination (FIG. 2B). Western blot analysis also demonstrated the EV surface marker profiles for all four precipitation reagents to be different from UC, suggesting that these may have isolated different EV subtypes from UC.

We have identified several EV isolation methods that can be used with low serum volume whereby miRNA recovery is comparable or greater than UC. Currently, there is no general agreement as to the best way to isolate EV from sera. Our intention was to establish a method that is suitable in clinical settings with minimal turnaround time and be easily incorporated into high throughput context and to improve the signal-to-noise ratio of circulating miRNAs in GC patients. Based on these criteria, polymer-based precipitation was the method of choice for the isolation of EV-associated miRNAs and tested the hypothesis that this fraction can improve the detection of miRNAs between cancer and control samples. In the discovery set, 15 GC and 15 control sera were isolated using lnvt, SBI, Exi and Han reagents. miRNA expression levels in EV-associated fractions were compared to miRNA isolated from total sera. Out of 133 miRNAs profiled, 30 were not detectable in some of the samples isolated using lnvt, SBI, Exi or Han reagents. We found that miRNA copy numbers for Han kit prepared samples were much lower than the rest and many miRNAs were undetectable in these samples. We also observed that precipitated pellets from Han treated samples were exceptionally small as compared to lnvt, SBI and Exi. We speculate that the low EV miRNA recovery from Han may be due to the reagent not being efficient in isolating EV from sera, or that Han may be useful for isolating a subset of EV resulting in low concentration of the miRNAs detected in this study. Thus, Han reagent showed a distinct set of miRNA biomarkers (miR-140-3p, miR-145-5p and miR-197-3p) in EV compared to the other three reagents. Nevertheless, 17 EV-associated miRNAs were found to have more significant fold-change, p-value and AUC as compared to total sera with all the four precipitation methods.

We next validated these 17 EV-associated miRNAs using Invt reagent with an independent set of 20 GC and 20 control sera. Our data unveiled a distinct set of eight EV-associated miRNAs that showed superior improvement in diagnostic signal-to-noise ratio as compared to total circulating miRNAs (Table E2). Intriguingly, miR-423-5p, miR-484, miR-142-5p and miR-17-5p are dysregulated[12, 42, 43, 49] and involved in tumourigenesis/metastasis in gastric cancer[41, 44].

In summary, our results demonstrated that differential miRNA expression between cancer and healthy control samples can be readily quantified, both in total and EV-associated fractions. A panel of eight EV-associated miRNAs significantly improved the sensitivity and specificity of current circulating miRNAs for GC diagnosis. Further studies are needed to decipher the origin, specific roles and functions of these eight miRNAs in GC tumourigenesis.

### Example 18. Results: Polymer-based Precipitation Yielded the Highest EV-miRNA Recovery

To identify the best commercially available EV isolation method that is suitable for isolating EV-miRNAs from total serum, we evaluated the EV-miRNA recovery performance of 4 different methods: polymer-based precipitation (PBP), column affinity-based purification (CAP), peptide affinity-based purification (PAP) and immunobead affinity-based purification (IAP). We used a low sample volume (200 μl) to simulate clinical settings. We measured the quantities of 11 commonly-expressed miRNAs in total serum and EVs using RT-qPCR to determine EV-miRNA recovery from each method.

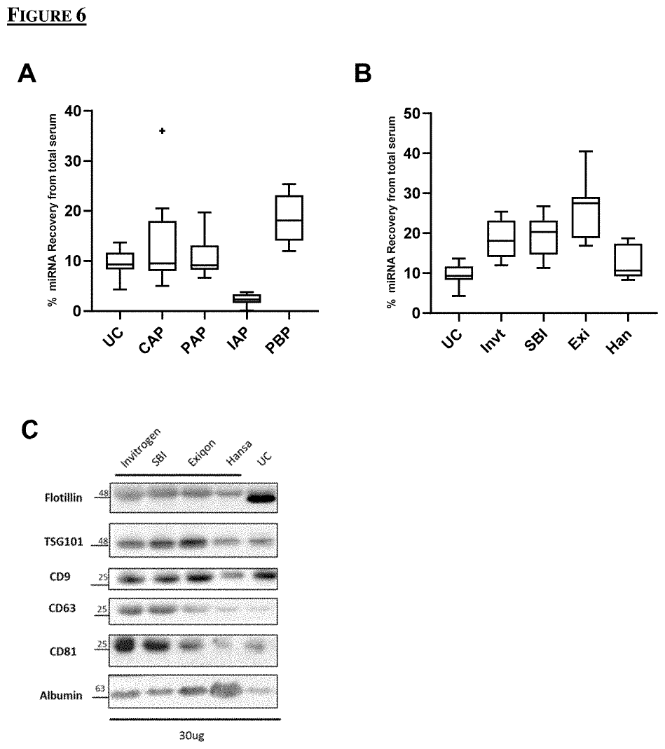

As a control, UC recovered 4-15% of total serum miRNA with an average recovery of 10% (FIG. 6A). Compared to this benchmark, CAP and PAP displayed comparable average miRNA recovered while IAP recovered minimal amounts of miRNA (average recovery below 5%). In contrast, PBP recovered significantly more miRNAs as compared to UC. Since there are several PBP reagents available commercially, we tested these reagents to determine if their performances were comparable. We evaluated 4 PBP reagents from different manufacturers (Invitrogen-Invt, System Biosciences-SBI, Exiqon-Exi, and HansaBioMed -Han) and found that all 4 reagents have higher total serum miRNA recoveries (average recoveries of 20-30%) when compared to UC (FIG. 6B). lnvt and SBI showed similar miRNA recoveries, while Exi and Han gave the highest and lowest miRNA yield, respectively.

We next assayed for the presence of several common EV markers Flotillin, TSG101, CD9, CD63, and CD81 in PBP-isolated samples using Western blotting and were able to detect these markers in all samples (FIG. 6C). This confirmed that each of the PBP reagents had isolated EV from total serum. However, the amount of each EV marker present in EV fractions varied significantly among reagents, suggesting that different reagents may be isolating different amount of EV or EV subtypes. Of note, Han gave the lowest amount of EV marker expression, consistent with it having the lowest EV-miRNA recovery among the PBP protocols tested (FIG. 6B, FIG. 6C). We also observed that PBP-isolated EVs had more albumin contamination compared to UC, with Invt and SBI having the lowest albumin (FIG. 6C).

We, therefore, identified PBP as the preferred EV isolation method with the highest EV-miRNA recovery from total serum. We further showed that all 4 commercially available PBP reagents tested had higher EV-miRNA recovery performance compared to UC, which is the current gold standard for EV isolation. Apart from high EV-miRNA recovery, we also chose PBP for subsequent EV-miRNA biomarker discovery because it was most suited in clinical settings with its ease of use, relatively low-cost, high scalability, low sample volume requirement, and a rapid workflow.

### Example 19. Results: Identification of EV-miRNA Candidates for GC Detection

We next used the 4 commercially available PBP reagents to isolate and identify serum EV-miRNA biomarkers in samples collected from 15 GC and 15 matched healthy controls (clinical information shown in Table NS3 below).

| Clinical Information | GC | Normal |
|---|---|---|
| No. of samples | 15 | 15 |
| Age (yr) | 46-79 | 42-56 |
| Sex | | |
| Male | 7 | 10 |
| Female | 8 | 5 |
| Race | | |
| Ukraine | 15 | — |
| Russian | — | 15 |
| Smoking | | |
| No | 15 | 15 |
| Drinking | | |
| No | 15 | 15 |
| AJCC Stage | | |
| IA | 2 | — |
| IB | 4 | — |
| II | 1 | — |
| IIA | 3 | — |
| IIB | 5 | — |
| Metastasis | No | — |

Table NS3. Clinical information of serum samples used in the discovery set.

To achieve this, we measured the quantities of 133 GC-related miRNAs in total serum and EV fractions isolated using the 4 PBP reagents. All 133 GC-related miRNAs were detected in total serum from all 30 subjects. However, only 104 of these miRNAs were consistently detectable in all EV fractions isolated using the 4 PBP reagents (Table NS4 below). We, therefore, focused on these 104 miRNAs in our subsequent analyses.

TABLE NS4
List of 104 miRNAs which were detectable in EV fractions from all four PBP reagents.
miRNA Name

| | | | |
|---|---|---|---|
| hsa-miR-205-5p | hsa-miR-195-5p | hsa-miR-191-5p | hsa-miR-193b-3p |
| hsa-miR-25-3p | hsa-miR-19a-3p | hsa-miR-194-5p | hsa-miR-21-3p |
| hsa-miR-29b-3p | hsa-miR-197-3p | hsa-miR-29a-3p | hsa-miR-22-3p |
| hsa-miR-29c-5p | hsa-miR-20b-5p | hsa-miR-30e-5p | hsa-miR-30a-5p |
| hsa-miR-30b-5p | hsa-miR-222-3p | hsa-miR-320a | hsa-miR-339-5p |
| hsa-miR-30d-5p | hsa-miR-223-3p | hsa-miR-425-3p | hsa-miR-375 |
| hsa-miR-320d | hsa-miR-23a-5p | hsa-miR-454-3p | hsa-miR-409-3p |
| hsa-miR-328-3p | hsa-miR-29c-3p | hsa-miR-500a-3p | hsa-miR-451a |
| hsa-miR-101-3p | hsa-miR-320b | hsa-miR-629-5p | hsa-miR-485-3p |
| hsa-miR-363-3p | hsa-miR-337-5p | hsa-miR-128-3p | hsa-miR-495-3p |
| hsa-miR-421 | hsa-miR-34a-5p | hsa-miR-885-5p | hsa-miR-550a-5p |
| hsa-miR-425-5p | hsa-miR-362-5p | hsa-miR-92a-3p | hsa-miR-589-5p |
| hsa-miR-501-5p | hsa-miR-106b-3p | hsa-miR-93-5p | hsa-miR-93-3p |
| hsa-miR-629-3p | hsa-miR-378a-3p | hsa-miR-99b-5p | hsa-miR-99a-5p |
| hsa-miR-145-5p | hsa-miR-382-5p | hsa-miR-423-5p | hsa-miR-126-3p |
| hsa-miR-155-5p | hsa-miR-484 | hsa-miR-532-5p | hsa-miR-26a-5p |
| hsa-miR-10b-5p | hsa-miR-487b-3p | hsa-miR-21-5p | hsa-miR-27a-3p |
| hsa-miR-181a-5p | hsa-miR-497-5p | hsa-miR-16-5p | hsa-miR-103a-3p |
| hsa-miR-200c-3p | hsa-miR-106b-5p | hsa-miR-140-5p | hsa-miR-221-3p |
| hsa-miR-486-5p | hsa-miR-671-3p | hsa-miR-148a-3p | hsa-miR-23b-3p |
| hsa-miR-122-5p | hsa-miR-340-5p | hsa-miR-15b-5p | hsa-miR-20a-5p |
| hsa-miR-17-5p | hsa-miR-424-5p | hsa-miR-183-5p | hsa-miR-136-5p |
| hsa-miR-125b-5p | hsa-miR-107 | hsa-miR-186-5p | hsa-miR-139-5p |
| hsa-miR-1280 | hsa-miR-10a-5p | hsa-miR-192-5p | hsa-miR-142-5p |
| hsa-miR-1299 | hsa-miR-140-3p | hsa-miR-19b-3p | hsa-miR-146a-5p |
| hsa-miR-152-3p | hsa-miR-143-3p | hsa-miR-15b-3p | hsa-miR-148b-3p |

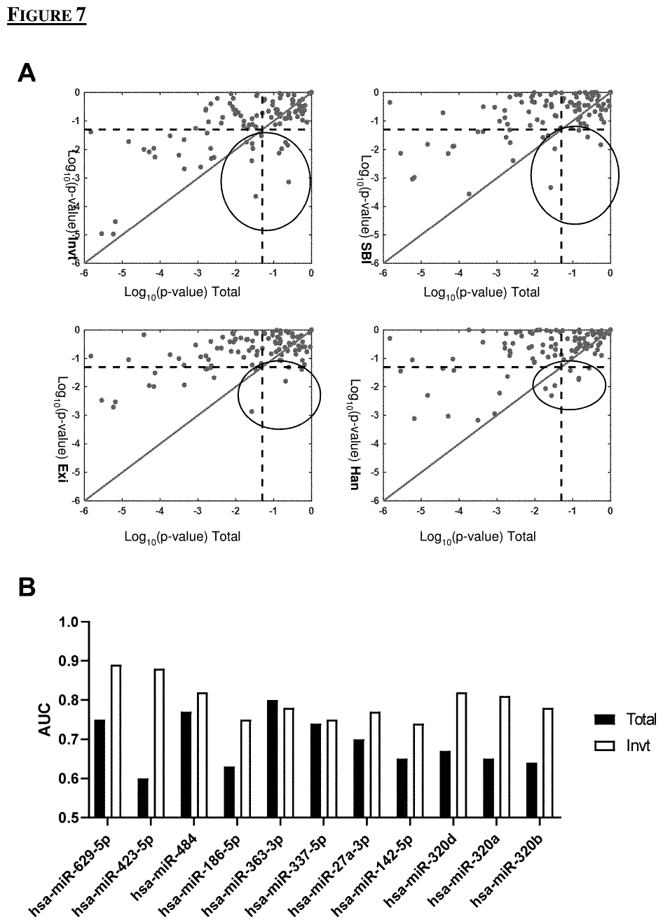

For each PBP method, we looked for potential EV-miRNA biomarkers with enhanced signal-to-noise ratios (differential expression between GC and healthy subjects) over total serum miRNA biomarkers using the following criteria: (1) there is significant EV-miRNA differential expression between GC and healthy samples (potential biomarker), with Student's t-test p-value <0.05; (2) EV-miRNA has lower p-value than p-value for the same miRNA in total serum (enhanced signal-to-noise ratio) (FIG. 7A). Using these criteria, we identified 11, 5, 5, and 7 EV-miRNA biomarker candidates isolated using Invt, SBI, Exi, and Han, respectively (Table N1 below).

TABLE N1
EV-miRNA biomarker candidates identified using 4 PBP protocols.

| Invt* | SBI^{†} | Exi^{‡} | Han^{§} |
|---|---|---|---|
| hsa-miR-629-5p | hsa-miR-629-5p | hsa-miR-629-5p | hsa-miR-629-5p |
| hsa-miR-423-5p | hsa-miR-423-5p | hsa-miR-423-5p | hsa-miR-363-3p |
| hsa-miR-484 | hsa-miR-484 | hsa-miR-484 | hsa-miR-223-3p |
| hsa-miR-186-5p | hsa-miR-17-5p | hsa-miR-186-5p | hsa-miR-143-3p |
| hsa-miR-363-3p | hsa-miR-223-3p | hsa-miR-17-5p | hsa-miR-140-3p |
| hsa-miR-337-5p | | | hsa-miR-145-5p |
| hsa-miR-27a-3p | | | hsa-miR-197-3p |
| hsa-miR-142-5p | | | |
| hsa-miR-320d | | | |
| hsa-miR-320a | | | |
| hsa-miR-320b | | | |

*Total Exosome Isolation (from serum) (Invitrogen)
^{†}ExoQuick Exosome Precipitation Solution (SBI)
^{‡}miRCURY Exosome Isolation Kit - Serum and Plasma (Exiqon)
^{§}EXO-prep (Hansabiomed)

Out of 11 EV-miRNA biomarker candidates isolated by Invt, 10 had higher GC detection accuracy (AUC of ROC curve) as compared to serum miRNA (FIG. 7B and Table N2 below).

TABLE N2
EV-miRNA biomarker candidates identified using Invt. Fold-
change and AUC were listed and compared with total serum.

| | Fold-Change [Log₂ | | | |
|---|---|---|---|---|
| | (Cancer-Contro)] | | AUC (95% CI) | |
| | Total | Invt | Total | Invt |
| hsa-miR-629-5p | 0.42 | 1.01 | 0.75 (0.50-0.91) | 0.89 (0.62-0.99) |
| hsa-miR-423-5p | 0.18 | 0.72 | 0.60 (0.36-0.82) | 0.88 (0.65-0.97) |
| hsa-miR-484 | 0.38 | 0.48 | 0.77 (0.54-0.93) | 0.82 (0.57-0.95) |
| hsa-miR-186-5p | −0.22 | −0.34 | 0.63 (0.40-0.83) | 0.75 (0.52-0.90) |
| hsa-miR-363-3p | 0.57 | 0.49 | 0.80 (0.56-0.95) | 0.78 (0.51-0.93) |
| hsa-miR-337-5p | −0.80 | −0.67 | 0.74 (0.47-0.89) | 0.75 (0.54-0.91) |
| hsa-miR-27a-3p | −0.48 | −0.46 | 0.70 (0.43-0.88) | 0.77 (0.54-0.91) |
| hsa-miR-142-5p | −0.43 | −0.43 | 0.65 (0.38-0.86) | 0.74 (0.49-0.89) |
| hsa-miR-320d | 0.27 | 0.63 | 0.67 (0.41-0.85) | 0.82 (0.57-0.95) |
| hsa-miR-320a | 0.25 | 0.64 | 0.65 (0.42-0.85) | 0.81 (0.56-0.94) |
| hsa-miR-320b | 0.20 | 0.56 | 0.64 (0.38-0.82) | 0.78 (0.54-0.93) |

Since Invt isolated the most EV-miRNA biomarker candidates, we used this reagent for our validation study.

Example 20. Results: Serum EV Carries a Unique miRNA Signature for GC Diagnosis

We used Invt PBP protocol to isolate EV-miRNAs from another independent set of 20 GC and 20 healthy controls (clinical information shown in Table NS5 below).

TABLE NS5
Clinical information of serum samples used in the validation set.

| Clinical Information | GC | Normal |
|---|---|---|
| No. of samples | 20 | 20 |
| Age (yr) | 47-64 | 48-58 |
| Sex | | |
| Male | 11 | 13 |
| Female | 9 | 7 |
| Race | | |
| Ukraine | 20 | — |
| Russian | — | 20 |
| Smoking | | |
| No | 20 | 20 |
| Drinking | | |
| No | 20 | 20 |
| AJCC Stage | | |
| IB | 3 | — |
| IIA | 9 | — |
| IIB | 6 | — |
| IIIB | 2 | — |
| Metastasis | No | — |

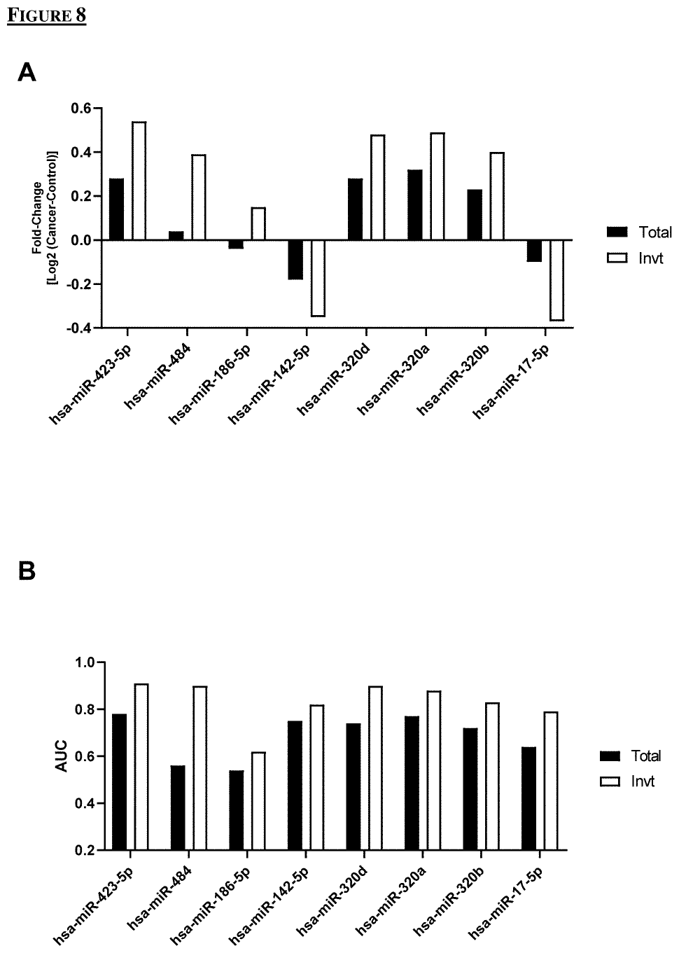

Expression levels of all 11 EV-miRNA biomarker candidates (Table N1) were quantified in total serum and EV fractions. The same criteria (p-value<0.05 and EV p-value<total serum p-value) were used to identify EV-miRNA biomarkers. We validated 8 EV-miRNAs for which EV isolation resulted in enhanced signal-to-noise ratios compared to serum miRNA (FIG. 8A and Table N3 below).

TABLE N3
List of EV-associated miRNAs measured in the validation set. Fold-
change and AUC were listed and compared with total serum.

| | Fold-Change [Log₂ | | | |
|---|---|---|---|---|
| | (Cancer-Control)] | | AUC (95% CI) | |
| | Total | Invt | Total | Invt |
| hsa-miR-423-5p | 0.28 | 0.54 | 0.78 (0.58-0.89) | 0.91 (0.77-0.97) |
| hsa-miR-484 | 0.04 | 0.39 | 0.56 (0.37-0.74) | 0.90 (0.72-0.97) |
| hsa-miR-186-5p | −0.04 | 0.15 | 0.54 (0.34-0.71) | 0.62 (0.42-0.80) |
| hsa-miR-142-5p | −0.18 | −0.35 | 0.75 (0.55-0.88) | 0.82 (0.64-0.93) |
| hsa-miR-320d | 0.28 | 0.48 | 0.74 (0.51-0.88) | 0.90 (0.77-0.97) |
| hsa-miR-320a | 0.32 | 0.49 | 0.77 (0.57-0.91) | 0.88 (0.73-0.97) |
| hsa-miR-320b | 0.23 | 0.40 | 0.72 (0.51-0.87) | 0.83 (0.68-0.94) |
| hsa-miR-17-5p | −0.10 | −0.37 | 0.64 (0.45-0.81) | 0.79 (0.58-0.91) |

All of them had higher AUC values for GC detection compared to serum miRNAs (FIG. 8B and Table N3). The 8 validated PBP-isolated EV-miRNA biomarkers, therefore, had better GC detection accuracy when measured in EV fractions than in total serum, with AUCs ranging from 0.62 to 0.91.

Example 21. Results: Multivariate miRNA Panels for Detection of Gastric Cancer

Further to this, the AUC values for GC detection for multivariate panels comprising 2-, 3-, 4-, 5-, 6-, 7- or 8-miRNAs were evaluated.

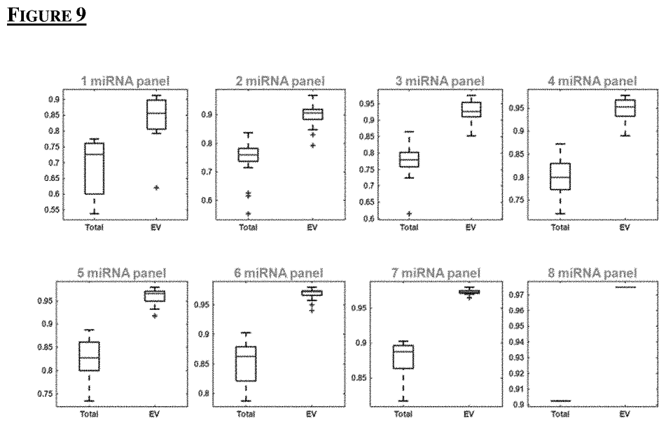

Table N4 lists the median AUC values for these multivariate panels (also represented graphically in FIG. 9) as well as the range of highest to lowest AUC values obtained for the possible combinations of miRNAs for each panel size.

For the 8-miRNA panel, the value provided is the AUC value (there being only one possible combination of the eight miRNAs.

TABLE N4
List of AUC values for multivariate miRNA panels for
the detection of gastric cancer in total serum (Total)
or in extracellular vesicles isolated using Invt.

Median AUC (range)

| | Total | EV |
|---|---|---|
| 2-miRNA | 0.76 (0.55-0.84) | 0.91 (0.79-0.97) |
| 3-miRNA | 0.78 (0.62-0.87) | 0.93 (0.85-0.98) |
| 4-miRNA | 0.80 (0.72-0.87) | 0.95 (0.89-0.98) |
| 5-miRNA | 0.83 (0.74-0.89) | 0.97 (0.92-0.98) |
| 6-miRNA | 0.86 (0.79-0.90) | 0.97 (0.94-0.98) |
| 7-miRNA | 0.89 (0.82-0.90) | 0.97 (0.97-0.98) |
| 8-miRNA | 0.90 | 0.98 |

### Example 22. Discussion

To discover EV-miRNAs as biomarkers for cancer detection, it is essential to isolate EVs rapidly and readily detect them in biofluids. Several EV isolation methods have been developed as alternatives to UC, which is tedious and relies heavily on specialized equipment. These EV isolation methods include CAP³⁸, PAP³⁹, and IAP⁴⁰, ⁴¹, which have been used to isolate EVs with specific antigens (e.g. CD9, CD63, CD81 or EpCAM)⁴⁰, ⁴². Another EV isolation method, PBP, has been increasingly employed in recent years mainly because of its low cost, high-throughput capability, and low sample volume requirements (as little as 100 μl sample)⁴³⁻⁴⁵. Each of these EV isolation methods described above has been successfully used to identify potential EV-miRNA biomarkers⁴⁶⁻⁴⁹. However, to date, there has been no systematic and comprehensive evaluation of EV-miRNA isolation methods and there is no standardized protocol for EV-miRNA isolation. In this study, we evaluated a number of commercially available EV isolation methods with the aim of developing a rapid and robust process for detecting EV-miRNA biomarkers for clinical detection of GC. Specifically, we sought to identify an EV isolation method with high EV-miRNA recovery and test the hypothesis that EV-miRNA isolation using this method would improve the signal-to-noise ratio and GC detection performance of circulating miRNA biomarkers.

We first determined EV-miRNA recovery performance by comparing the quantities of 11 commonly expressed human miRNAs present in total serum and in the EV fraction isolated from serum. We showed that EV-miRNA recoveries using CAP and PAP were comparable to UC while PBP had superior recovery performance. Unexpectedly, we observed very low quantities of EV-miRNAs using IAP. This may indicate that the IAP-isolated EVs did not contain the miRNAs analyzed in this study or that there were insufficient quantities of EVs being isolated, possibly due to the small volume (200 μI) of serum used in our protocol. Although IAP has previously been shown to efficiently isolate EVs from other biological fluids such as cell culture supernatants and plasma samples, these studies used much higher input volumes for RT-qPCR analysis⁴⁰, ⁴². To rule out the possibility that IAP was inefficient in isolating EVs because of the small volume input, we repeated the IAP study with larger volume of serum (up to 1000 μl) and confirmed that EV recovery remained minimal (data not shown). We thus focused on PBP since it had the best EV-miRNA recovery of the methods tested.

Currently, there are many commercially available PBP reagents and we selected 4 of these reagents (lnvt, SBI, Exi and Han) for evaluation in this study. We found different PBP reagents resulted in different EV-miRNA recoveries from total serum. The difference in recovery can be attributed to the differences in polymers and buffer compositions used. Nevertheless, all 4 PBP protocols tested had higher EV-miRNA recoveries compared to UC. However, the Exi and Han PBP protocols had lower EV purity compared to UC as can be observed by the presence of higher amounts of albumin contamination. Western blot analysis also demonstrated the EV surface marker profiles for all 4 precipitation reagents to be different from UC, suggesting that these may have isolated different EV subtypes from UC.

We next tested the 4 PBP protocols using serum from 15 GC patients and 15 healthy controls. We profiled a total of 133 miRNAs and found that the majority (104 miRNAs) could be detected in all EVs isolated using all 4 PBP methods. We identified 11, 5, 5, and 7 EV-miRNA biomarker candidates isolated using lnvt, SBI, Exi, and Han, respectively. The Invt reagent gave the most EV-miRNA candidates and 10 out of these 11 had higher GC detection accuracy (AUC) compared to serum miRNA. We thus showed that EV isolation using PBP can enrich miRNA and improve GC miRNA biomarker performance.

The 11 EV-miRNAs discovered in our pilot study were further validated in an independent set of 20 GC and 20 control serum. Eight out of these 11 EV-miRNAs gave superior improvement in diagnostic signal-to-noise ratio as compared to total circulating miRNAs, validating the improvement in miRNA biomarker performance after EV isolation using PBP. Four of these miRNAs, namely miR-423-5p, miR-484, miR-142-5p, and miR-17-5p, had either been shown to be dysregulated in GC or implicated in GC tumourigenesis/metastasis ⁵⁰⁻⁵⁵.

In summary, we showed that isolation of serum EV-miRNAs improved GC miRNA biomarker performance. In particular, we established the Invt PBP protocol as the one which discovered the highest number of EV-miRNA biomarkers. Using this reagent, we identified 8 EV-miRNA GC biomarkers which were validated in a validation set. Our work has proven the concept that EV-miRNAs indeed can serve as potential diagnostic markers for GC. Further studies are needed to decipher the origin, specific roles and functions of these 8 miRNAs in GC tumourigenesis.

### REFERENCES

1. Esquela-Kerscher A, Slack F J. Oncomirs—microRNAs with a role in cancer [Review Article]. Nature Reviews Cancer. 2006;6: 259. doi: 10.1038/nrc1840.

2. Lim L P, Glasner M E, Yekta S, et al. Vertebrate microRNA genes. Science. 2003;299(5612).

3. Kiss T. Small nucleolar RNAs: an abundant group of noncoding RNAs with diverse cellular functions. Cell. 2002 Apr 19;109(2): 145-8. PubMed PMID: 12007400; eng.

4. Lu M, Zhang Q, Deng M, et al. An Analysis of Human MicroRNA and Disease Associations. PLoS ONE. 2008;3(10): e3420. doi: 10.1371/journal.pone.0003420.

5. Glinge C, Clauss S, Boddum K, et al. Stability of Circulating Blood-Based MicroRNAs—Pre-Analytic Methodological Considerations. PLoS ONE. 2017;12(2): e0167969. doi: 10.1371/journal.pone.0167969.

6. Jung M, Schaefer A, Steiner I, et al. Robust MicroRNA Stability in Degraded RNA Preparations from Human Tissue and Cell Samples [10.1373/clinchem.2009.141580]. Clinical Chemistry. 2010;56(6): 998.

7. Brenner A W, Su G H, Momen-Heravi F. Isolation of Extracellular Vesicles for Cancer Diagnosis and Functional Studies. In: Su GH, editor. Pancreatic Cancer: Methods and Protocols. New York, N.Y.: Springer New York; 2019. p. 229-237.

8. Sanz-Rubio D, Martin-Burriel I, Gil A, et al. Stability of Circulating Exosomal miRNAs in Healthy Subjects. Scientific reports. 2018;8(1): 10306-10306. doi: 10.1038/s41598-018-28748-5. PubMed PMID: 29985466.

9. Zhou X, Zhu W, Li H, et al. Diagnostic value of a plasma microRNA signature in gastric cancer: a microRNA expression analysis. Scientific Reports. 2015;5: 11251. doi: 10.1038/srep11251. PubMed PMID: PMC4462022.

10. Chen K-B, Chen J, Jin X-L, et al. Exosome-mediated peritoneal dissemination in gastric cancer and its clinical applications. Biomedical Reports. 2018;8(6): 503-509. doi: 10.3892/br.2018.1088. PubMed PMID: PMC5954603.

11. Thind A, Wilson C. Exosomal miRNAs as cancer biomarkers and therapeutic targets. Journal of Extracellular Vesicles. 2016;5:10.3402/jev.v5.31292. doi: 10.3402/jev.v5.31292. PubMed PMID: PMC4954869.

12. Wang M, Gu, H., Wang, S., Qian, H., Zhu, W., Zhang, L., Zhao, C., Tao, Y., Xu, W. Circulating miR-17-5p and miR-20a: Molecular markers for gastric cancer. Molecular Medicine Reports. 2012 2012;5(6): 1514-1520.

13. Lin J, Li J, Huang B, et al. Exosomes: Novel Biomarkers for Clinical Diagnosis. The Scientific World Journal. 2015;2015: 657086. doi: 10.1155/2015/657086. PubMed PMID: PMC4322857.

14. Willms E, Cabañas C, Mäger I, et al. Extracellular Vesicle Heterogeneity: Subpopulations, Isolation Techniques, and Diverse Functions in Cancer Progression. Frontiers in Immunology. 2018;9: 738. doi: 10.3389/fimmu.2018.00738. PubMed PMID: PMC5936763.

15. Sohn W, Kim J, Kang S H, et al. Serum exosomal microRNAs as novel biomarkers for hepatocellular carcinoma. Experimental & Molecular Medicine. 2015;47(9):e184. doi: 10.1038/emm.2015.68. PubMed PMID: PMC4650928.

16. Chiam K, Wang T, Watson DI, et al. Circulating Serum Exosomal miRNAs As Potential Biomarkers for Esophageal Adenocarcinoma. Journal of Gastrointestinal Surgery. 2015 2015/07/01;19(7): 1208-1215. doi: 10.1007/s11605-015-2829-9.

17. Li M, Rai A J, Joel DeCastro G, et al. An optimized procedure for exosome isolation and analysis using serum samples: Application to cancer biomarker discovery. Methods. 2015 2015/10/01/;87(Supplement C): 26-30. doi: https://doi.org/10.1016/j.ymeth.2015.03.009.

18. Ogata-Kawata H, lzumiya M, Kurioka D, et al. Circulating Exosomal microRNAs as Biomarkers of Colon Cancer. PLoS ONE. 2014;9(4):e92921. doi: 10.1371/journal.pone.0092921. PubMed PMID: PMC3976275.

19. Taylor D D, Gercel-Taylor C. MicroRNA signatures of tumor-derived exosomes as diagnostic biomarkers of ovarian cancer. Gynecologic Oncology. 2008;110(1): 13-21. doi: 10.1016/j.ygyno.2008.04.033.

20. Marrugo-Ramirez J, Mir M, Samitier J. Blood-Based Cancer Biomarkers in Liquid Biopsy: A Promising Non-Invasive Alternative to Tissue Biopsy. International journal of molecular sciences. 2018;19(10): 2877. doi: 10.3390/ijms19102877. PubMed PMID: 30248975.

21. Momen-Heravi F, Getting S J, Moschos S A. Extracellular vesicles and their nucleic acids for biomarker discovery. Pharmacology & Therapeutics. 2018 2018/08/03/. doi: https://doi.org/10.1016/j.pharmthera.2018.08.002.

22. Furi I, Momen-Heravi F, Szabo G. Extracellular vesicle isolation: present and future. Annals of Translational Medicine. 2017;5(12): 263. doi: 10.2103⁷/ₐtm.2017.03.95. PubMed PMID: PMC5497100.

23. Colombo M, Raposo G, Théry C. Biogenesis, Secretion, and Intercellular Interactions of Exosomes and Other Extracellular Vesicles. Annual Review of Cell and Developmental Biology. 2014 2014/10/11;30(1): 255-289. doi: 10.1146/annurev-cellbio-101512-122326.

24. Li P, Kaslan M, Lee SH, et al. Progress in Exosome Isolation Techniques. Theranostics. 2017;7(3): 789-804. doi: 10.7150/thno.18133. PubMed PMID: 28255367.

25. Lim L P, Glasner M E, Yekta S, et al. Vertebrate MicroRNA Genes [10.1126/science.1080372]. Science. 2003;299(5612): 1540.

26. Chen J, Xu Y, Lu Y, et al. Isolation and Visible Detection of Tumor-Derived Exosomes from Plasma. Analytical Chemistry. 2018 2018/10/29. doi: 10.102¹/ₐcs.analchem.8b03031.

27. Xu H, Liao C, Zuo P, et al. Magnetic-Based Microfluidic Device for On-Chip Isolation and Detection of Tumor-Derived Exosomes. Analytical Chemistry. 2018 2018/09/20. doi: 10.102¹/ₐcs.analchem.8b03272.

28. Tian Y-F, Ning C-F, He F, et al. Highly sensitive detection of exosomes by SERS using gold nanostar@Raman reporter@nanoshell structures modified with a bivalent cholesterol-labeled DNA anchor [10.1039/C8AN01041B]. Analyst. 2018;143(20): 4915-4922. doi: 10.1039/C8AN01041B.

29. An M, Wu J, Zhu J, et al. Comparison of an Optimized Ultracentrifugation Method versus Size-Exclusion Chromatography for Isolation of Exosomes from Human Serum. Journal of Proteome Research. 2018 2018/10/05;17(10): 3599-3605. doi: 10.1021/acs.jproteome.8b00479.

30. Chang M, Chang Y-J, Chao PY, et al. Exosome purification based on PEG-coated Fe3O4 nanoparticles. PloS one. 2018;13(6): e0199438-e0199438. doi: 10.1371/journal.pone.0199438. PubMed PMID: 29933408.

31. Thery C, Zitvogel L, Amigorena S. Exosomes: composition, biogenesis and function [10.1038/nri855]. Nat Rev Immunol. 2002;2(8): 569-579.

32. Yoshikawa M, linuma H, Umemoto Y, et al. Exosome-encapsulated microRNA-223-3p as a minimally invasive biomarker for the early detection of invasive breast cancer. Oncology Letters. 2018;15(6): 9584-9592. doi: 10.3892/01.2018.8457. PubMed PMID: PMC5958689.

33. Xu R, Rai A, Chen M, et al. Extracellular vesicles in cancer—implications for future improvements in cancer care. Nature Reviews Clinical Oncology. 2018 2018/05/23. doi: 10.1038/s41571-018-0036-9.

34. Enderle D, Spiel A, Coticchia CM, et al. Characterization of RNA from Exosomes and Other Extracellular Vesicles Isolated by a Novel Spin Column-Based Method. PLoS ONE. 2015;10(8): e0136133. doi: 10.1371/journal.pone.0136133. PubMed PMID: PMC4552735.

35. Ghosh A, Davey M, Chute I C, et al. Rapid Isolation of Extracellular Vesicles from Cell Culture and Biological Fluids Using a Synthetic Peptide with Specific Affinity for Heat Shock Proteins. PLoS ONE. 2014;9(10): e110443. doi: 10.1371/journal.pone.0110443. PubMed PMID: PMC4201556.

36. Zarovni N, Corrado A, Guazzi P, et al. Integrated isolation and quantitative analysis of exosome shuttled proteins and nucleic acids using immunocapture approaches. Methods. 2015 2015/10/01/;87(Supplement C): 46-58. doi: https://doi.org/10.1016/j.ymeth.2015.05.028.

37. Tauro B J, Greening D W, Mathias R A, et al. Comparison of ultracentrifugation, density gradient separation, and immunoaffinity capture methods for isolating human colon cancer cell line LIM1863-derived exosomes. Methods. 2012 2012/02/01/;56(2): 293-304. doi: https://doi.org/10.1016/j.ymeth.2012.01.002.

38. Malla B, Aebersold D M, Dal Pra A. Protocol for serum exosomal miRNAs analysis in prostate cancer patients treated with radiotherapy. Journal of translational medicine. 2018;16(1): 223-223. doi: 10.1186/s12967-018-1592-6. PubMed PMID: 30103771.

39. Niu Z, Pang R T K, Liu W, et al. Polymer-based precipitation preserves biological activities of extracellular vesicles from an endometrial cell line. PLOS ONE. 2017;12(10): e0186534. doi: 10.1371/journal.pone.0186534.

40. Rider M A, Hurwitz S N, Meckes Jr D G. ExtraPEG: A Polyethylene Glycol-Based Method for Enrichment of Extracellular Vesicles [Article]. Scientific Reports. 2016 04/12/online;6:23978. doi: 10.1038/srep23978 https://www.nature.com/articles/srep23978#supplementary-information.

41. Chen P, Zhao H, Huang J, et al. MicroRNA-17-5p promotes gastric cancer proliferation, migration and invasion by directly targeting early growth response 2. American Journal of Cancer Research. 2016;6(9): 2010-2020. PubMed PMID: PMC5043110.

42. Zhang X, Yan Z, Zhang J, et al. Combination of hsa-miR-375 and hsa-miR-142-5p as a predictor for recurrence risk in gastric cancer patients following surgical resection. Annals of Oncology. 2011;22(10): 2257-2266. doi: 10.1093/annonc/mdq758.

43. Liu R, Zhang C, Hu Z, et al. A five-microRNA signature identified from genome-wide serum microRNA expression profiling serves as a fingerprint for gastric cancer diagnosis. European Journal of Cancer. 2011;47(5): 784-791. doi: 10.1016/j.ejca.2010.10.025.

44. Liu J, Wang X, Yang X, et al. miRNA423-5p regulates cell proliferation and invasion by targeting trefoil factor 1 in gastric cancer cells. Cancer Letters. 2014;347(1): 98-104. doi: 10.1016/j.canlet.2014.01.024.

45. Bu H, He D, He X, et al. Exosomes: isolation, analysis, and applications in cancer detection and therapy. ChemBioChem. 2018;0(ja). doi: 10.1002/cbic.201800470.

46. Kalishwaralal K, Kwon W Y, Park K S. Exosomes for non-invasive cancer monitoring. Biotechnology Journal. 2018;0(ja): 1800430. doi: 10.1002/biot.201800430.

47. Kiss T. Small Nucleolar RNAs. Cell. 2002;109(2): 145-148. doi: 10.1016/s0092-8674(02)00718-3.

48. Zarovni N, Corrado A, Guazzi P, et al. Integrated isolation and quantitative analysis of exosome shuttled proteins and nucleic acids using immunocapture approaches. Methods. 2015 Oct. 1;87: 46-58. doi: 10.1016/j.ymeth.2015.05.028. PubMed PMID: 26044649; eng.

49. Kim C H, Kim H K, Rettig R L, et al. miRNA signature associated with outcome of gastric cancer patients following chemotherapy. BMC Medical Genomics. 2011;4:79-79. doi: 10.1186/1755-8794-4-79. PubMed PMID: PMC3287139.

### REFERENCES (FOR EXAMPLES 18 TO 21)

1. Hayes J, Peruzzi P P, Lawler S: MicroRNAs in cancer: biomarkers, functions and therapy. Trends in Molecular Medicine 2014, 20: 460-469.

2. Wahid F, Shehzad A, Khan T, Kim Y Y: MicroRNAs: Synthesis, mechanism, function, and recent clinical trials. Biochimica et Biophysica Acta (BBA)—Molecular Cell Research 2010, 1803: 1231-1243.

3. Lu M, Zhang Q, Deng M, Miao J, Guo Y, Gao W, Cui Q: An Analysis of Human MicroRNA and Disease Associations. PLoS ONE 2008, 3: e3420.

4. Esquela-Kerscher A, Slack F J: Oncomirs—microRNAs with a role in cancer. Nature Reviews Cancer 2006, 6: 259.

5. Kiss T: Small Nucleolar RNAs. Cell 2002, 109: 145-148.

6. Lim L P, Glasner M E, Yekta S, Burge C B, Bartel D P: Vertebrate MicroRNA Genes. Science 2003, 299: 1540.

7. Jung M, Schaefer A, Steiner I, Kempkensteffen C, Stephan C, Erbersdobler A, Jung K: Robust MicroRNA Stability in Degraded RNA Preparations from Human Tissue and Cell Samples. Clinical Chemistry 2010, 56: 998.

8. Glinge C, Clauss S, Boddum K, Jabbari R, Jabbari J, Risgaard B, Tomsits P, Hildebrand B, Kaab S, Wakili R, Jespersen T, Tfelt-Hansen J: Stability of Circulating Blood-Based MicroRNAs—Pre-Analytic Methodological Considerations. PLoS ONE 2017, 12: e0167969.

9. Sanz-Rubio D, Martin-Burriel I, Gil A, Cubero P, Forner M, Khalyfa A, Marin J M: Stability of Circulating Exosomal miRNAs in Healthy Subjects. Scientific reports 2018, 8:10306-10306.

10. Uratani R, Toiyama Y, Kitajima T, Kawamura M, Hiro J, Kobayashi M, Tanaka K, Inoue Y, Mohri Y, Mori T, Kato T, Goel A, Kusunoki M: Diagnostic Potential of Cell-Free and Exosomal MicroRNAs in the Identification of Patients with High-Risk Colorectal Adenomas. PLoS ONE 2016, 11: e0160722.

11. Zhou X, Zhu W, Li H, Wen W, Cheng W, Wang F, Wu Y, Qi L, Fan Y, Chen Y, Ding Y, Xu J, Qian J, Huang Z, Wang T, Zhu D, Shu Y, Liu P: Diagnostic value of a plasma microRNA signature in gastric cancer: a microRNA expression analysis. Scientific Reports 2015, 5: 11251.

12. Chen K-B, Chen J, Jin X-L, Huang Y, Su Q-M, Chen L: Exosome-mediated peritoneal dissemination in gastric cancer and its clinical applications. Biomedical Reports 2018, 8: 503-509.

13. Thind A, Wilson C: Exosomal miRNAs as cancer biomarkers and therapeutic targets. Journal of Extracellular Vesicles 2016, 5:10.3402/jev.v3405.31292.

14. Thery C, Zitvogel L, Amigorena S: Exosomes: composition, biogenesis and function. Nat Rev Immunol 2002, 2: 569-579.

15. Lin J, Li J, Huang B, Liu J, Chen X, Chen X-M, Xu Y-M, Huang L-F, Wang X-Z: Exosomes: Novel Biomarkers for Clinical Diagnosis. The Scientific World Journal 2015, 2015: 657086.

16. Xu R, Rai A, Chen M, Suwakulsiri W, Greening D W, Simpson R J: Extracellular vesicles in cancer—implications for future improvements in cancer care. Nature Reviews Clinical Oncology 2018.

17. Wu H-H, Lin W-c, Tsai K-W: Advances in molecular biomarkers for gastric cancer: miRNAs as emerging novel cancer markers. Expert reviews in molecular medicine, 16: e1-e1.

18. Marrugo-Ramirez J, Mir M, Samitier J: Blood-Based Cancer Biomarkers in Liquid Biopsy: A Promising Non-Invasive Alternative to Tissue Biopsy. International journal of molecular sciences 2018, 19: 2877.

19. Ogata-Kawata H, lzumiya M, Kurioka D, Honma Y, Yamada Y, Furuta K, Gunji T, Ohta H, Okamoto H, Sonoda H, Watanabe M, Nakagama H, Yokota J, Kohno T, Tsuchiya N: Circulating Exosomal microRNAs as Biomarkers of Colon Cancer. PLoS ONE 2014, 9: e92921.

20. Chiam K, Wang T, Watson D I, Mayne G C, Irvine T S, Bright T, Smith L, White I A, Bowen J M, Keefe D, Thompson S K, Jones M E, Hussey D J: Circulating Serum Exosomal miRNAs As Potential Biomarkers for Esophageal Adenocarcinoma. Journal of Gastrointestinal Surgery 2015, 19: 1208-1215.

21. Momen-Heravi F, Getting S J, Moschos S A: Extracellular vesicles and their nucleic acids for biomarker discovery. Pharmacology & Therapeutics 2018.

22. Taylor D D, Gercel-Taylor C: MicroRNA signatures of tumor-derived exosomes as diagnostic biomarkers of ovarian cancer. Gynecologic Oncology 2008, 110: 13-21.

23. Li M, Rai A J, Joel DeCastro G, Zeringer E, Barta T, Magdaleno S, Setterquist R, Vlassov A V: An optimized procedure for exosome isolation and analysis using serum samples: Application to cancer biomarker discovery. Methods 2015, 87: 26-30.

24. Sohn W, Kim J, Kang S H, Yang S R, Cho J-Y, Cho H C, Shim S G, Paik Y-H: Serum exosomal microRNAs as novel biomarkers for hepatocellular carcinoma. Experimental & Molecular Medicine 2015, 47: e184.

25. Brenner A W, Su G H, Momen-Heravi F: Isolation of Extracellular Vesicles for Cancer Diagnosis and Functional Studies. Pancreatic Cancer: Methods and Protocols. Edited by Su GH. New York, N.Y.: Springer New York, 2019. pp. 229-237.

26. Colombo M, Raposo G, Thery C: Biogenesis, Secretion, and Intercellular Interactions of Exosomes and Other Extracellular Vesicles. Annual Review of Cell and Developmental Biology 2014, 30: 255-289.

27. Willms E, Cabanas C, Mager I, Wood M J A, Vader P: Extracellular Vesicle Heterogeneity: Subpopulations, Isolation Techniques, and Diverse Functions in Cancer Progression. Frontiers in Immunology 2018, 9: 738.

28. Furi I, Momen-Heravi F, Szabo G: Extracellular vesicle isolation: present and future. Annals of translational medicine 2017, 5: 263-263.

29. Li P, Kaslan M, Lee SH, Yao J, Gao Z: Progress in Exosome Isolation Techniques. Theranostics 2017, 7: 789-804.

30. An M, Wu J, Zhu J, Lubman D M: Comparison of an Optimized Ultracentrifugation Method versus Size-Exclusion Chromatography for Isolation of Exosomes from Human Serum. Journal of Proteome Research 2018, 17: 3599-3605.

31. Tian Y-F, Ning C-F, He F, Yin B-C, Ye B-C: Highly sensitive detection of exosomes by SERS using gold nanostar@Raman reporter@nanoshell structures modified with a bivalent cholesterol-labeled DNA anchor. Analyst 2018, 143: 4915-4922.

32. Chen J, Xu Y, Lu Y, Xing W: Isolation and Visible Detection of Tumor-Derived Exosomes from Plasma. Analytical Chemistry 2018.

33. Xu H, Liao C, Zuo P, Liu Z, Ye B-C: Magnetic-Based Microfluidic Device for On-Chip Isolation and Detection of Tumor-Derived Exosomes. Analytical Chemistry 2018.

34. Chang M, Chang Y-J, Chao P Y, Yu Q: Exosome purification based on PEG-coated Fe3O4 nanoparticles. PloS one 2018, 13: e0199438-e0199438.

35. Baranyai T, Herczeg K, Onodi Z, Voszka I, Modos K, Marton N, Nagy G, Mäger I, Wood M J, El Andaloussi S, Pálinkás Z, Kumar V, Nagy P, Kittel Á, Buzás El, Ferdinandy P, Giricz Z: Isolation of Exosomes from Blood Plasma: Qualitative and Quantitative Comparison of Ultracentrifugation and Size Exclusion Chromatography Methods. PLoS ONE 2015, 10: e0145686.

36. Greening D W, Xu R, Ji H, Tauro B J, Simpson R J: A Protocol for Exosome Isolation and Characterization: Evaluation of Ultracentrifugation, Density-Gradient Separation, and Immunoaffinity Capture Methods. Proteomic Profiling: Methods and Protocols. Edited by Posch A. New York, N.Y.: Springer New York, 2015. pp. 179-209.

37. Wong L L, Zou R, Zhou L, Lim J Y, Phua D C Y, Liu C, Chong J P C, Ng J Y X, Liew O W, Chan S P, Chen Y-T, Chan M M Y, Yeo P S D, Ng T P, Ling L H, Sim D, Leong K T G, Ong H Y, Jaufeerally F, Wong R, Chai P, Low A F, Lund M, Devlin G, Troughton R, Cameron V A, Doughty R N, Lam C S P, Too H P, Richards A M: Combining Circulating MicroRNA and NT-proBNP to Detect and Categorize Heart Failure Subtypes. Journal of the American College of Cardiology 2019, 73: 1300-1313.

38. Enderle D, Spiel A, Coticchia C M, Berghoff E, Mueller R, Schlumpberger M, Sprenger-Haussels M, Shaffer J M, Lader E, Skog J, Noerholm M: Characterization of RNA from Exosomes and Other Extracellular Vesicles Isolated by a Novel Spin Column-Based Method. PLoS ONE 2015, 10: e0136133.

39. Ghosh A, Davey M, Chute I C, Griffiths S G, Lewis S, Chacko S, Barnett D, Crapoulet N, Fournier S, Joy A, Caissie M C, Ferguson A D, Daigle M, Meli M V, Lewis S M, Ouellette R J: Rapid Isolation of Extracellular Vesicles from Cell Culture and Biological Fluids Using a Synthetic Peptide with Specific Affinity for Heat Shock Proteins. PLoS ONE 2014, 9: e110443.

40. Zarovni N, Corrado A, Guazzi P, Zocco D, Lari E, Radano G, Muhhina J, Fondelli C, Gavrilova J, Chiesi A: Integrated isolation and quantitative analysis of exosome shuttled proteins and nucleic acids using immunocapture approaches. Methods 2015, 87: 46-58.

41. Sharma P, Ludwig S, Muller L, Hong C S, Kirkwood J M, Ferrone S, Whiteside T L: Immunoaffinity-based isolation of melanoma cell-derived exosomes from plasma of patients with melanoma. Journal of extracellular vesicles 2018, 7: 1435138-1435138.

42. Tauro B J, Greening D W, Mathias R A, Ji H, Mathivanan S, Scott A M, Simpson R J: Comparison of ultracentrifugation, density gradient separation, and immunoaffinity capture methods for isolating human colon cancer cell line LIM1863-derived exosomes. Methods 2012, 56: 293-304.

43. Rider M A, Hurwitz S N, Meckes Jr D G: ExtraPEG: A Polyethylene Glycol-Based Method for Enrichment of Extracellular Vesicles. Scientific Reports 2016, 6: 23978.

44. Niu Z, Pang R T K, Liu W, Li Q, Cheng R, Yeung W S B: Polymer-based precipitation preserves biological activities of extracellular vesicles from an endometrial cell line. PLOS ONE 2017, 12: e0186534.

45. Malla B, Aebersold D M, Dal Pra A: Protocol for serum exosomal miRNAs analysis in prostate cancer patients treated with radiotherapy. Journal of translational medicine 2018, 16: 223-223.

46. Kalishwaralal K, Kwon W Y, Park K S: Exosomes for non-invasive cancer monitoring. Biotechnology Journal 2018, 0: 1800430.

47. Bu H, He D, He X, Wang K: Exosomes: isolation, analysis, and applications in cancer detection and therapy. ChemBioChem 2018, 0.

48. Nedaeinia R, Manian M, Jazayeri M H, Ranjbar M, Salehi R, Sharifi M, Mohaghegh F, Goli M, Jahednia S H, Avan A, Ghayour-Mobarhan M: Circulating exosomes and exosomal microRNAs as biomarkers in gastrointestinal cancer. Cancer Gene Therapy 2016, 24: 48.

49. Xie J X, Fan X, Drummond C A, Majumder R, Xie Y, Chen T, Liu L, Haller S T, Brewster P S, Dworkin L D, Cooper C J, Tian J: MicroRNA profiling in kidney disease: Plasma versus plasma-derived exosomes. Gene 2017, 627: 1-8.

50. Wang M, Gu, H., Wang, S., Qian, H., Zhu, W., Zhang, L., Zhao, C., Tao, Y., Xu, W: Circulating miR-17-5p and miR-20a: Molecular markers for gastric cancer. Molecular Medicine Reports 2012, 5: 1514-1520.

51. Zhang X, Yan Z, Zhang J, Gong L, Li W, Cui J, Liu Y, Gao Z, Li J, Shen L, Lu Y: Combination of hsa-miR-375 and hsa-miR-142-5p as a predictor for recurrence risk in gastric cancer patients following surgical resection. Annals of Oncology 2011, 22: 2257-2266.

52. Liu R, Zhang C, Hu Z, Li G, Wang C, Yang C, Huang D, Chen X, Zhang H, Zhuang R, Deng T, Liu H, Yin J, Wang S, Zen K, Ba Y, Zhang C-Y: A five-microRNA signature identified from genome-wide serum microRNA expression profiling serves as a fingerprint for gastric cancer diagnosis. European Journal of Cancer 2011, 47: 784-791.

53. Liu J, Wang X, Yang X, Liu Y, Shi Y, Ren J, Guleng B: miRNA423-5p regulates cell proliferation and invasion by targeting trefoil factor 1 in gastric cancer cells. Cancer Letters 2014, 347: 98-104.

54. Kim C H, Kim H K, Rettig R L, Kim J, Lee E T, Aprelikova 0, Choi I J, Munroe D J, Green J E: miRNA signature associated with outcome of gastric cancer patients following chemotherapy. BMC Medical Genomics 2011, 4: 79-79.

55. Chen P, Zhao H, Huang J, Yan X, Zhang Y, Gao Y: MicroRNA-17-5p promotes gastric cancer proliferation, migration and invasion by directly targeting early growth response 2. American Journal of Cancer Research 2016, 6: 2010-2020.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. A method of diagnosing a gastric cancer, in which the method comprises detecting, in a sample from or of an individual:
the expression level of an miRNA selected from the group consisting of: hsa-miR-484, hsa-miR-186-5p, hsa-miR-142-5p, hsa-miR-320d, hsa-miR-320a, hsa-miR-320b and hsa-miR-17-5p;
in which an altered expression level of the miRNA as compared to the expression level of the miRNA in a sample from or of an individual known not to be suffering from gastric cancer indicates that the individual is suffering, or is likely to be suffering, from gastric cancer.

2. A method according to claim 1, in which the miRNA is detected in an extracellular vesicle (EV) and:
(a) hsa-miR-484 comprises a polynucleotide sequence having miRBase Accession Number MIMAT0002174 or a variant, homologue, derivative or fragment thereof such as a sequence having 75%, 80%, 85%, 90%, 95%, 96%, 97%, 98% or 99% sequence identity thereto and comprising hsa-miR-484 activity and in which the altered expression level comprises an increased expression level of hsa-miR-484;
(b) hsa-miR-186-5p comprises a polynucleotide sequence having miRbase Accession Number MIMAT0000456 or a variant, homologue, derivative or fragment thereof such as a sequence having 75%, 80%, 85%, 90%, 95%, 96%, 97%, 98% or 99% sequence identity thereto and comprising hsa-miR-186-5p activity and in which the altered expression level comprises an increased expression level of hsa-miR-186-5p;
(c) hsa-miR-142-5p comprises a polynucleotide sequence having miRBase Accession Number MIMAT0000433 or a variant, homologue, derivative or fragment thereof such as a sequence having 75%, 80%, 85%, 90%, 95%, 96%, 97%, 98% or 99% sequence identity thereto and comprising hsa-miR-142-5p activity and in which the altered expression level comprises a decreased expression level of hsa-miR-142-5p;
(d) hsa-miR-320d comprises a polynucleotide sequence having miRBase Accession Number MIMAT0006764 or a variant, homologue, derivative or fragment thereof such as a sequence having 75%, 80%, 85%, 90%, 95%, 96%, 97%, 98% or 99% sequence identity thereto and comprising hsa-miR-320d activity and in which the altered expression level comprises an increased expression level of hsa-miR-320d;
(e) hsa-miR-320a comprises a polynucleotide sequence having miRBase Accession Number MI0000542 or a variant, homologue, derivative or fragment thereof such as a sequence having 75%, 80%, 85%, 90%, 95%, 96%, 97%, 98% or 99% sequence identity thereto and comprising hsa-miR-320a activity and in which the altered expression level comprises an increased expression level of hsa-miR-320a;
(f) hsa-miR-320b comprises a polynucleotide sequence having miRBase Accession Number MIMAT0005792 or a variant, homologue, derivative or fragment thereof such as a sequence having 75%, 80%, 85%, 90%, 95%, 96%, 97%, 98% or 99% sequence identity thereto and comprising hsa-miR-320b activity and in which the altered expression level comprises an increased expression level of hsa-miR-320b; or
(g) hsa-miR-17-5p comprises a polynucleotide sequence having miRBase Accession Number MIMAT0000070 or a variant, homologue, derivative or fragment thereof such as a sequence having 75%, 80%, 85%, 90%, 95%, 96%, 97%, 98% or 99% sequence identity thereto and comprising hsa-miR-17-5p activity and in which the altered expression level comprises a decreased expression level of hsa-miR-17-5p.

3. A method according to claim 1 or 2, in which the method comprises detecting the expression level in a sample, such as in an extracellular vesicle (EV) in or of the sample, of two or more such miRNAs, for example, three miRNAs, four miRNAs, five miRNAs, six miRNAs or seven miRNAs in the group.

4. A method according to claim 1, 2 or 3, in which the method further comprises detecting the expression level in a sample, such as in an extracellular vesicle (EV) in or of the sample, of hsa-miR-423-5p (miRBase Accession Number MIMAT0004748) or a variant, homologue, derivative or fragment thereof such as a sequence having at least 75%, 80%, 85%, 90%, 95%, 96%, 97%, 98% or 99% sequence identity thereto and comprising hsa-miR-423-5p activity.

5. A method according to any preceding claim, in which:
(a) an expression level of hsa-miR-484 of 0.39 or more;
(b) an expression level of hsa-miR-186-5p of 0.15 or more;
(c) an expression level of hsa-miR-142-5p of −0.35 or less;
(d) an expression level of hsa-miR-320d of 0.48 or more;
(e) an expression level of hsa-miR-320a of 0.49 or more;
(f) an expression level of hsa-miR-320b of 0.4 or more;
(g) an expression level of hsa-miR-17-5p of −0.37 or less;
(h) an expression level of hsa-miR-423-5p of 0.54 or more;
each measured as log₂(expression level of individual/expression level of control), in which control designates the expression level of that miRNA in a sample, such as in an extracellular vesicle (EV) in or of the sample, from or of an individual known not to be suffering from gastric cancer, is indicative of gastric cancer.

6. A method according to any preceding claim, in which the sample comprises a bodily fluid sample such as a nasopharyngeal secretion, urine, serum, lymph, saliva, anal and vaginal secretions, perspiration or semen.

7. A method according to any preceding claim, in which extracellular vesicles (EV) are isolated from the sample using polymer based precipitation.

8. A method according to any preceding claim, in which the detection comprises polymerase chain reaction, such as real-time polymerase chain reaction (RT-PCR), multiplex polymerase chain reaction (multiplex PCR), Northern Blot, RNAse protection, microarray hybridisation or RNA sequencing.

9. A combination of two or more nucleic acids specified in any preceding claim or probes capable of binding specifically thereto, such as a combination of nucleic acids immobilised on a substrate, preferably in the form of a microarray or as a multiplex polymerase chain reaction (PCR) kit.

10. A combination according to claim 9, comprising probes capable of binding specifically thereto to each of hsa-miR-484 (miRBase Accession Number MIMAT0002174), hsa-miR-186-5p (miRbase Accession Number MIMAT0000456), hsa-miR-142-5p (miRBase Accession Number MIMAT0000433), hsa-miR-320d (miRBase Accession Number MIMAT0006764), hsa-miR-320a (miRBase Accession Number MI0000542), hsa-miR-320b (miRBase Accession Number MIMAT0005792), hsa-miR-17-5p (miRBase Accession Number MIMAT0000070) and hsa-miR-423-5p (miRBase Accession Number MIMAT0004748).

11. An miRNA selected from the group consisting of hsa-miR-484, hsa-miR-186-5p, hsa-miR-142-5p, hsa-miR-320d, hsa-miR-320a, hsa-miR-320b and hsa-miR-17-5p or a variant, homologue, derivative or fragment thereof such as a sequence having at least 75%, 80%, 85%, 90%, 95%, 96%, 97%, 98% or 99% sequence identity thereto for use in a method of detecting or determining the severity of gastric cancer.

12. A pharmaceutical composition comprising two or more miRNAs selected from the group consisting of: hsa-miR-484, hsa-miR-186-5p, hsa-miR-142-5p, hsa-miR-320d, hsa-miR-320a, hsa-miR-320b, hsa-miR-17-5p and hsa-miR-423-5p, or a variant, homologue, derivative or fragment thereof such as a sequence having at least 75%, 80%, 85%, 90%, 95%, 96%, 97%, 98% or 99% sequence identity thereto together with a pharmaceutically acceptable excipient, carrier or diluent.

13. A diagnostic kit for gastric cancer, the kit comprising sequences capable of binding to two or more miRNAs selected from the group consisting of: hsa-miR-484, hsa-miR-186-5p, hsa-miR-142-5p, hsa-miR-320d, hsa-miR-320a, hsa-miR-320b, hsa-miR-17-5p and hsa-miR-423-5p, or a variant, homologue, derivative or fragment thereof such as a sequence having at least 75%, 80%, 85%, 90%, 95%, 96%, 97%, 98% or 99% sequence identity thereto together with instructions for use.

14. A method of treatment of a gastric cancer in an individual, the method comprising performing a method according to any of claims 1 to 8 and, where the individual is determined to be suffering from, or likely to suffer from, gastric cancer, administering to the individual a treatment for gastric cancer.

15. A method of treating gastric cancer in an individual, the method comprising:
(a) receiving results of an assay that measures the expression level of an miRNA selected from the group consisting of: hsa-miR-484, hsa-miR-186-5p, hsa-miR-142-5p, hsa-miR-320d, hsa-miR-320a, hsa-miR-320b and hsa-miR-17-5p (optionally together with hsa-miR-423-5p) or a variant, homologue, derivative or fragment thereof such as a sequence having at least 75%, 80%, 85%, 90%, 95%, 96%, 97%, 98% or 99% sequence identity thereto in a sample of or from an individual, in which the results show the expression level of the miRNA in the sample;
(b) if the expression of the miRNA in the sample is modulated compared to a reference expression level of an miRNA, the reference expression level being the expression level of the miRNA in a sample of or from an individual known not to be suffering from gastric cancer, thereby providing or predicting an indication of gastric cancer in the individual, administering a treatment for gastric cancer;
in which the expression level of the miRNA is optionally detected in an extracellular vesicle (EV) of or from the sample.

16. A method for treating gastric cancer in an individual, comprising:
(a) obtaining the results of an analysis of the expression level of an miRNA selected from the group consisting of: hsa-miR-484, hsa-miR-186-5p, hsa-miR-142-5p, hsa-miR-320d, hsa-miR-320a, hsa-miR-320b and hsa-miR-17-5p (optionally together with hsa-miR-423-5p) or a variant, homologue, derivative or fragment thereof such as a sequence having at least 75%, 80%, 85%, 90%, 95%, 96%, 97%, 98% or 99% sequence identity thereto in a sample of or from an individual; and
(b) administering a treatment for gastric cancer to the individual if the expression level of the miRNA is modulated compared to a reference expression level, the reference expression level being the expression level of the miRNA in a sample of or from an individual known not to be suffering from gastric cancer;
in which the expression level of the miRNA is optionally detected in an extracellular vesicle (EV) of or from the sample.
